(19) United States
(12) Patent Application Publication
(10) Pub. No.: US 2023/0382952 A1
(43) Pub. Date: Nov. 30, 2023

(54) COMPOSITIONS COMPRISING HIV ENVELOPES TO INDUCE HIV-1 ANTIBODIES

(71) Applicants: Bette T. KORBER, Los Alamos, NM (US); Kshitij G. WAGH, Los Alamos, NM (US); Duke University, Durham, NC (US)

(72) Inventors: Kevin O. SAUNDERS, Durham, NC (US); Barton F. HAYNES, Durham, NC (US); Bette T. KORBER, Los Alamos, NM (US); Kshitij G. WAGH, Los Alamos, NM (US)

(21) Appl. No.: 18/032,497

(22) PCT Filed: Oct. 19, 2021
(86) PCT No.: PCT/US2021/055699

§ 371 Date: Apr. 18, 2023

Related U.S. Application Data
(60) Provisional application No. 63/093,675, filed on Oct. 19, 2020.

(51) Int. Cl. C07K 14/005; A61K 39/21

(52) U.S. Cl. CPC C07K 14/005; A61K 39/21; C12N 2740/16122; C12N 2740/16134; A61K 2039/55555

(57) ABSTRACT

The invention is directed to modified HIV-1 envelopes, compositions comprising these modified envelopes, nucleic acids encoding these modified envelopes, compositions comprising these nucleic acids, and methods of using these modified HIV-1 envelopes and/or these nucleic acids to induce immune responses.

This application claims the benefit of and priority to U.S. Application Ser. No. 63/093,675 filed Oct. 19, 2020 the content of which is hereby incorporated by reference in its entirety.

This invention was made with government support under Center for HIV/AIDS Vaccine Immunology-Immunogen Design grant UM1-AI144371 from the NIH, NIAID, Division of AIDS. The government has certain rights in the invention. The United States government also has certain rights in this invention pursuant to Contract No. 89233218CNA000001 between the United States Department of Energy and Triad National Security, LLC for the operation of Los Alamos National Laboratory.

### SEQUENCE LISTING

The instant application contains a Sequence Listing which has been submitted electronically in ASCII format and is hereby incorporated by reference in its entirety. Said ASCII copy, created on Oct. 19, 2021, is named 1234300_00378WO1_SL.txt and is 776,647 bytes in size. The Sequence Listing ASCII copy includes SEQ ID NOs: 1-185 which form part of the description filed in the form of an Annex C/ST.25 text file. SEQ ID NOs: 186-196 also form part of the description and are included herewith in the description and figures.

### TECHNICAL FIELD

The present invention relates in general, to a composition suitable for use in inducing anti-HIV-1 antibodies, and, in particular, to immunogenic compositions comprising envelope proteins and nucleic acids to induce cross-reactive neutralizing antibodies and increase their breadth of coverage. The invention also relates to methods of inducing such broadly neutralizing anti-HIV-1 antibodies using such compositions.

### BACKGROUND

The development of a safe and effective HIV-1 vaccine is one of the highest priorities of the scientific community working on the HIV-1 epidemic. While anti-retroviral treatment (ART) has dramatically prolonged the lives of HIV-1 infected patients, ART is not routinely available in developing countries.

### SUMMARY OF THE INVENTION

In certain embodiments, the invention provides compositions and methods for induction of an immune response, for example cross-reactive (broadly) neutralizing (bn) Ab induction.

In certain aspects the invention provides a selection of a series of immunogens and immunogen designs for induction of neutralizing HIV-1 antibodies, e.g. but not limited to V3 glycan epitope targeting antibodies, the selection comprising envelopes as follows: 1) CH848.d0949.10.17 DT (also referred to as CH848.d0949.10.17.N133D.N138T), 2) CH848.d0949.10.17 (also referred to as CH848.d0949.10.17WT), 3) CH848.d0808.15.15, 4) CH848.d0358.80.06, 5) CH848.d1432.5.41, 6) CH848.d1621.4.44 and 7) CH848.d1305.10.35. See Example 1 and Tables 3, 4, 5, 6, 7, 8 and 9. In some embodiments the selection comprises additional HIV-1 Envs, P0402.c2.11 and ZM246F.

In certain embodiments, CH848.d0949.10.17DT envelope comprises additional modifications D230N.H289N.P291S.E169K and is referred to as CH848.d0949.10.17 DTe. In certain embodiments, CH848.d0949.10.17 envelope comprises additional modifications D230N.H289N.P291S.E169K and is referred to as CH848.d0949.10.17WTe. In non-limiting embodiments, the envelope in the selections for immunization are included as trimers, protein and/or mRNA. In non-limiting embodiments, the envelope in the selections for immunization are included as nanoparticles, protein and/or mRNA. The designation scNP refers to a non-limiting embodiment of a protein nanoparticle formed by sortase conjugation reaction. In non-limiting embodiments, nanoparticles comprise fusion proteins, for example ferritin-envelope fusion proteins.

In certain aspects the invention provides a recombinant protein or nucleic acid encoding a recombinant protein as described in Example 1 and Tables 3-9. In certain aspects the invention provides a selection of HIV-1 envelopes for use as prime and boost immunogens in methods to induce HIV-1 neutralizing antibodies.

In certain aspects the invention provides compositions comprising a selection of HIV-1 envelopes and/or nucleic acids encoding these envelopes as described herein for example but not limited to designs as described herein. Without limitations, these selected combinations comprise envelopes which provide representation of the sequence (genetic) and antigenic diversity of the HIV-1 envelope variants which lead to the induction of V1V2 glycan and V3 glycan antibody lineages.

In certain aspects the invention provides a recombinant HIV-1 envelope comprising a 17 amino acid (17aa) V1 region, lacking glycosylation at position N133 and N138 (HXB2 numbering), comprising glycosylation at N301 (HXB2 numbering) and N332 (HXB2 numbering), comprising modifications wherein glycan holes are filled (D230N_H289N_P291S (HXB2 numbering)), comprising the “GDIR” (SEQ ID NO:1) or “GDIK” (SEQ ID NO:2), or any trimer stabilization modifications, UCA targeting modification, immunogenicity modification, or combinations thereof, for example but not limited to those described in Table 2. In certain embodiments the recombinant envelope optionally comprises any combinations of these modifications.

In certain embodiments, the envelope is not a CH848 10.17 DT variant described previously in PCT Publication No. WO2018161049.

In certain embodiments the envelope is a protomer which could be comprised in a stable trimer.

In certain embodiments the envelope comprises additional mutations stabilizing the envelope trimer. In certain embodiments these including but are not limited to SOSIP mutations. In certain embodiments mutations are selected from sets F1-F14, VT1-VT8 mutations described herein, or any combination or subcombination within a set. In certain embodiments, the selected mutations are F14. In other embodiments, the selected mutations are VT8. In certain embodiments, the selected mutations are F14 and VT8 combined.

In certain embodiments, the invention provides a recombinant HIV-1 envelope of FIG. 1, FIG. 2, FIG. 3, or FIG. 20. In certain embodiments, the invention provides a nucleic acid encoding any of the recombinant envelopes. In certain embodiments, the nucleic acids comprise an mRNA formulated for use as a pharmaceutical composition.

In certain embodiments the inventive designs comprise specific changes (D230N_H289N_P291S (HXB2 numbering)) which fill glycan holes with the introduction of new glycosylation sites to prevent the binding of strain-specific antibodies that could hinder broad neutralizing antibody development. See Wagh, Kshitij et al. “Completeness of HIV-1 Envelope Glycan Shield at Transmission Determines Neutralization Breadth.” Cell reports vol. 25, 4 (2018): 893-908.e7. doi:10.1016/j.celrep.2018.09.087; Crooks, Ema T et al. “Vaccine-Elicited Tier 2 HIV-1 Neutralizing Antibodies Bind to Quaternary Epitopes Involving Glycan-Deficient Patches Proximal to the CD4 Binding Site.” PLoS pathogens vol. 11, 5 e1004932. 29 May 2015, doi:10.1371/journal.ppat.1004932.

In certain embodiments, the inventive designs comprise modifications, including without limitation fusion of the HIV-1 envelope with ferritin using linkers between the HIV-1 envelope and ferritin designed to optimize ferritin nanoparticle assembly.

In certain embodiments, the invention provides HIV-1 envelopes comprising Lys327 (HXB2 numbering) optimized for administration as a prime to initiate V3 glycan antibody lineage, e.g. DH270 antibody lineage.

In certain embodiments, the invention provides HIV-1 envelopes comprising Lys169 (HXB2 numbering).

In certain embodiments, the invention provides a composition comprising any one of the inventive envelopes or nucleic acid sequences encoding the same. In certain embodiments, the nucleic acid is mRNA. In certain embodiments, the mRNA is comprised in a lipid nano-particle (LNP).

In certain embodiments, the invention provides compositions comprising a nanoparticle which comprises any one of the envelopes of the invention.

In certain embodiments, the invention provides compositions comprising a nanoparticle which comprises any one of the envelopes of the invention, wherein the nanoparticle is a ferritin self-assembling nanoparticle.

In certain embodiments, the invention provides a method of inducing an immune response in a subject comprising administering an immunogenic composition comprising any one of the stabilized recombinant HIV-1 envelopes of the invention. In certain embodiments, the composition is administered as a prime and/or a boost. In certain embodiments, the composition comprises nanoparticles. In certain embodiments, methods of the invention further comprise administering an adjuvant.

In certain embodiments, the invention provides a composition comprising a plurality of nanoparticles comprising a plurality of the recombinant HIV-1 envelopes/trimers of the invention. In non-limiting embodiments, the envelopes/trimers of the invention are multimeric when comprised in a nanoparticle. The nanoparticle size is suitable for delivery. In non-liming embodiments the nanoparticles are ferritin based nanoparticles.

In certain aspects, the invention provides nucleic acids comprising sequences encoding proteins of the invention. In certain embodiments, the nucleic acids are DNAs. In certain embodiments, the nucleic acids are mRNAs. In certain aspects, the invention provides expression vectors comprising the nucleic acids of the invention.

In certain aspects, the invention provides a pharmaceutical composition comprising mRNAs encoding proteins of the invention. In certain embodiments, these are optionally formulated in lipid nanoparticles (LNPs). In certain embodiments, the mRNAs are modified. Modifications include without limitations modified ribonucleotides, poly-A tail, 5′cap.

In certain aspects the invention provides nucleic acids encoding the inventive protein designs. In non-limiting embodiments, the nucleic acids are mRNA, modified or unmodified, suitable for use any use, e.g but not limited to use as pharmaceutical compositions. In certain embodiments, the nucleic acids are formulated in lipid, such as but not limited to LNPs. Non-limiting embodiments include LNPs without polyethylene glycol.

In certain aspects the invention provides a recombinant HIV-1 envelope selected from the envelopes listed in Table 3 or Table 4, FIGS. 1-3, or FIG. 20. In certain aspects the invention provides a selection of envelopes from Table 3-9, FIGS. 1-3, or FIG. 20, or Example 1 for use as immunogens in methods to induce antibody responses to HIV-1 envelope.

In certain embodiments, the envelope is a protomer comprised in a trimer. In some embodiments, the envelope is comprised in a stable trimer. In certain embodiments, the nanoparticle comprises any one of the envelopes Table 3-9, FIGS. 1-3, FIG. 20, or Example 1, for example without limitation for use as immunogens. In certain embodiments, the nanoparticle is ferritin self-assembling nanoparticle.

In certain aspects, the invention provides a composition comprising a nanoparticle and a carrier, wherein the nanoparticle comprises any one of the trimers of envelopes Table 3-9, FIGS. 1-3, FIG. 20, or Example 1. In certain embodiments, the nanoparticle is a ferritin self-assembling nanoparticle. In certain embodiments, the nanoparticle comprises multimers of trimers. Provided also are method for using these compositions comprising nanoparticles.

In certain aspects, the invention provides a method of inducing an immune response in a subject comprising administering an immunogenic composition comprising any one of the recombinant envelopes of Table 3-9, FIGS. 1-3, FIG. 20, or Example 1, or compositions comprising these envelopes and/or nanoparticles, in an amount sufficient to induce an immune response. In certain embodiments, the composition is administered as a prime.

In certain embodiments, the composition is administered as a boost.

In certain aspects, the invention provides a nucleic acid encoding any of the recombinant envelopes and methods for their use to induce immune response in a subject in need thereof.

In certain aspects the invention provides a method of inducing an immune response comprising administering an immunogenic composition comprising a prime immunogen from Table 4 followed by at least one boost immunogen from Table 4, wherein in some embodiments the boost immunogens are administered in the order appearing in Table 4, or Table 5-9 in an amount sufficient to induce an immune response. In certain embodiments, the prime is one of the CH848.0949.10.17DT or CH848.0949.10.17DTe designs, for example in Table 3 or Table 4. In certain embodiments, the first boost is one of the CH848.0949.10.17WT or CH848.0949.10.17WTe designs, for example in Table 3 or Table 4.

In certain embodiments, the methods further comprise administering a boost from Table 4, or Table 5-9, wherein the boost is CH848.0808.15.15 in any suitable form.

In certain embodiments, the methods further comprise administering a boost from Table 4, wherein the boost is CH848.0358.80.06 in any suitable form.

In certain embodiments, the methods further comprise administering a boost from Table 4, wherein the boost is CH848.1432.5.41 in any suitable form.

In certain embodiments, the methods further comprise administering a boost from Table 4, wherein the boost is CH848.1621.4.44 in any suitable form.

In certain embodiments, the methods further comprise administering a boost from Table 4, wherein the boost is CH848.1305.10.35 in any suitable form.

In certain embodiments, the methods further comprise comprising administering a boost from Table 4, wherein the boost is P0402.c2.11 (G) in any suitable form.

In certain embodiments, the methods further comprise administering a boost from Table 4, wherein the boost is ZM246F (C) in any suitable form.

In certain embodiments, the prime and/or boost immunogen are administered as a nanoparticle. In certain embodiments, the nanoparticle is a ferritin nanoparticle. In certain embodiments, the methods further comprise administering the prime and/or boost immunogen as a mRNA-LNP formulation.

In certain embodiments, the methods further comprise administering any suitable adjuvant.

### BRIEF DESCRIPTION OF THE DRAWINGS

The patent or application file contains at least one drawing executed in color. To conform to the requirements for PCT patent applications, many of the figures presented herein are black and white representations of images originally created in color.

FIGS. 1A-1H show non-limiting examples of envelopes designs and sequences described in Table 3. FIGS. 1A-1D show non-limiting embodiments of nucleic acid sequence. Nucleotide sequences have the signal peptide sequences. FIGS. 1E-1H show amino acid sequences. Amino acid sequences lack the signal peptide sequences. FIG. 1I shows correlation of envelope names with HV numbers and SEQ ID Nos.

FIGS. 2A part (i)-2B part (xxxi) shows non-limiting examples of envelope designs and sequences described in Table 4—envelopes CH848.0808.15.15, CH848.1621.4.44, CH848.1305.10.35, P0402.c2.11 (G), ZM246F (C). This figure shows selected sequences from Table 4. FIG. 2A parts (i)-(xxxi) show non-limiting embodiments of nucleic acid sequence and FIG. 2B parts (i)-(xxxi) show amino acid sequences. In the amino acid sequences the signal sequence is underlined. FIG. 2C shows correlation of envelope names with HV numbers and SEQ ID Nos.

FIGS. 3A-3T show non-limiting examples of designs and sequences based on envelopes CH848.0358.80.06 and CH848.1432.5.41. FIGS. 3A-3J show non-limiting embodiments of nucleic acid sequence and FIGS. 3K-T show amino acid sequences. In the amino acid sequences the signal sequence is underlined. FIG. 3U shows correlation of envelope names with HV numbers and SEQ ID Nos.

FIGS. 4A-B show signature sites. Signatures are amino acids/glycans that are statistically enriched in one group of viruses vs other. FIG. 4A shows systematic definition of bnAb education sites. To systematically probe overlapping signature sites consider the following: Overlapping signature sites; Borderline: Signature defined in one dataset, if also a borderline signature in the other; Phylogenetic or not; p<0.05; Same association with the same feature. For IA2 breadth gain, only one borderline signature found in autologous dataset (NxST230)-retained this. These analyses gave six sites—these are amino acid positions HXB2 numbering: 230, 241, 300, 301, 325, 328, where 241, 300 & 325 are phylogenetic. FIG. 4B shows bNAb Education Signature Sites.

FIG. 5 shows logo plots of bnAb education sites. IA4-sensitive: CH848.d0949.10.17 matches all sensitive variants. IA2 breadth gain: 230 NxST associated with IA2 breadth gain (only autologous). E-325 is quite rare in heterologous viruses. IA1 breadth gain: Breadth gain variants: K-241, G/Y-300 & K-328. N-325 recognized at very low frequency. DH270.6/.4 gain: Y-300 & K-328 are better recognized. N-325 is still rarely recognized. Resistance to all DH270 lineage: Main routes of escape loss of NxST301 & N325 (heterologous only NxST-332 viruses considered). Panel on the left is Global Panel and panel on the right is Autologous Panel—on the X-axis are positions of bnAb education sites 230, 241, 300, 301k 325, and 328. See Example 1. Amino acid colors as follows: Blue (represented by darkest gray in grayscale image) shows initial lineage sensitivity; green (represented by medium gray in grayscale image) shows breadth gain; red (represented by medium gray plus asterisk in grayscale image) shows resistance signature; lightest grey and black show non-significant amino acids. In this figure adjacent amino acids do not represent a continuous peptide, but rather amino acids at the signature sites positions: 230, 241, 300, 301, 325 and 328.

FIG. 6 shows an embodiment of an immunogen design comprising prime and boost. In this figure adjacent amino acids do not represent a continuous peptide, but rather amino acids at the signature sites positions: 230, 241, 300, 301, 325 and 328.

FIG. 7 shows one embodiment of a selection of immunogens. Non-limiting embodiments of these envelopes are listed in Tables 3 and 4. In this figure adjacent amino acids do not represent a continuous peptide, but rather amino acids at the signature sites positions: 230, 241, 300, 301, 325 and 328.

FIG. 8 shows a selection of immunogens for breadth gain beyond DH270.6. Some N-325 viruses are sensitive to DH270.6. Chose this for a gentler heterologous boost. The most sensitive virus P0402.c2.11 (subtype G, tier 2) was the only virus that also provided coverage at other sites (27 & 85). No virus lacking NxST 301 was neutralized by DH270.6. From CATNAP we found ZM246F that is sensitive all other V3g bNAbs, but not tested on DH270.6. In this figure adjacent amino acids do not represent a continuous peptide, but rather amino acids at the signature sites positions: 230, 241, 300, 301, 325 and 328.

FIGS. 9A-B show vaccine elicitation of serum neutralization in mouse studies Mu563-1 and Mu563-2. FIG. 9A shows DH270 UCA3 VH^{+/-}, VL^{+/-} mouse immunization regimen—mice were immunized 4× with prime and 4× with boost 1. FIG. 9B shows serum neutralization titer as serum dilution required to inhibit 50% of virus replication one week after the final immunization. Horizontal line represents group geometric mean. Each symbol represents an individual mouse.

FIGS. 10A-B show vaccine elicitation of serum neutralization in mouse studies Mu565-1 and Mu565-2. FIG. 10A shows DH270 UCA3 VH^{+/-}, VL^{+/-} mouse immunization regimen—mice were immunized 4× with prime and 2× with boost 1 and 2× with boost 2. FIG. 10B shows serum neutralization titer as serum dilution required to inhibit 50% of virus replication one week after the final immunization. Horizontal line represents group geometric mean. Each symbol represents an individual mouse. The Mu563 and Mu565 studies compared the boosting effects of HV1301335 and HV1302164 preceded by the HV1302145 envelope or administered directly after HV1301925 prime. The regimen including HV1301335 was superior to the regimen with HV1302164 in eliciting heterologous neutralizing. The heterologous neutralization of 92RW020 was enhanced by including the HV1302145 envelope as the second boost (compare Mu563-1 vs Mu565-1).

FIGS. 11A-11C show vaccine elicitation of serum neutralization in mouse studies Mu486-2 and Mu486-1. FIG. 11A shows DH270 UCA3 VH^{+/-}, VL^{+/-} mouse immunization regimen. FIGS. 11B-C show serum neutralization titer against autologous (FIG. 11B) and heterologous (FIG. 11C) viruses shown as serum dilution required to inhibit 50% of virus replication one week after the final immunization. N133D N138T matches the priming immunogen for both groups. N332T is a knockout mutation for the antibody of interest DH270. SVA is a negative control unrelated virus. Horizontal line represents group geometric mean. Each symbol represents an individual mouse. This study compared different boosting immunogens. Boosting with CH848.D949.10.17 (Mu486-2) elicited superior serum neutralization titers compared to boosting with a sequence of 5 different envelopes (Mu486-1). This study also investigated vaccine induction of critical mutations for antibody function. Amino acid sequence comparison between vaccine-induced antibodies and DH270 natural lineage antibodies. These antibodies were induced by vaccination and show that vaccination is inducing somatic mutation of DH270 antibodies. Mutations in the VH that are critical to select for are G57R and R98T. Both of these somatic mutations are selected by the vaccine regimen in group 2. In the VL of DH270, S27Y and L48Y are critical for neutralization activity. Both S27Y and L48Y were selected for in the vaccine-elicited antibodies. Prime-boost vaccination is eliciting antibodies with the critical somatic mutations needed for broad neutralization.

FIGS. 12A-12B show vaccine elicitation of serum neutralization in mouse study Mu534-1. FIG. 12A shows DH270 UCA3 VH^{+/-}, VL^{+/-} mouse immunization regimen. FIG. 12B shows serum neutralization titer against autologous and heterologous viruses shown as serum dilution required to inhibit 50% of virus replication one week after the final immunization. N133D N138T matches the priming immunogen for both groups. N332T is a knockout mutation for the antibody of interest DH270. SVA is a negative control unrelated virus. Horizontal line represents group geometric mean. Each symbol represents an individual mouse. This study is a repeat of Mu486-2. This study showed that CH848.10.17 DT scNP followed by 10.17 trimer induction of heterologous neutralizing antibodies was reproducible. Induction of 92RW020 neutralization is a virus that is sensitive to affinity matured DH270 antibodies but not the UCA. Serum antibodies from the vaccinate mice neutralize 92RW020 indicating the DH270 antibodies have evolved.

FIG. 13 shows neutralization breadth elicited by prime-boost vaccine regimen. DH270 UCA3 VH^{+/-}, VL^{+/-} mouse immunization regimen as in FIG. 11—mouse studies Mu486-2 and Mu486-1. FIG. 13B. MAb neutralization titer IC50 as mcg per mL required to inhibit 50% of virus replication. Ab730526 was isolated from group 1. The remaining antibodies were isolated from group 2. NT, not tested. These antibodies were induced by vaccination. These vaccine-induced antibodies shown here exhibit broad neutralization. Their neutralization is superior to Dh270.I5.6 which is the first inferred node of the DH270.6 phylogeny. Each antibody has a second version with an additional mutation added (_X), which shows that the artificial addition of DH270 somatic mutations to the antibody further improves neutralization breadth. These artificially-mutated antibodies indicate the mutations that are needed to be selected by the next set of vaccine immunogens.

FIGS. 14A-D show next generation sequencing of heavy chain variable regions shows vaccine selection of critical functional improbable mutations needed for DH270 antibody affinity maturation. FIG. 14A shows each DH270 UCA3 VH^{+/-}, VL^{+/-} mouse immunization regimen. FIGS. 14B-D show the frequency of the observed somatic mutation. Adjuvant only groups are shown as Adj only GLA-SE for protein immunization and Adj only LNP for mRNA. Group median is shown by horizontal bars. Mu546 and Mu547 studies delivered the envelopes as mRNAs. All other groups used proteins.

FIGS. 15A-C show next generation sequencing of light chain variable regions shows vaccine selection of critical functional improbable mutations needed for DH270 antibody affinity maturation. FIG. 15A shows each DH270 UCA3 VH^{+/-}, VL^{+/-} mouse immunization regimen. FIGS. 15B-C show the frequency of the observed somatic mutation. Adjuvant only groups are shown as Adj only GLA-SE for protein immunization and Adj only LNP for mRNA. Group median is shown by horizontal bars. Mu546 and Mu547 studies delivered the envelopes as mRNAs. All other groups used proteins. Mouse studies 563 and 565 included the D358.80.06 DS.ch.SOSIP and D526.25.05 DS.ch.SOSIP as boosting immunogens. Based on the frequency of somatic mutations observed in DH270 sequences, D358.80.06 DS.ch.SOSIP and D526.25.05 DS.ch.SOSIP did not select for higher frequencies of VH (G57R, R98T) mutations or VL (L48Y, S27Y) mutations than immunizing with CH848.D949.10.17N133D/N138T alone (Mu408 or Mu445 group 5). mRNA immunization selected for higher frequencies of VL (L48Y, S27Y) mutations than protein immunization with the same envelope.

FIG. 16 shows DH270 phylogenetic tree.

FIG. 17 shows reactivity of the sequential Env signature-based vaccine envelopes with DH270 lineage members. Biolayer interferometry binding magnitude determined for DH270 antibodies through the affinity maturation process. Binding has been normalized to loading response. Envelopes were selected based on neutralization sensitivity to different members of the DH270 lineage. Recombinant envelopes were generated based on the viruses selected by neutralization signature analysis. The envelope reactivity with antibodies at different stages of maturation in the DH270 lineage was assessed and the envelopes show a staged pattern of reactivity. The staged reactivity showed that certain DH270 antibodies acquired reactivity with selected envelopes at specific points of affinity maturation. For example, P0402 envelope did not bind to lowly mutated antibodies (Dh270 UCA, I5.6, or I3.6), but the antibody lineage acquired binding once it mutated to the DH270.1 stage of maturation. This differential binding is expected to facilitate selection of antibodies during the affinity maturation process. Administering these envelopes in sequence could select for affinity maturation of DH270-like antibodies into a bnAb.

FIG. 18 shows one embodiment of a design for the production of trimeric HIV-1 Env on ferritin nanoparticles. In some embodiments the Sortase A tag is LPSTGG (SEQ ID NO: 25) which is modified from LPSTG (SEQ ID NO: 26) because an additional Gly residue helps accelerate the reaction rate.

FIGS. 19A-19G show correlation of envelopes with SEQ ID Nos.

FIGS. 20A-K shows non-limiting embodiments of HIV-1 envelopes used in animal studies in Example 2. FIG. 20A-20E shows non-limiting embodiments of nucleic acids. FIG. 20F-20J shows non-limiting embodiments of amino acids sequences with a signal peptide. FIG. 20K shows correlation of envelope names with HV numbers and SEQ ID NOs.

### DETAILED DESCRIPTION OF THE INVENTION

The development of a safe, highly efficacious prophylactic HIV-1 vaccine is of paramount importance for the control and prevention of HIV-1 infection. A major goal of HIV-1 vaccine development is the induction of broadly neutralizing antibodies (bnAbs) (Immunol. Rev. 254: 225-244, 2013). BnAbs are protective in rhesus macaques against SHIV challenge, but as yet, are not induced by current vaccines.

The invention provides methods of using these pan bnAb envelope immunogens.

In certain aspect, the invention provides compositions for immunizations to induce lineages of broad neutralizing antibodies. In certain embodiments, there is some variance in the immunization regimen; in some embodiments, the selection of HIV-1 envelopes may be grouped in various combinations of primes and boosts, either as nucleic acids, proteins, or combinations thereof. In certain embodiments the compositions are pharmaceutical compositions which are immunogenic. In certain embodiments, the compositions comprise amounts of envelopes which are therapeutic and/or immunogenic.

In one aspect the invention provides a composition for a prime boost immunization regimen comprising any one of the envelopes described herein, or any combination thereof wherein the envelope is a prime or boost immunogen. In certain embodiments the composition for a prime boost immunization regimen comprises one or more envelopes described herein.

In certain embodiments, the compositions contemplate nucleic acid, as DNA and/or RNA, or recombinant protein immunogens either alone or in any combination. In certain embodiments, the methods contemplate genetic, as DNA and/or RNA, immunization either alone or in combination with recombinant envelope protein(s).

In some embodiments the antigens are nucleic acids, including but not limited to mRNAs which could be modified and/or unmodified. See US Pub 20180028645A1, US Pub 20170369532, US Pub 20090286852, US Pub 20130111615, US Pub 20130197068, US Pub 20130261172, US Pub 20150038558, US Pub 20160032316, US Pub 20170043037, US Pub 20170327842, the content of each of which is incorporated by reference in its entirety. mRNAs delivered in LNP formulations have advantages over non-LNPs formulations. See US Pub 20180028645A1.

In certain embodiments the nucleic acid encoding an envelope is operably linked to a promoter inserted an expression vector. In certain aspects the compositions comprise a suitable carrier. In certain aspects the compositions comprise a suitable adjuvant.

In certain embodiments the induced immune response includes induction of antibodies, including but not limited to autologous and/or cross-reactive (broadly) neutralizing antibodies against HIV-1 envelope. Various assays that analyze whether an immunogenic composition induces an immune response, and the type of antibodies induced are known in the art and are also described herein.

In certain aspects the invention provides an expression vector comprising any of the nucleic acid sequences of the invention, wherein the nucleic acid is operably linked to a promoter. In certain aspects the invention provides an expression vector comprising a nucleic acid sequence encoding any of the polypeptides of the invention, wherein the nucleic acid is operably linked to a promoter. In certain embodiments, the nucleic acids are codon optimized for expression in a mammalian cell, in vivo or in vitro. In certain aspects the invention provides nucleic acids comprising any one of the nucleic acid sequences of invention. In certain aspects the invention provides nucleic acids consisting essentially of any one of the nucleic acid sequences of invention. In certain aspects the invention provides nucleic acids consisting of any one of the nucleic acid sequences of invention. In certain embodiments the nucleic acid of the invention, is operably linked to a promoter and is inserted in an expression vector. In certain aspects the invention provides an immunogenic composition comprising the expression vector.

In certain aspects the invention provides a composition comprising at least one of the nucleic acid sequences of the invention. In certain aspects the invention provides a composition comprising any one of the nucleic acid sequences of invention. In certain aspects the invention provides a composition comprising at least one nucleic acid sequence encoding any one of the polypeptides of the invention.

The envelope used in the compositions and methods of the invention can be a gp160, gp150, gp145, gp140, gp120, gp41, N-terminal deletion variants as described herein, cleavage resistant variants as described herein, or codon optimized sequences thereof. In certain embodiments the composition comprises envelopes as trimers. In certain embodiments, envelope proteins are multimerized, for example trimers are attached to a particle such that multiple copies of the trimer are attached and the multimerized envelope is prepared and formulated for immunization in a human. In certain embodiments, the compositions comprise envelopes, including but not limited to trimers as a particulate, high-density array on liposomes or other particles, for example but not limited to nanoparticles. In some embodiments, the trimers are in a well ordered, near native like or closed conformation. In some embodiments the trimer compositions comprise a homogenous mix of native like trimers. In some embodiments the trimer compositions comprise at least 85%, 90%, 95% native like trimers.

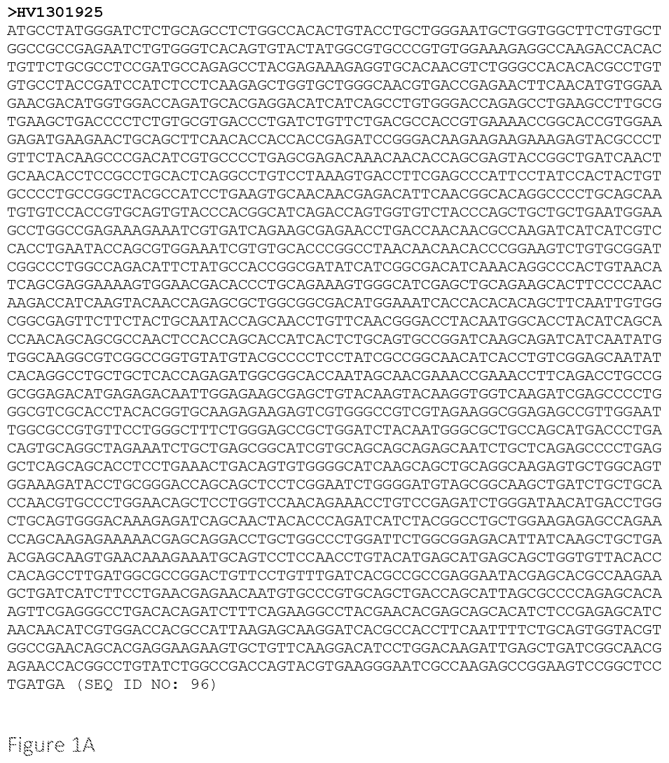

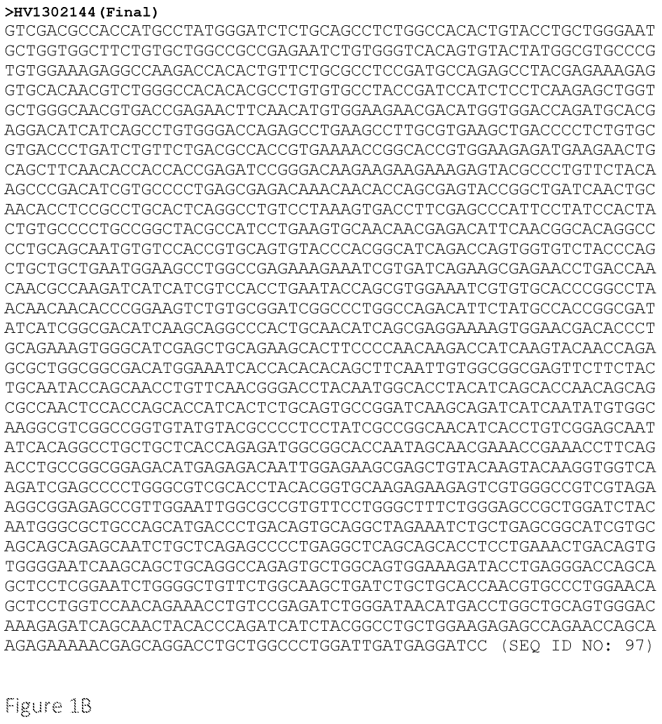

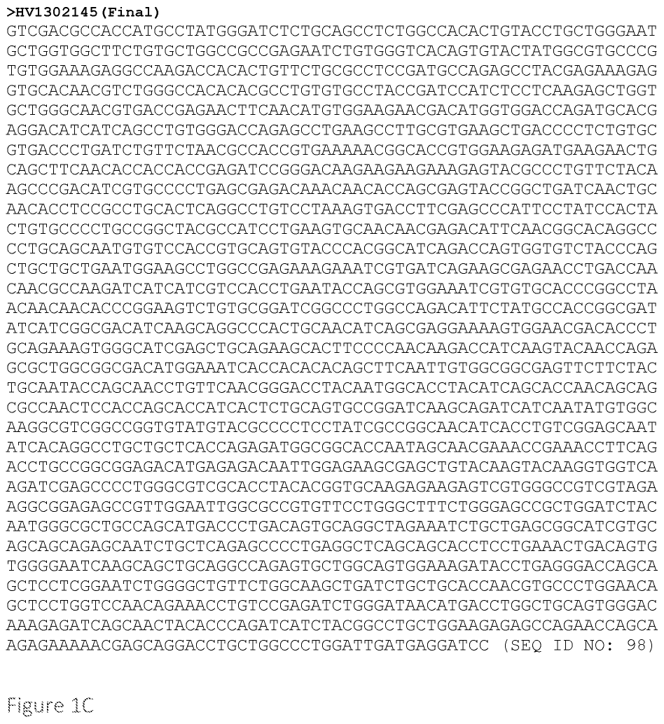

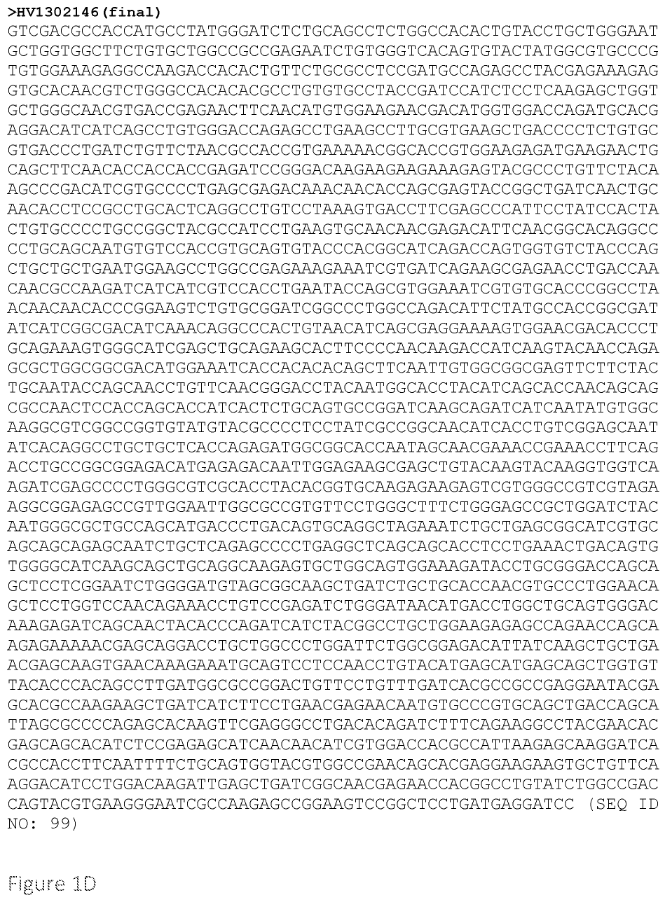

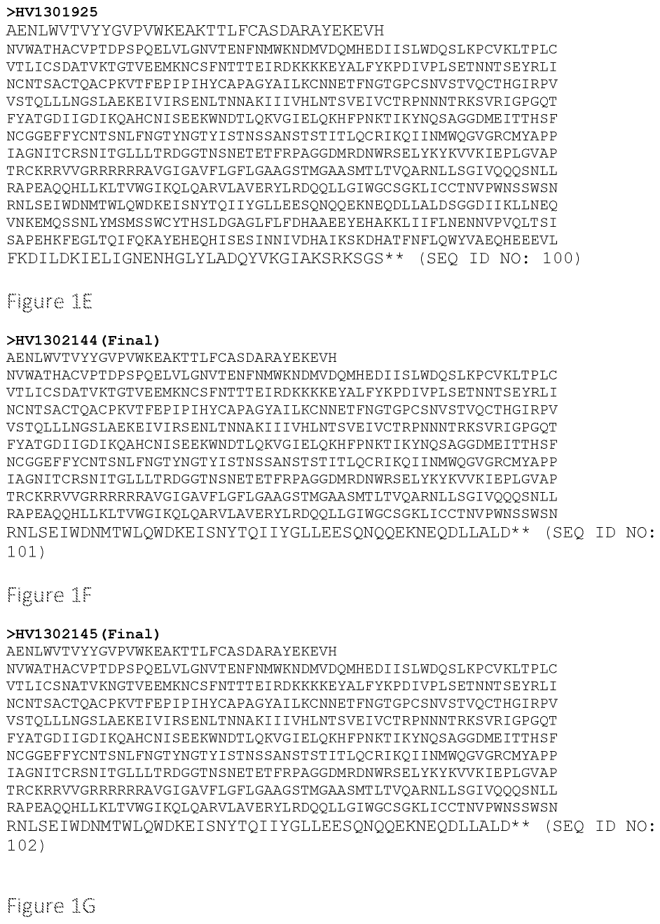

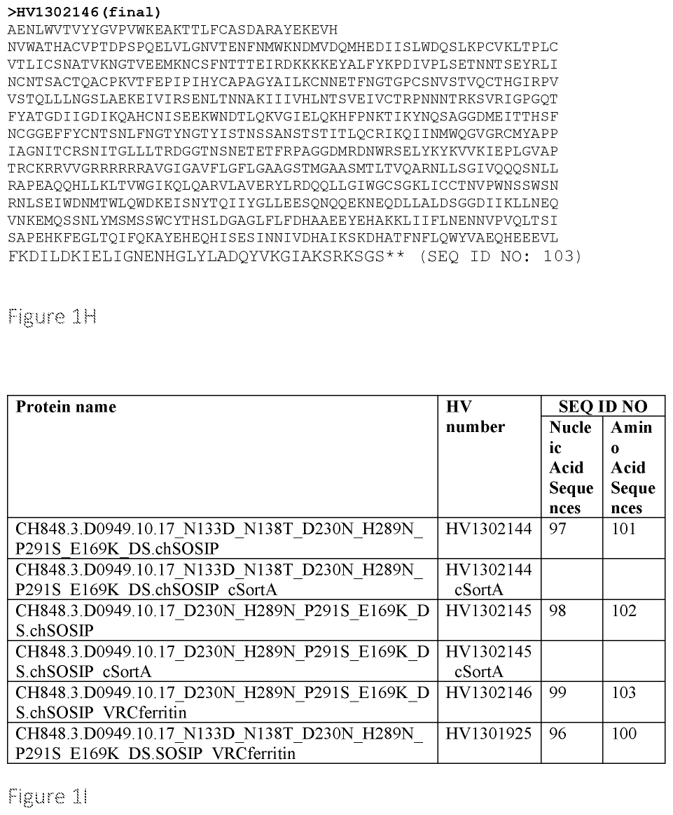

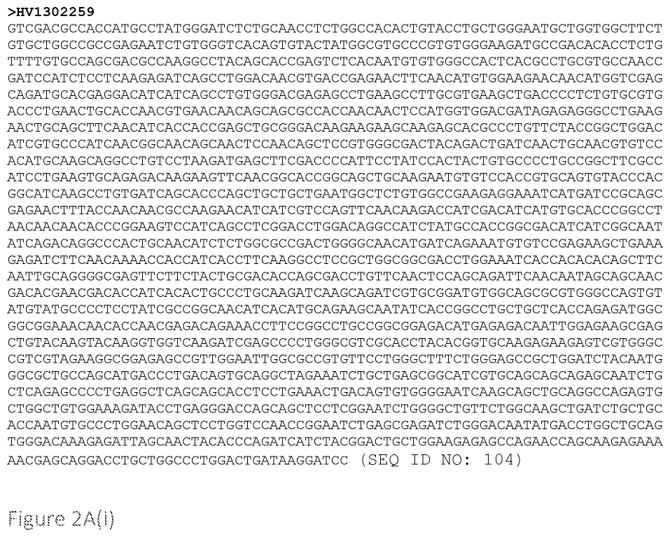

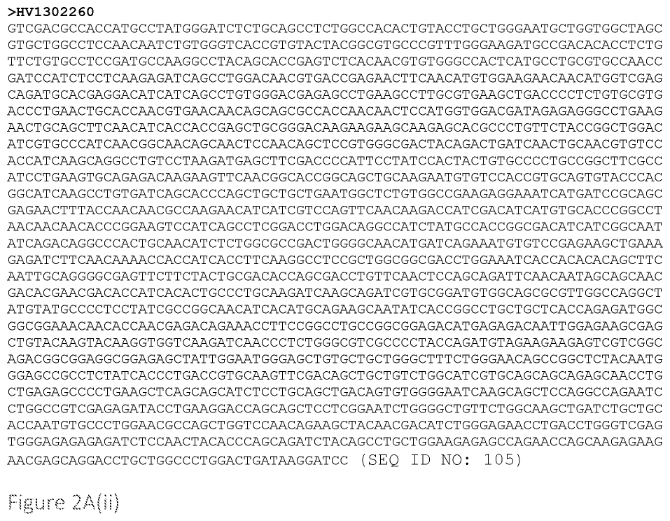

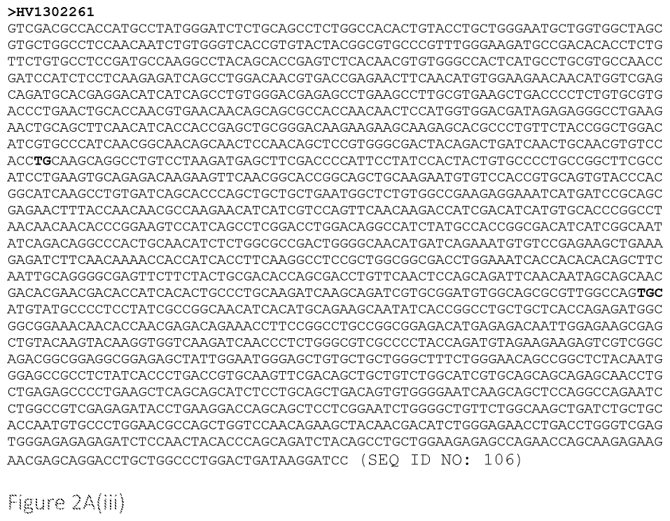

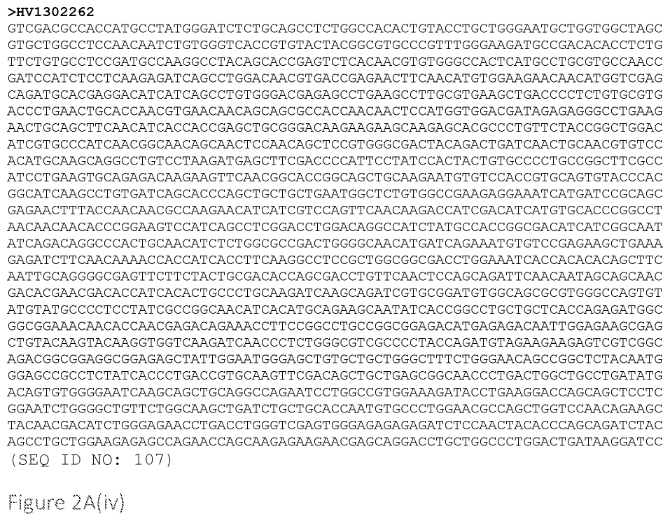

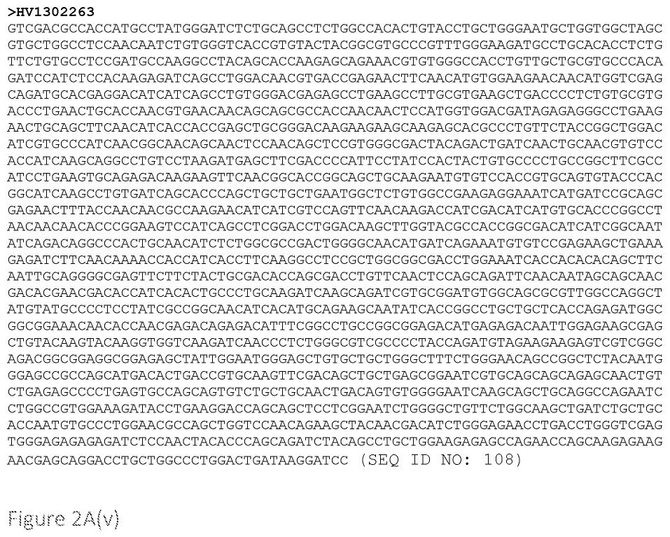

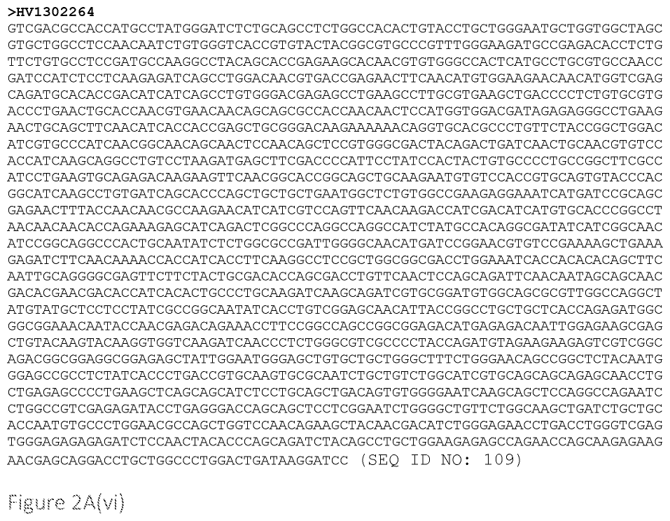

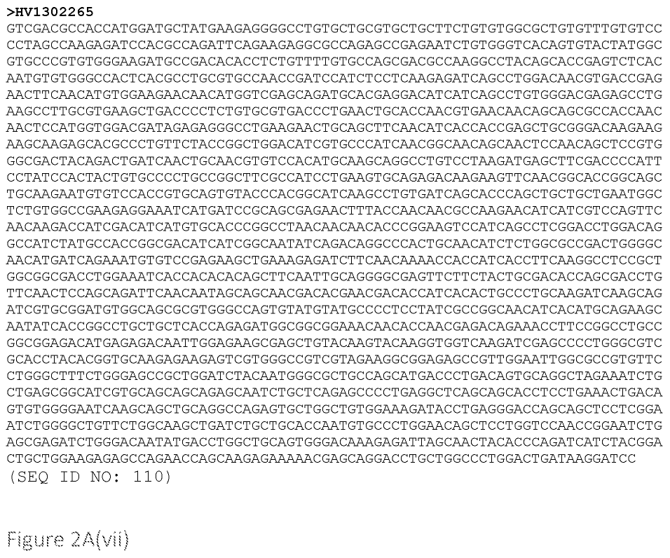

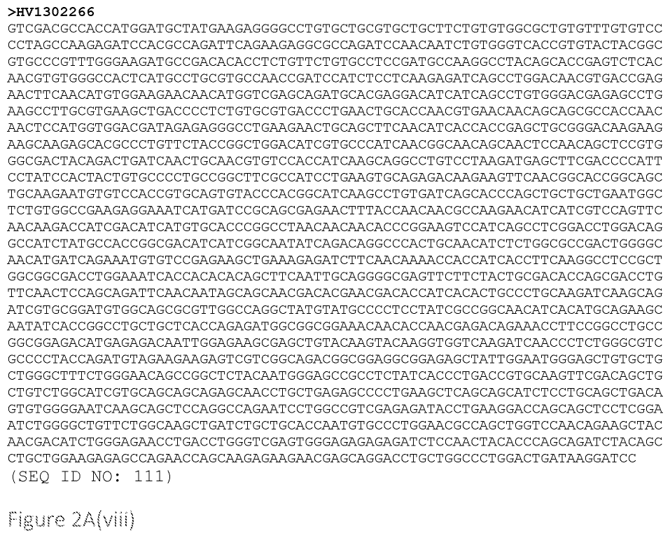

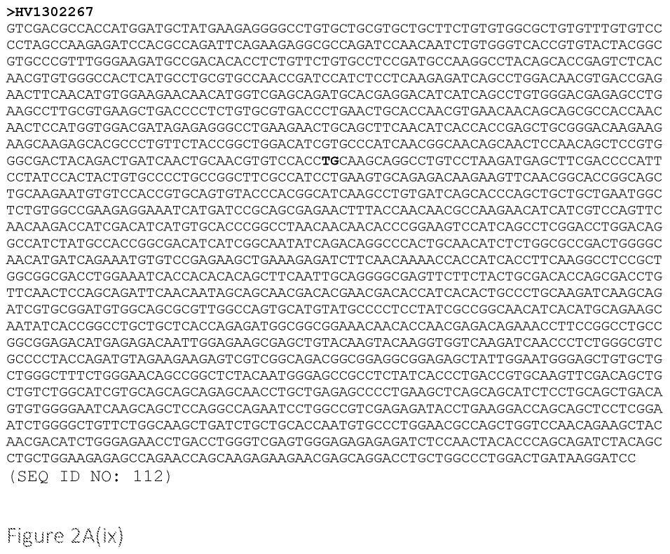

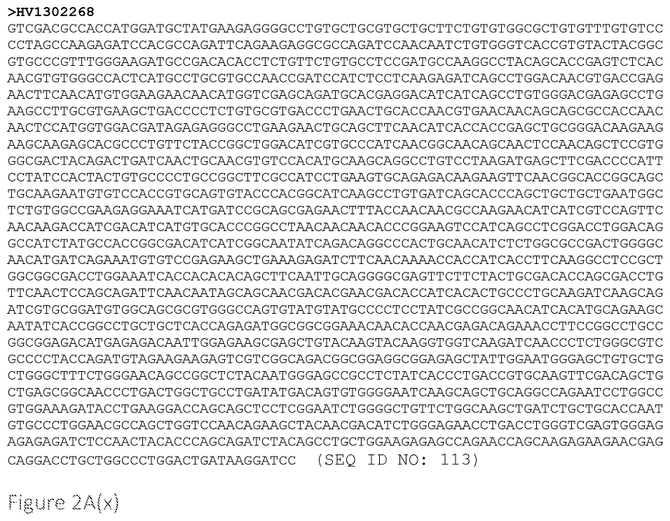

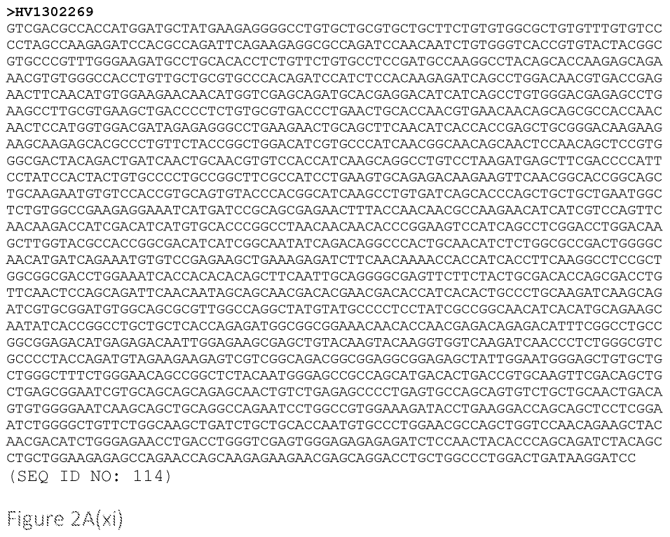

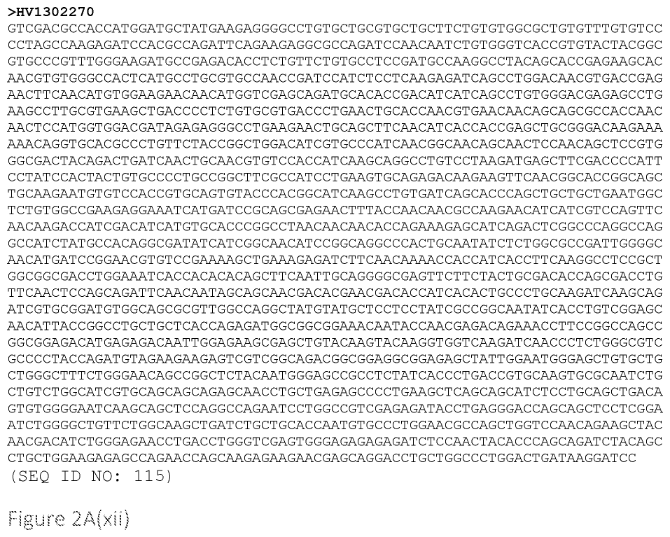

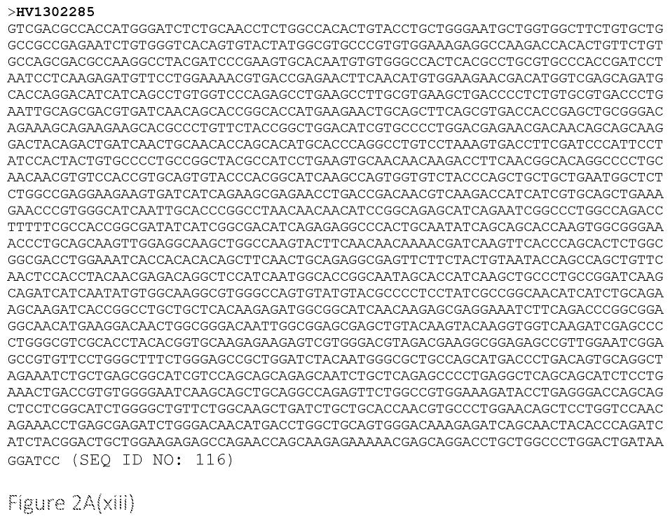

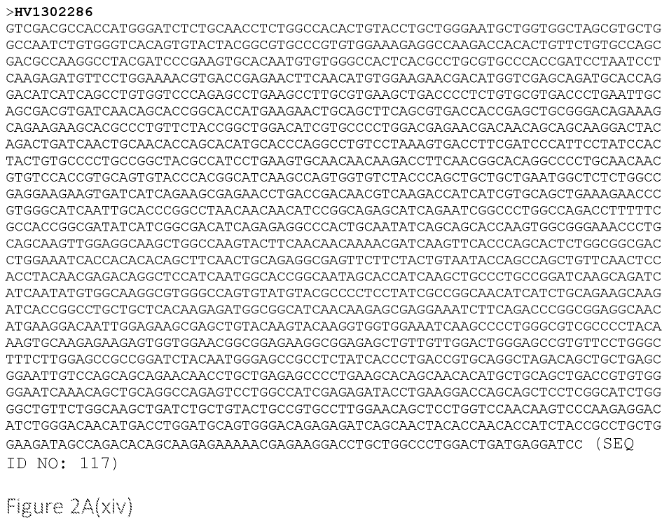

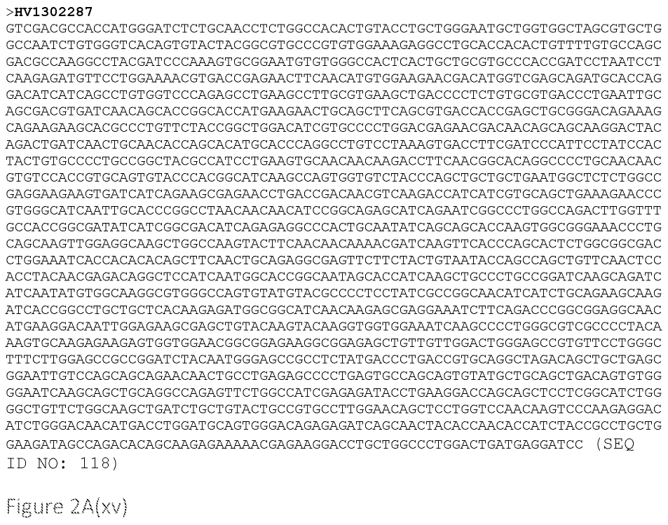

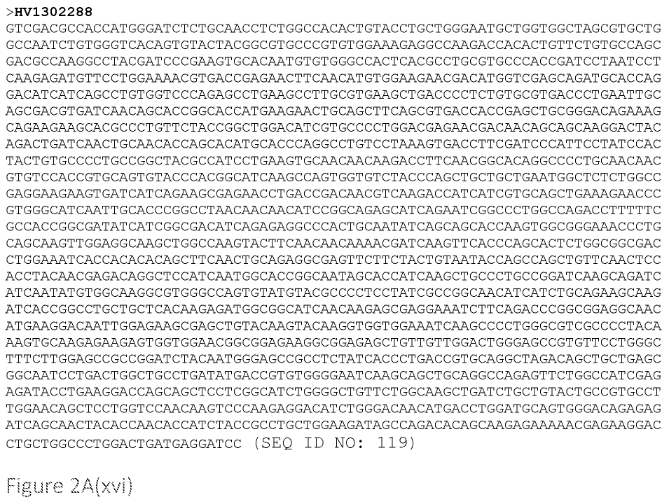

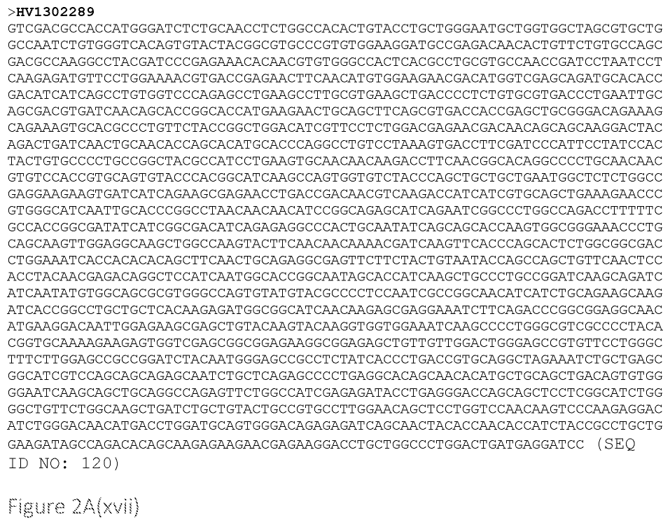

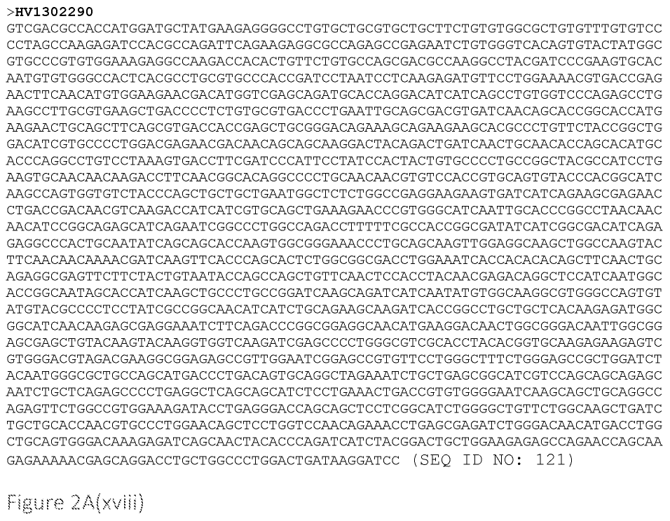

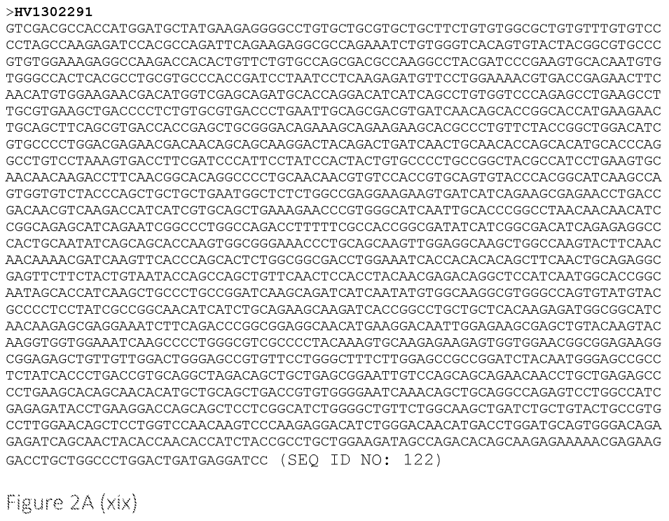

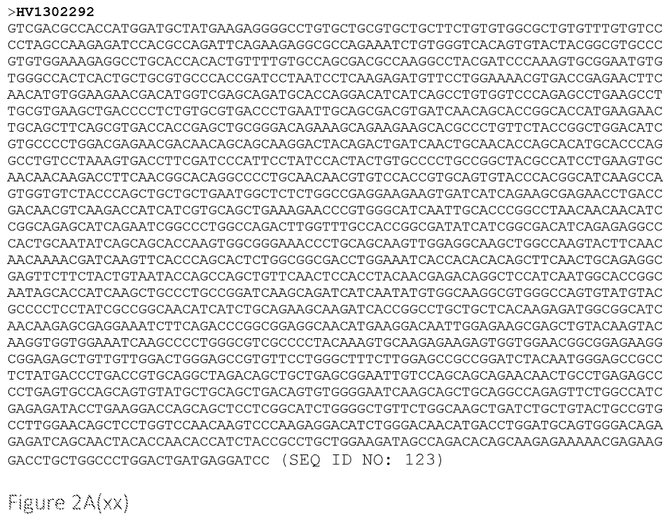

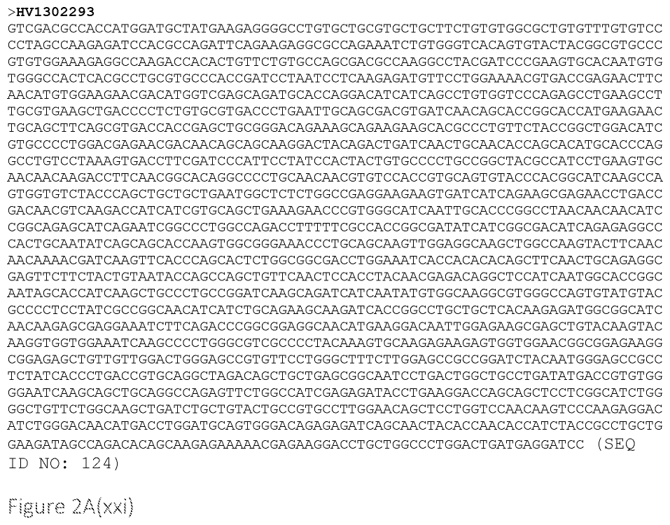

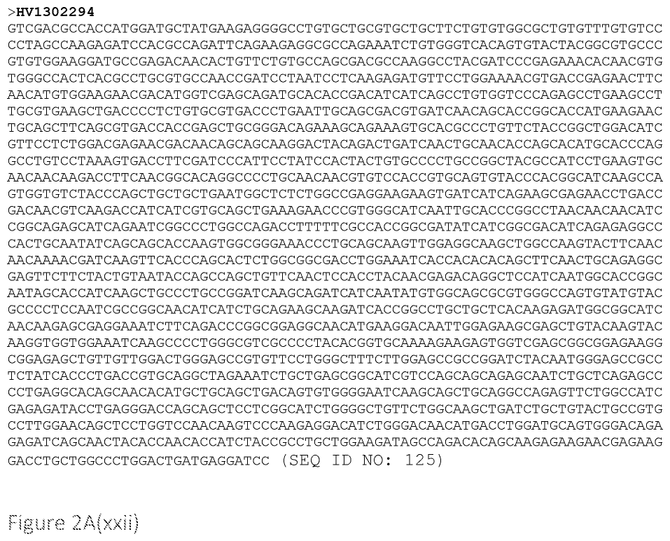

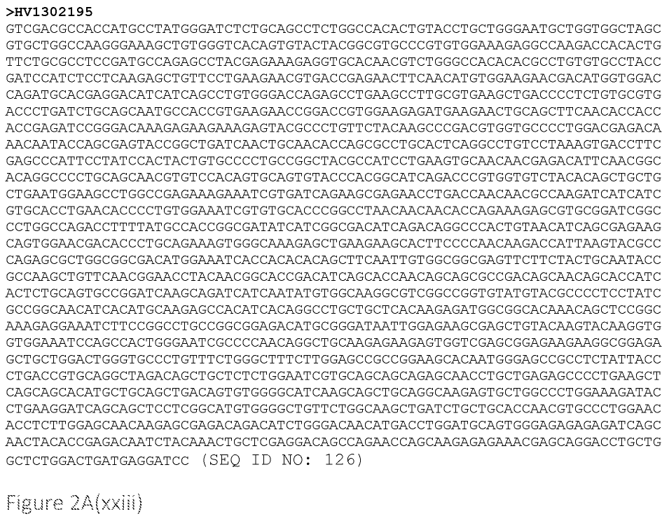

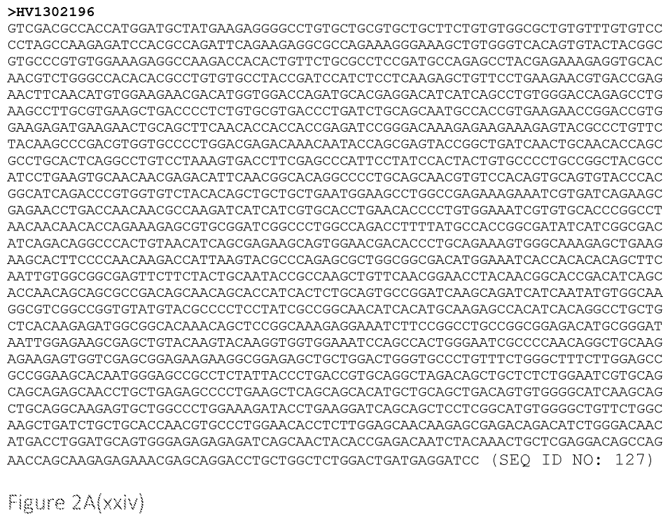

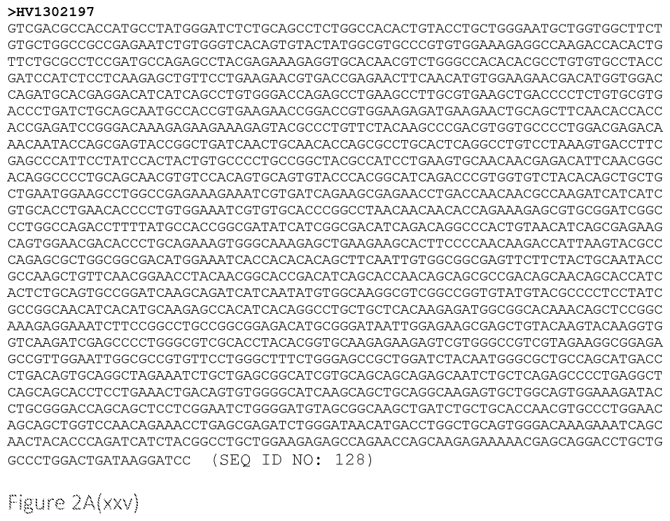

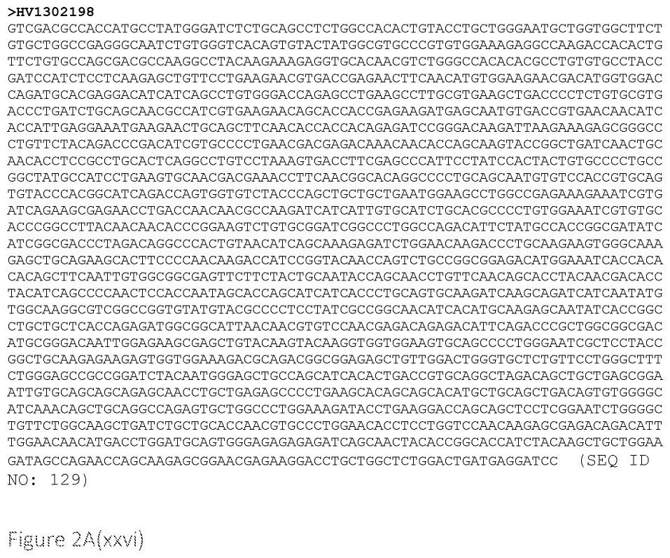

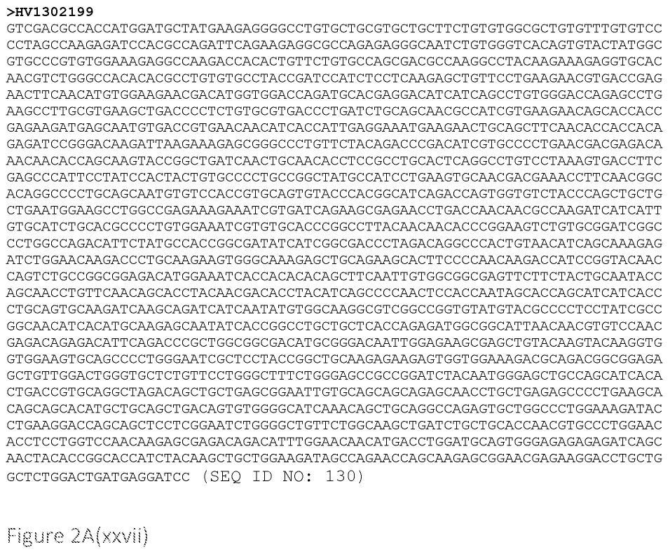

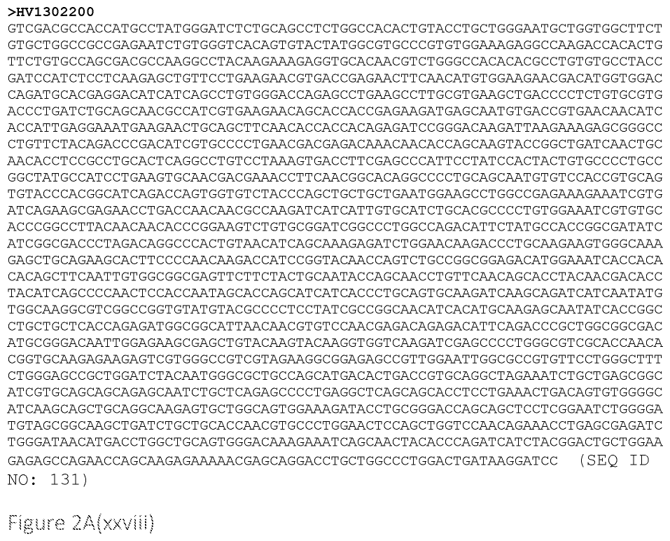

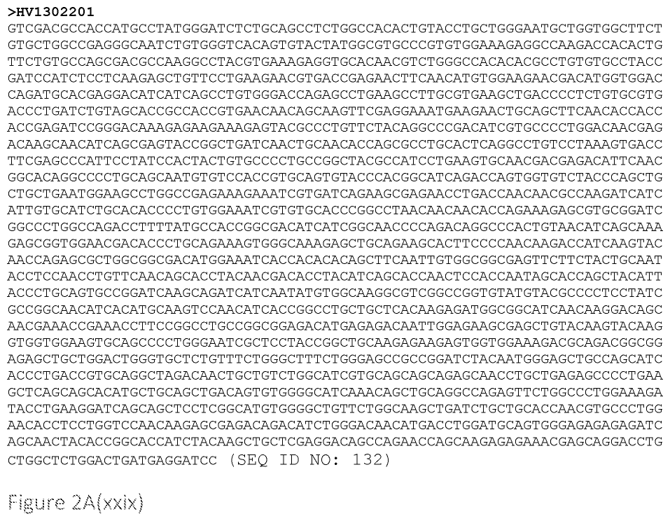

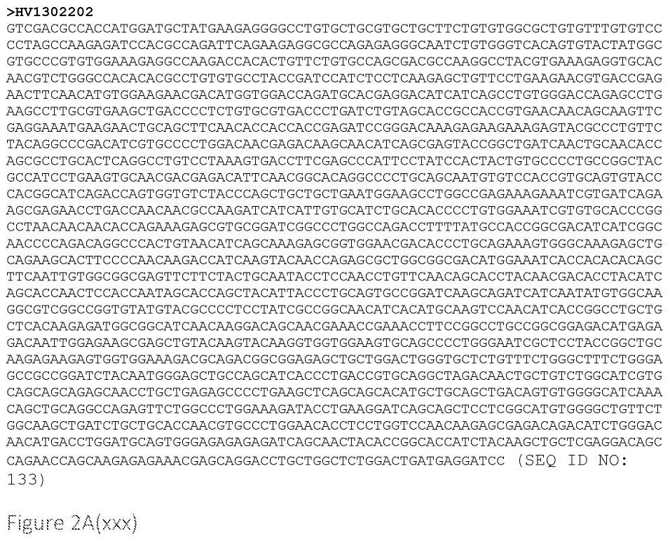

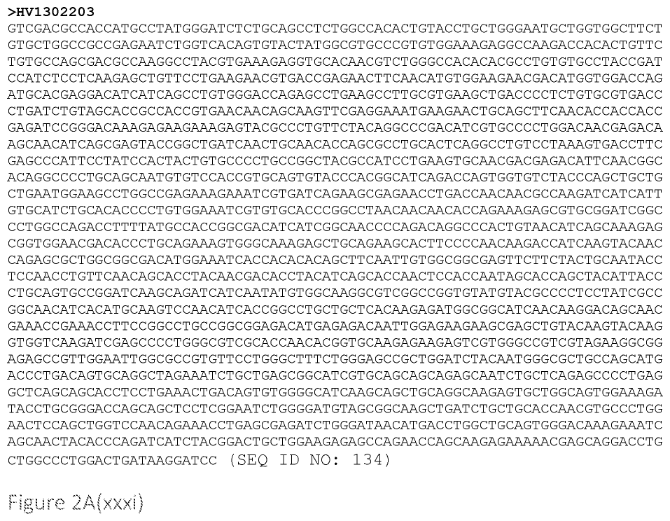

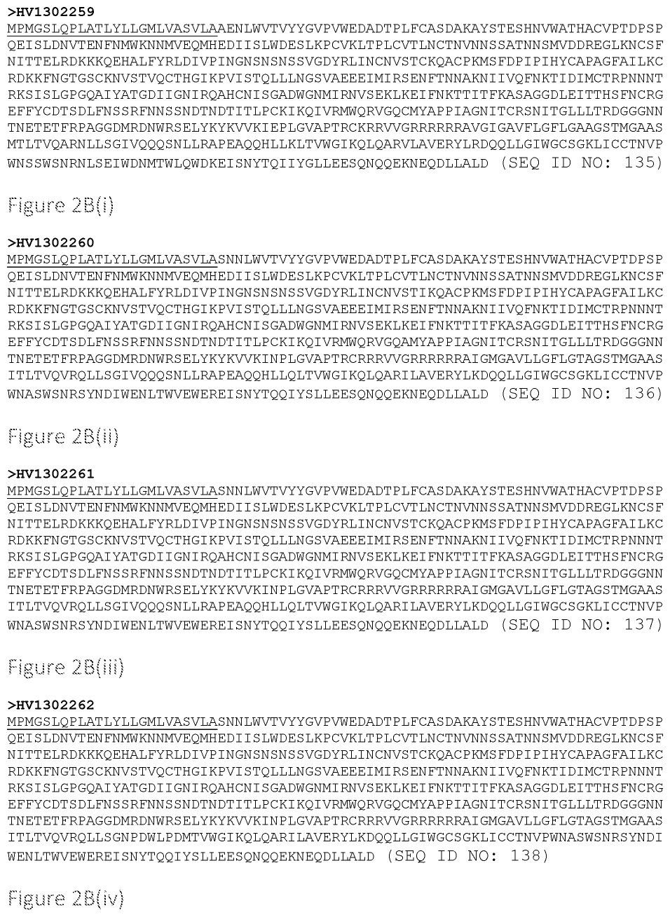

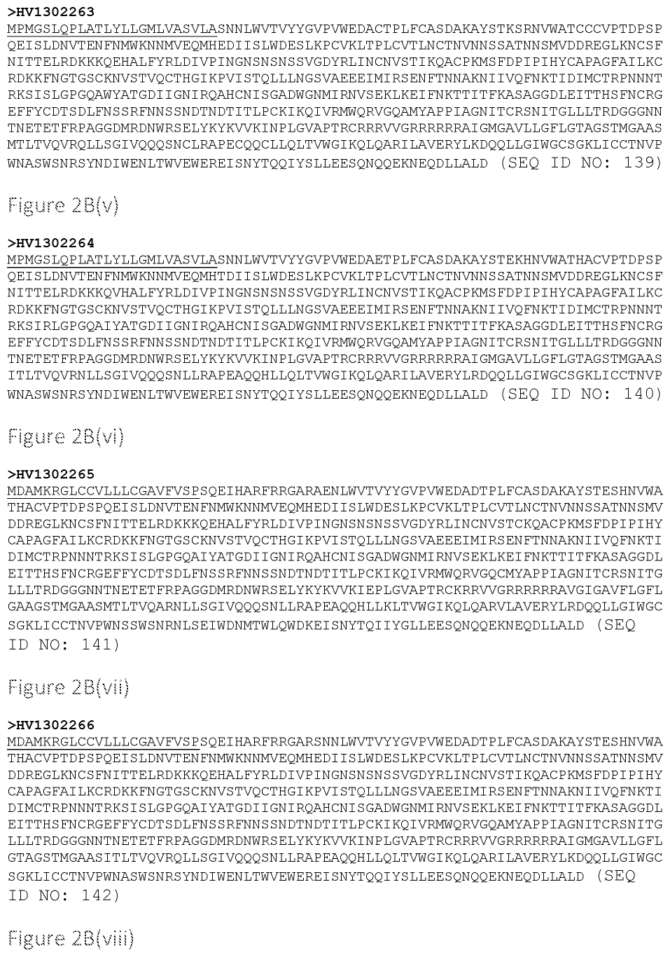

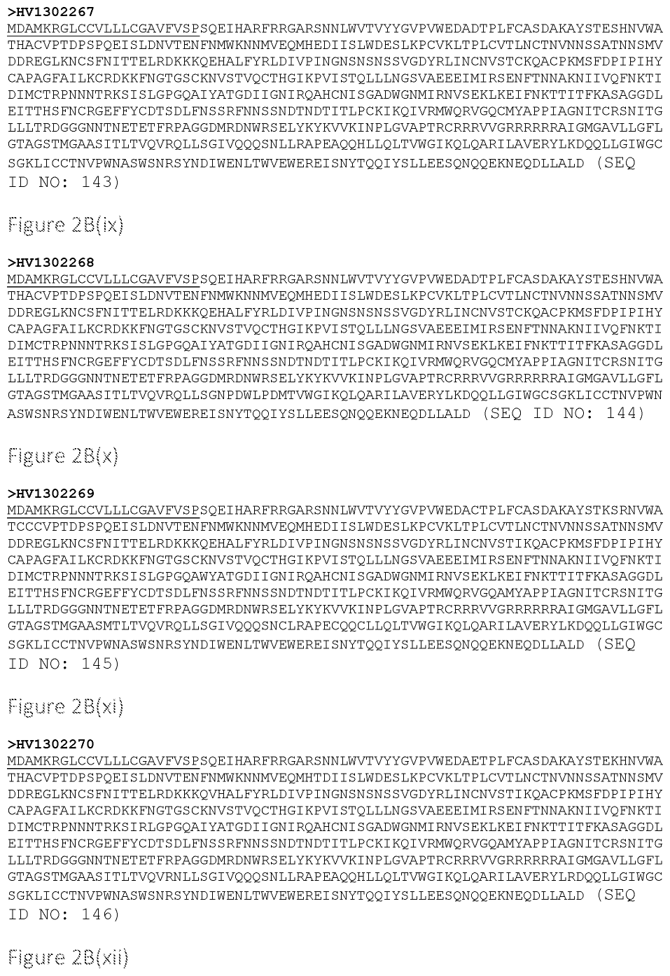

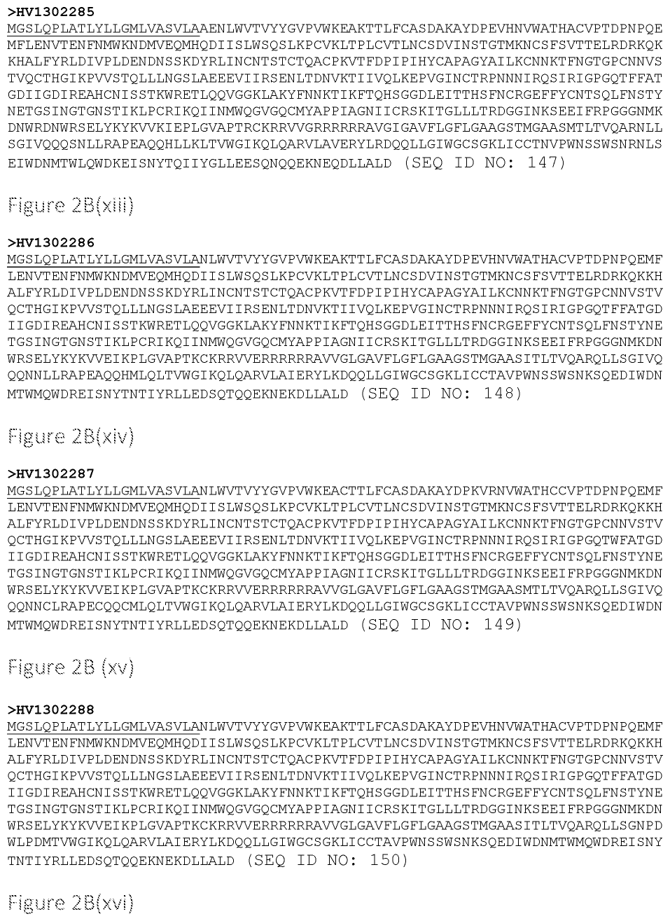

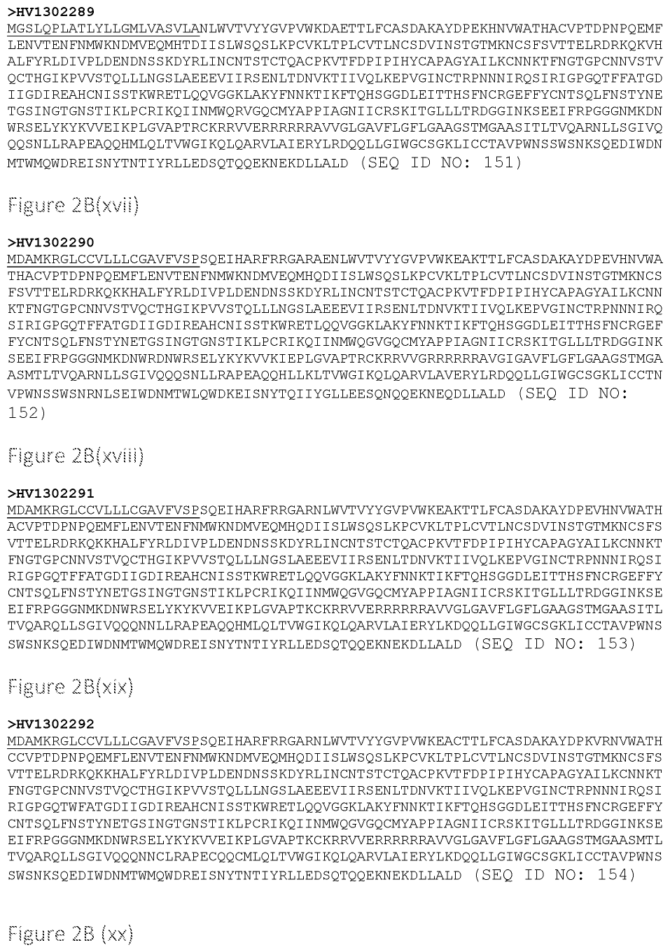

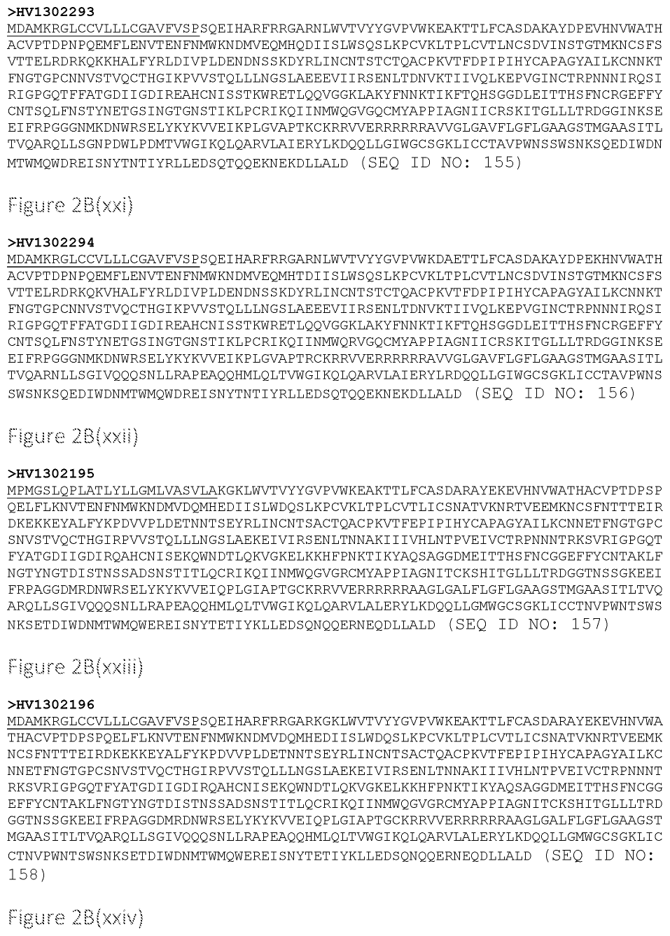

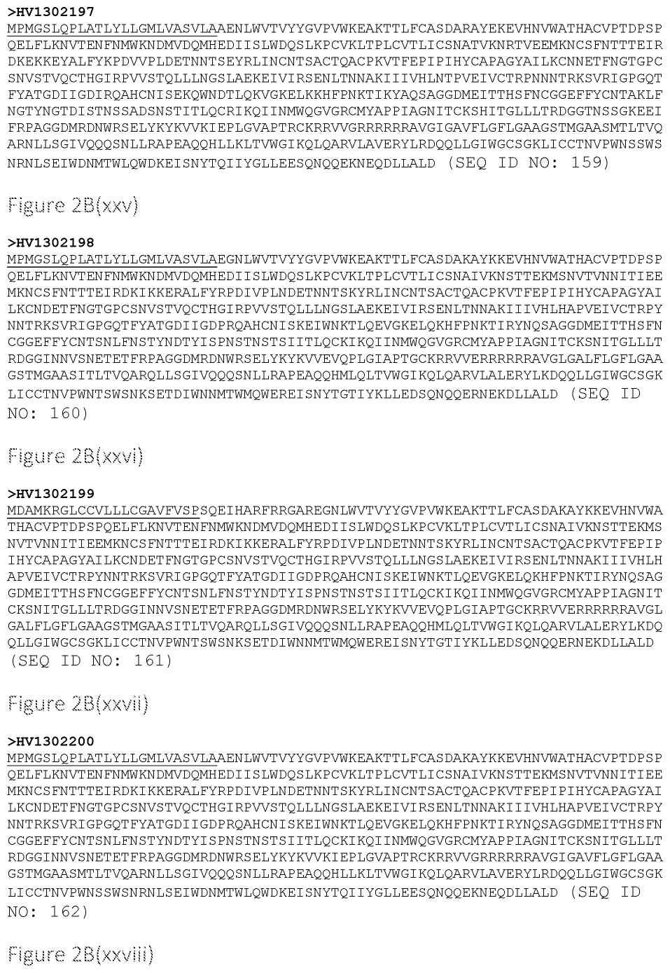

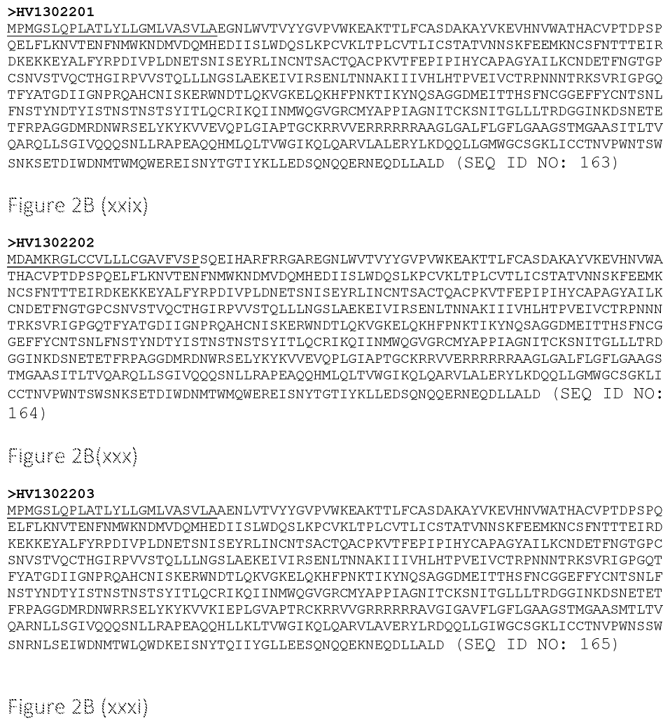

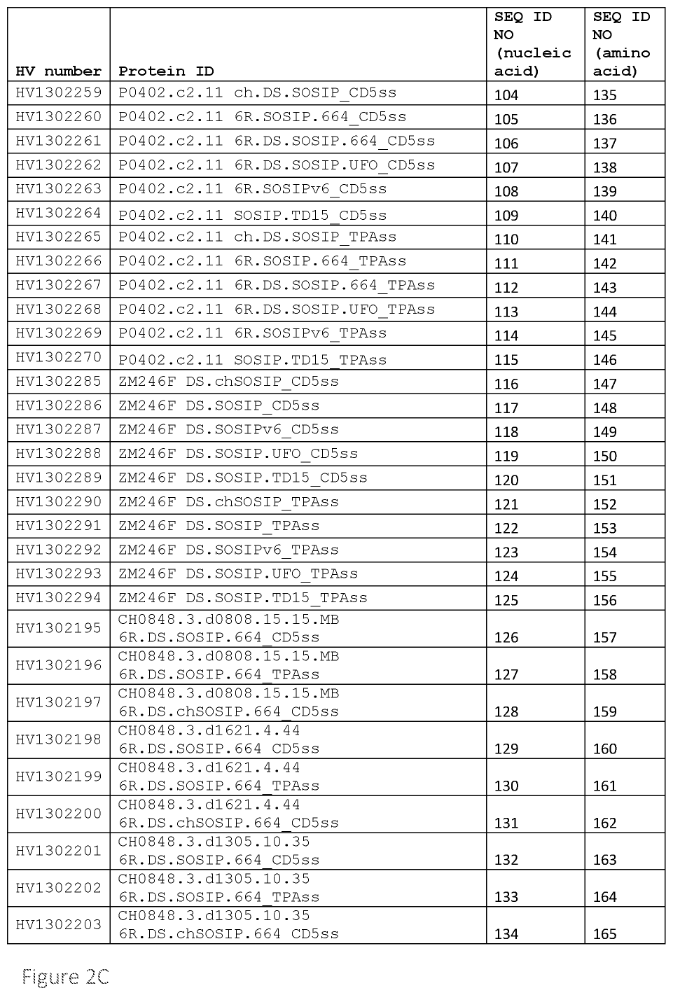

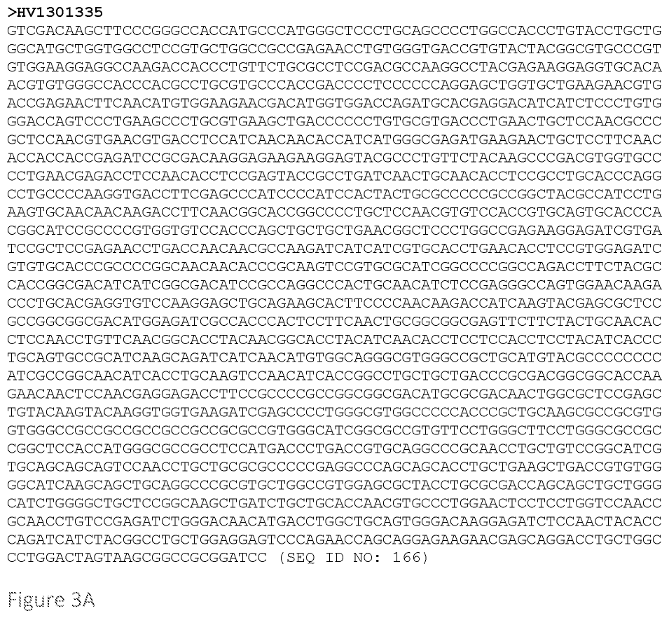

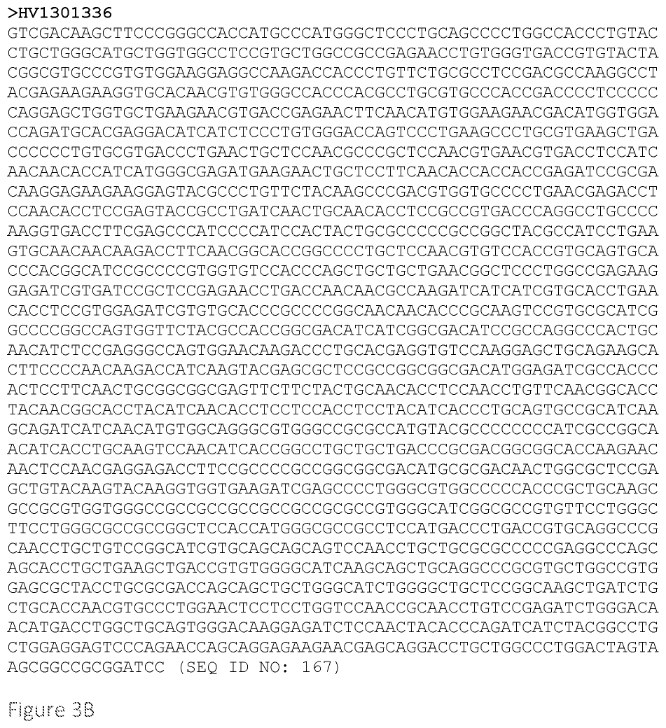

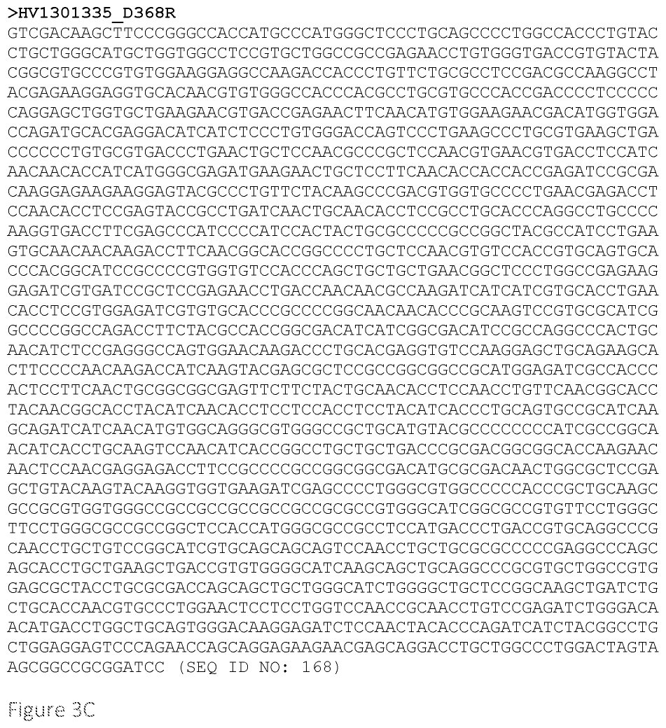

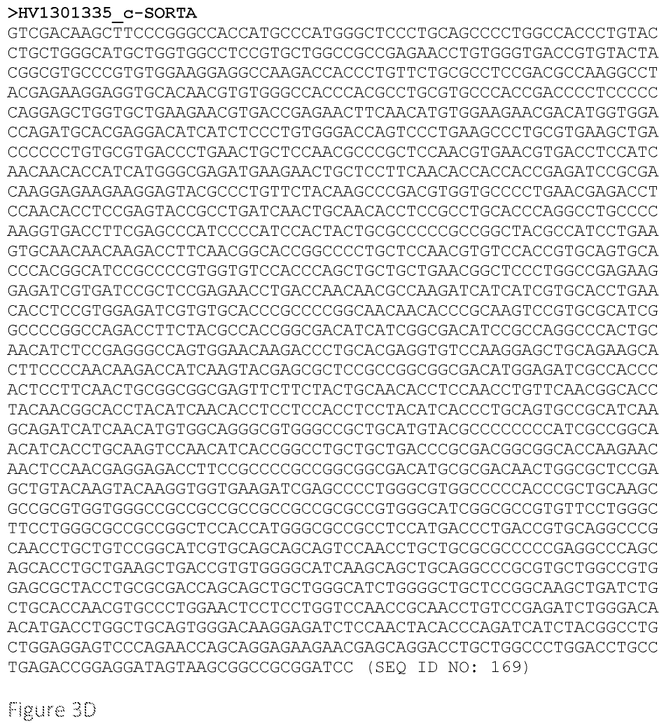

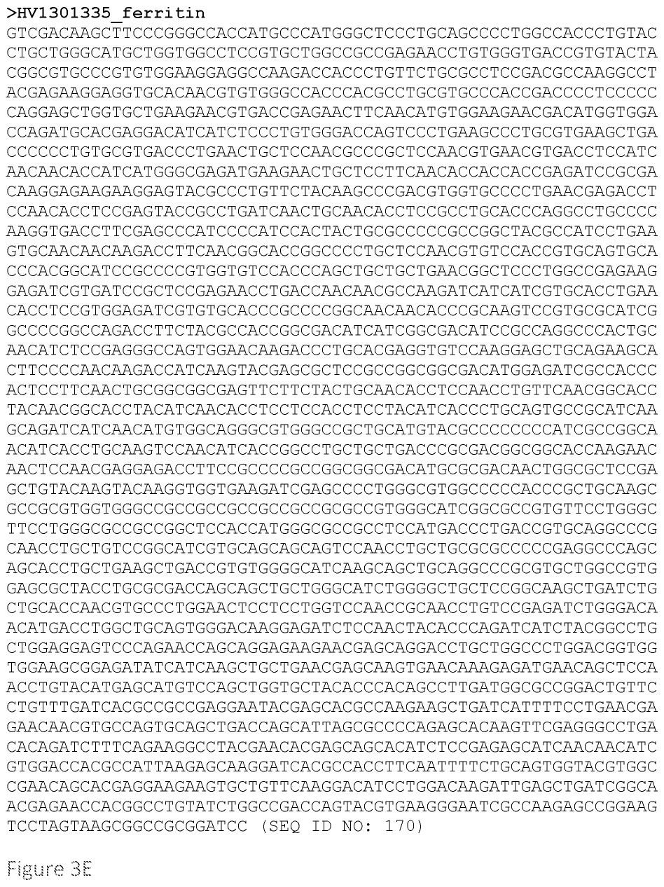

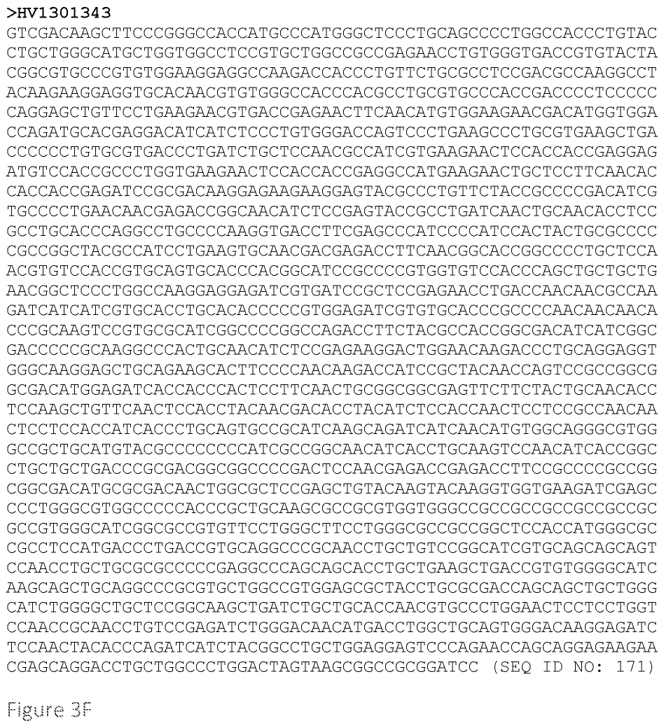

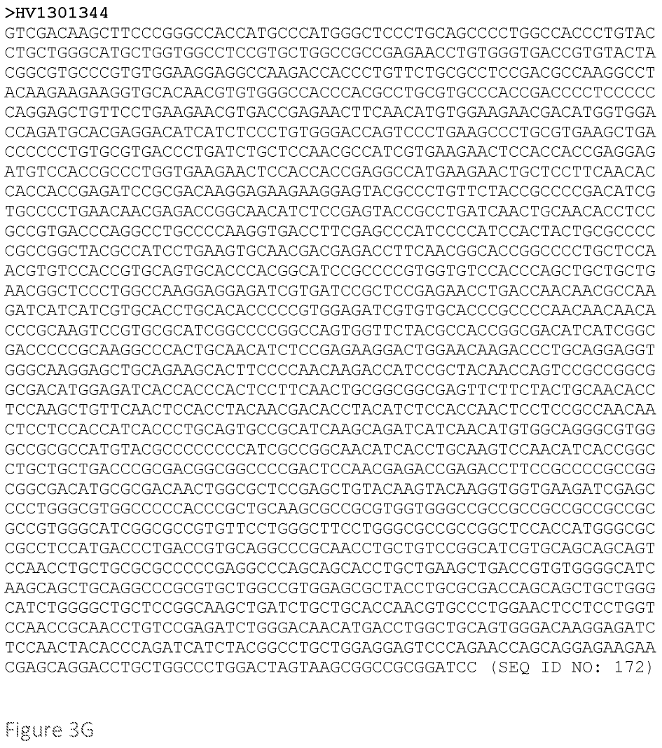

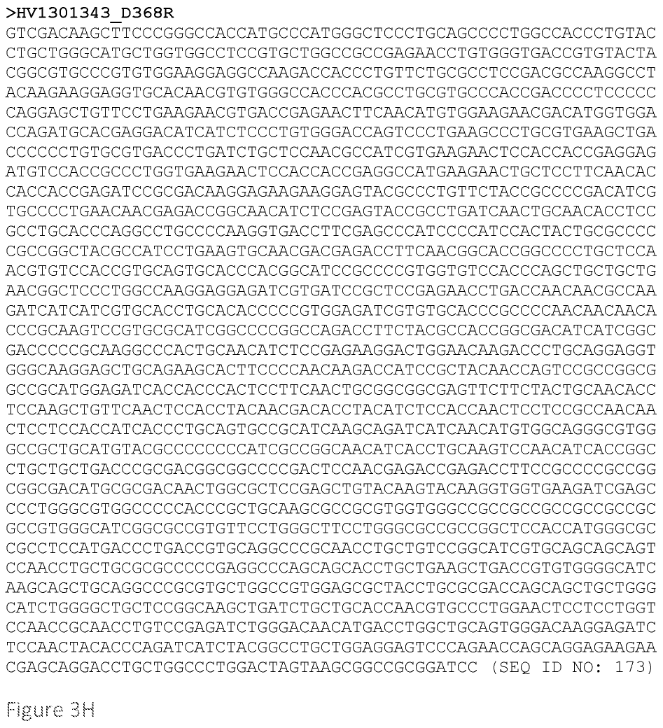

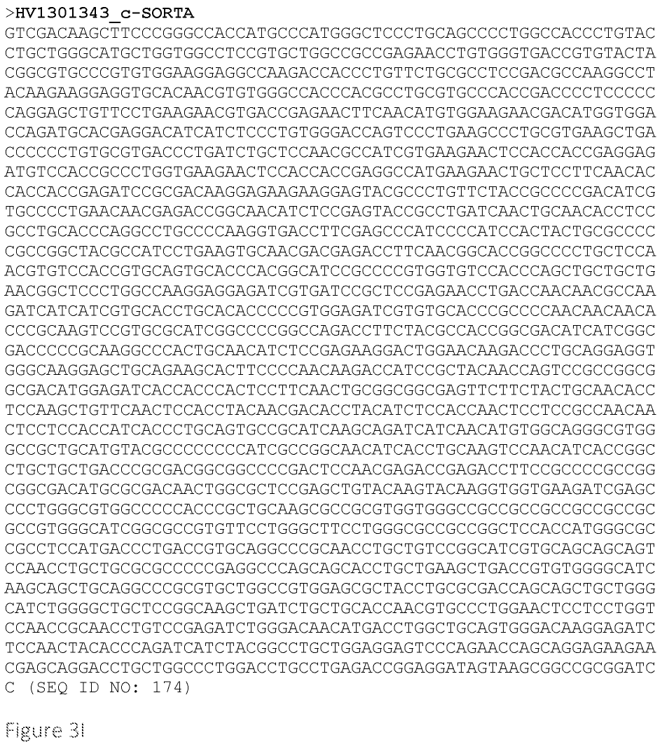

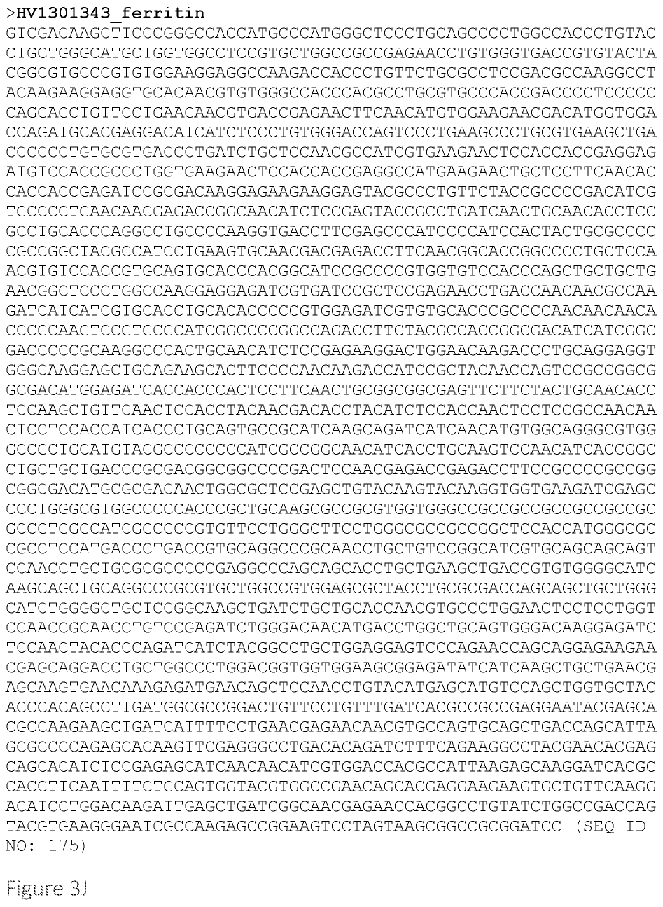

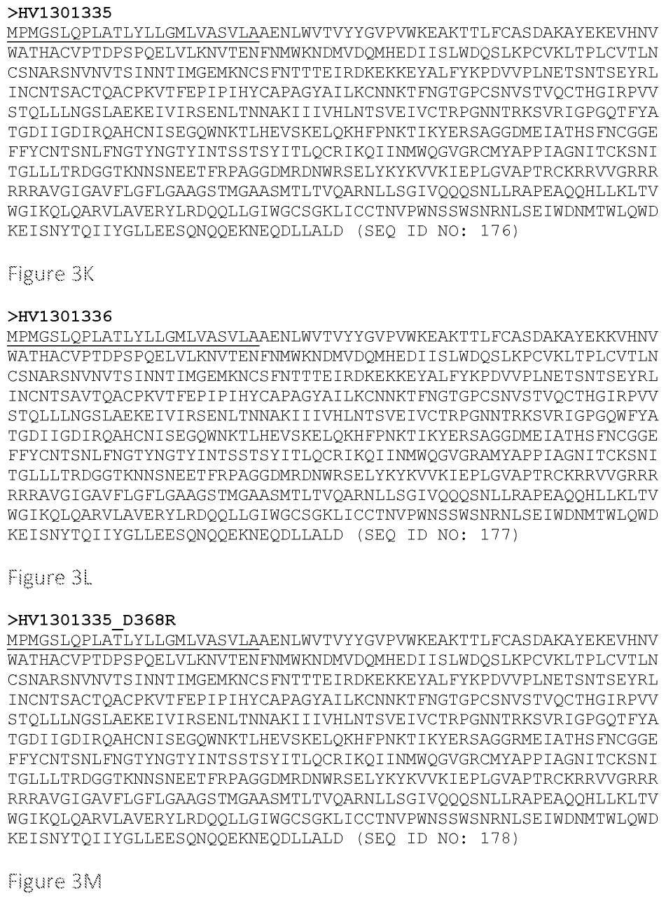

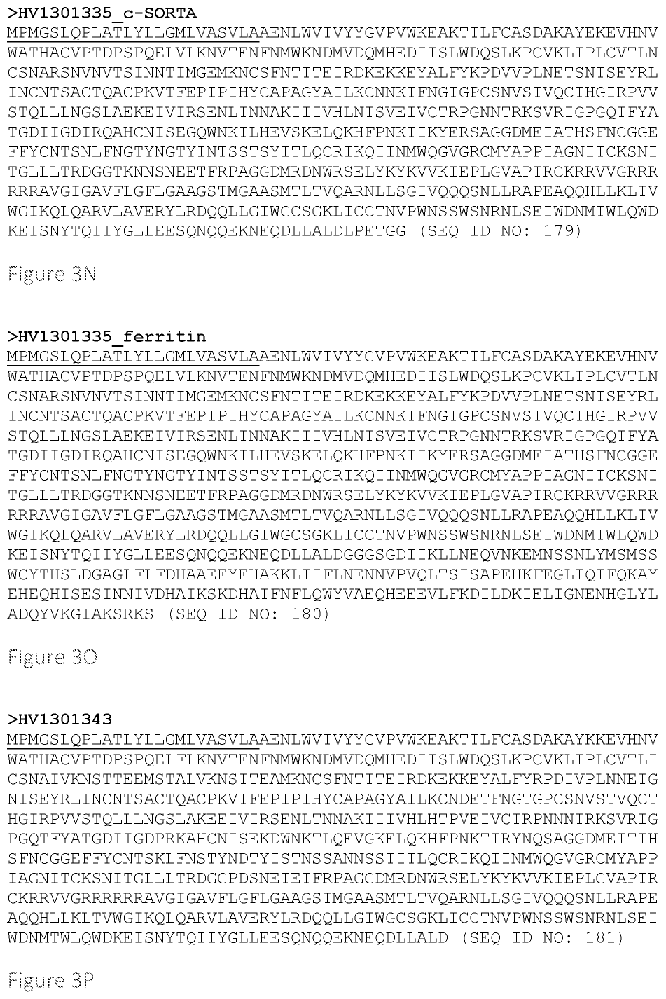

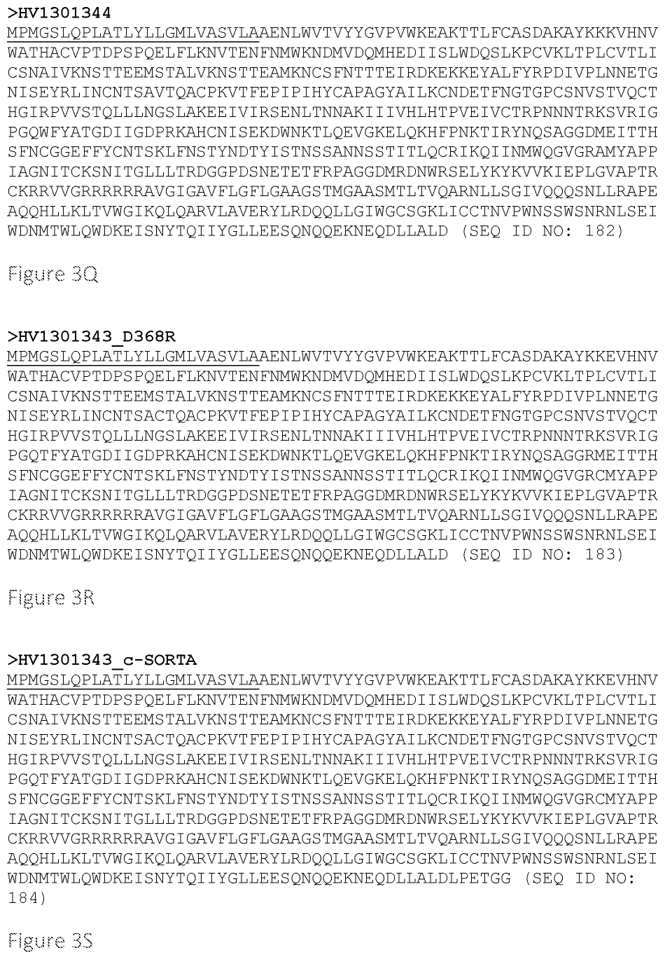

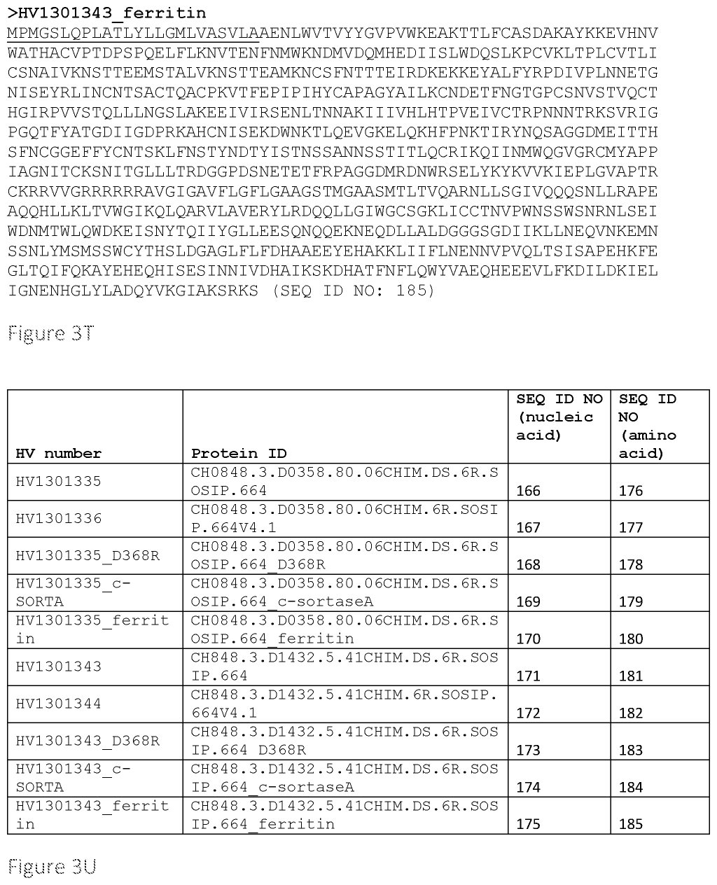

In certain embodiments the envelope is any of the forms of HIV-1 envelope. In certain embodiments the envelope is gp120, gp140, gp145 (i.e. with a transmembrane domain), or gp150. In certain embodiments, gp140 is designed to form a stable trimer. See Tables 3-9, FIGS. 1-3 and 20 for non-limiting examples of sequence designs. In certain embodiments envelope protomers form a trimer which is not a SOSIP timer. In certain embodiment the trimer is a SOSIP based trimer wherein each protomer comprises additional modifications. In certain embodiments, envelope trimers are recombinantly produced. In certain embodiments, envelope trimers are purified from cellular recombinant fractions by antibody binding and reconstituted in lipid comprising formulations. See for example WO2015/127108 titled “Trimeric HIV-1 envelopes and uses thereof” and WO2017/151801 the content of each of which is herein incorporated by reference in its entirety. In certain embodiments the envelopes of the invention are engineered and comprise non-naturally occurring modifications.

In certain embodiments, the envelope is in a liposome. In certain embodiments the envelope comprises a transmembrane domain with a cytoplasmic tail, wherein the transmembrane domain is embedded in a liposome. In certain embodiments, the nucleic acid comprises a nucleic acid sequence which encodes a gp120, gp140, gp145, gp150, or gp160.

In certain embodiments, where the nucleic acids are operably linked to a promoter and inserted in a vector, the vector is any suitable vector. Non-limiting examples include, VSV, replicating rAdenovirus type 4, MVA, Chimp adenovirus vectors, pox vectors, and the like. In certain embodiments, the nucleic acids are administered in NanoTaxi block polymer nanospheres. In certain embodiments, the composition and methods comprise an adjuvant. Non-limiting examples include, 3M052, AS01 B, AS01 E, gla/SE, alum, Poly I poly C (poly IC), polyIC/long chain (LC) TLR agonists, TLR7/8 and 9 agonists, or a combination of TLR7/8 and TLR9 agonists (see Moody et al. (2014) J. Virol. March 2014 vol. 88 no. 6 3329-3339), or any other adjuvant. Non-limiting examples of TLR7/8 agonist include TLR7/8 ligands, Gardiquimod, Imiquimod and R848 (resiquimod). A non-limiting embodiment of a combination of TLR7/8 and TLR9 agonist comprises R848 and oCpG in STS (see Moody et al. (2014) J. Virol. March 2014 vol. 88 no. 6 3329-3339).

In non-limiting embodiments, the adjuvant is an LNP. See e.g., without limitation Shirai et al. “Lipid Nanoparticle Acts as a Potential Adjuvant for Influenza Split Vaccine without Inducing Inflammatory Responses” Vaccines 2020, 8, 433; doi:10.3390/vaccines8030433, published 3 Aug. 2020.

In non-limiting embodiments, LNPs used as adjuvants for protein or mRNA compositions are composed of an ionizable lipid, cholesterol, lipid conjugated with polyethylene glycol, and a helper lipid. Non-limiting embodiments include LNPs without polyethylene glycol.

In certain aspects the invention provides a cell comprising a nucleic acid encoding any one of the envelopes of the invention suitable for recombinant expression. In certain aspects, the invention provides a clonally derived population of cells encoding any one of the envelopes of the invention suitable for recombinant expression. In certain aspects, the invention provides a stable pool of cells encoding any one of the envelopes of the invention suitable for recombinant expression.

In certain aspects, the invention provides a recombinant HIV-1 envelope polypeptide as described here, wherein the polypeptide is a non-naturally occurring protomer designed to form an envelope trimer. The invention also provides nucleic acids encoding these recombinant polypeptides. Non-limiting examples of amino acids and nucleic acid of such protomers are disclosed herein.

In certain aspects the invention provides a recombinant trimer comprising three identical protomers of an envelope. In certain aspects the invention provides an immunogenic composition comprising the recombinant trimer and a carrier, wherein the trimer comprises three identical protomers of an HIV-1 envelope as described herein. In certain aspects the invention provides an immunogenic composition comprising nucleic acid encoding these recombinant HIV-1 envelope and a carrier.

Described herein are nucleic and amino acids sequences of HIV-1 envelopes. The sequences for use as immunogens are in any suitable form. In certain embodiments, the described HIV-1 envelope sequences are gp160s. In certain embodiments, the described HIV-1 envelope sequences are gp120s. Other sequences, for example but not limited to stable SOSIP trimer designs, gp145s, gp140s, both cleaved and uncleaved, gp140 Envs with the deletion of the cleavage (C) site, fusion (F) and immunodominant (I) region in gp41—named as gp140ΔCFI (gp140CFI), gp140 Envs with the deletion of only the cleavage (C) site and fusion (F) domain—named as gp140ΔCF (gp140CF), gp140 Envs with the deletion of only the cleavage (C)—named gp140ΔC (gp140C) (See e.g. Liao et al. Virology 2006, 353, 268-282), gp150s, gp41s, which can be readily derived from the nucleic acid and amino acid gp160 sequences. In certain embodiments the nucleic acid sequences are codon optimized for optimal expression in a host cell, for example a mammalian cell, a rBCG cell or any other suitable expression system.

An HIV-1 envelope has various structurally defined fragments/forms: gp160; gp140—including cleaved gp140 and uncleaved gp140 (gp140C), gp140CF, or gp140CFI; gp120 and gp41. A skilled artisan appreciates that these fragments/forms are defined not necessarily by their crystal structure, but by their design and bounds within the full length of the gp160 envelope. While the specific consecutive amino acid sequences of envelopes from different strains are different, the bounds and design of these forms are well known and characterized in the art.

For example, it is well known in the art that during its transport to the cell surface, the gp160 polypeptide is processed and proteolytically cleaved to gp120 and gp41 proteins. Cleavages of gp160 to gp120 and gp41 occurs at a conserved cleavage site “REKR” (SEQ ID NO:4). See Chakrabarti et al. Journal of Virology vol. 76, pp. 5357-5368 (2002) see for example FIG. 1, and second paragraph in the Introduction on p. 5357; Binley et al. Journal of Virology vol. 76, pp. 2606-2616 (2002) for example at Abstract; Gao et al. Journal of Virology vol. 79, pp. 1154-1163 (2005); Liao et al. Virology vol. 353(2): 268-282 (2006).

The role of the furin cleavage site was well understood both in terms of improving cleavage efficiency, see Binley et al. supra, and eliminating cleavage, see Bosch and Pawlita, Virology 64 (5):2337-2344 (1990); Guo et al. Virology 174: 217-224 (1990); McCune et al. Cell 53:55-67 (1988); Liao et al. J Virol. April; 87(8):4185-201 (2013).

Likewise, the design of gp140 envelope forms is also well known in the art, along with the various specific changes which give rise to the gp140C (uncleaved envelope), gp140CF and gp140CFI forms. Envelope gp140 forms are designed by introducing a stop codon within the gp41 sequence. See Chakrabarti et al. at FIG. 1.

Envelope gp140C refers to a gp140 HIV-1 envelope design with a functional deletion of the cleavage (C) site, so that the gp140 envelope is not cleaved at the furin cleavage site. The specification describes cleaved and uncleaved forms, and various furin cleavage site modifications that prevent envelope cleavage are known in the art. In some embodiments of the gp140C form, two of the R residues in and near the furin cleavage site are changed to E, e.g., RRVVEREKR (SEQ ID NO:5) is changed to ERVVEREKE (SEQ ID NO:6), and is one example of an uncleaved gp140 form. Another example is the gp140C form which has the REKR site (SEQ ID NO:4) changed to SEKS (SEQ ID NO:7). See supra for references.

Envelope gp140CF refers to a gp140 HIV-1 envelope design with a deletion of the cleavage (C) site and fusion (F) region. Envelope gp140CFI refers to a gp140 HIV-1 envelope design with a deletion of the cleavage (C) site, fusion (F) and immunodominant (I) region in gp41. See Chakrabarti et al. Journal of Virology vol. 76, pp. 5357-5368 (2002) at for example FIG. 1, and Second paragraph in the Introduction on p. 5357; Binley et al. Journal of Virology vol. 76, pp. 2606-2616 (2002) for example at Abstract; Gao et al. Journal of Virology vol. 79, pp. 1154-1163 (2005); Liao et al. Virology vol. 353(2): 268-282 (2006).

In certain embodiments, the envelope design in accordance with the present invention involves deletion of residues (e.g., 5-11, 5, 6, 7, 8, 9, 10, or 11 amino acids) at the N-terminus. For delta N-terminal design, amino acid residues ranging from 4 residues or even fewer to 14 residues or even more are deleted. These residues are between the maturation (signal peptide, usually ending with CXX, wherein X can be any amino acid) and “VPVXXXX . . . ”. In case of CH505 T/F Env as an example, 8 amino acids (italicized and underlined in the below sequence) were deleted: MRVMGIQRNYPQWWIWSMLGFWMLMICNGMWVTVYYGVPVWKEAKTTLFCASDA KAYEKEVHNVWATHACVPTDPNPQE . . . (SEQ ID NO:8) (rest of envelope sequence is indicated as “ . . . ”). In other embodiments, the delta N-design described for CH505 T/F envelope can be used to make delta N-designs of other envelopes. In certain embodiments, the invention relates generally to an HIV-1 envelope immunogen, gp160, gp120, or gp140, without an N-terminal Herpes Simplex gD tag substituted for amino acids of the N-terminus of gp120, with an HIV leader sequence (or other leader sequence), and without the original about 4 to about 25, for example 4, 5, 6, 7, 8, 9, 10, 11, 12, 13, 14, 15, 16, 17, 18, 19, 20, 21, 22, 23, 24, 25 amino acids of the N-terminus of the envelope (e.g. gp120). See WO2013/006688, e.g. at pages 10-12, the contents of which publication is hereby incorporated by reference in its entirety.

The general strategy of deletion of N-terminal amino acids of envelopes results in proteins, for example gp120s, expressed in mammalian cells that are primarily monomeric, as opposed to dimeric, and, therefore, solves the production and scalability problem of commercial gp120 Env vaccine production. In other embodiments, the amino acid deletions at the N-terminus results in increased immunogenicity of the envelopes.

In certain aspects, the invention provides composition and methods which use a selection of Envs, as gp120s, gp140s cleaved and uncleaved, gp145s, gp150s and gp160s, stabilized and/or multimerized trimers, as proteins, DNAs, RNAs, or any combination thereof, administered as primes and boosts to elicit immune response. Envs as proteins could be co-administered with nucleic acid vectors containing Envs to amplify antibody induction. In certain embodiments, the compositions and methods include any immunogenic HIV-1 sequences to give the best coverage for T cell help and cytotoxic T cell induction. In certain embodiments, the compositions and methods include mosaic and/or consensus HIV-1 genes to give the best coverage for T cell help and cytotoxic T cell induction. In certain embodiments, the compositions and methods include mosaic group M and/or consensus genes to give the best coverage for T cell help and cytotoxic T cell induction. In some embodiments, the mosaic genes are any suitable gene from the HIV-1 genome. In some embodiments, the mosaic genes are Env genes, Gag genes, Pol genes, Nef genes, or any combination thereof. See e.g. U.S. Pat. No. 7,951,377. In some embodiments the mosaic genes are bivalent mosaics. In some embodiments the mosaic genes are trivalent. In some embodiments, the mosaic genes are administered in a suitable vector with each immunization with Env gene inserts in a suitable vector and/or as a protein. In some embodiments, the mosaic genes, for example as bivalent mosaic Gag group M consensus genes, are administered in a suitable vector, for example but not limited to HSV2, would be administered with each immunization with Env gene inserts in a suitable vector, for example but not limited to HSV-2.

In certain aspects the invention provides compositions and methods of Env genetic immunization either alone or with Env proteins to recreate the swarms of evolved viruses that have led to bnAb induction. Nucleotide-based vaccines offer a flexible vector format to immunize against virtually any protein antigen. Currently, two types of genetic vaccination are available for testing—DNAs and mRNAs.

In certain aspects the invention contemplates using immunogenic compositions wherein immunogens are delivered as DNA. See Graham B S, Enama M E, Nason M C, Gordon I J, Peel S A, et al. (2013) DNA Vaccine Delivered by a Needle-Free Injection Device Improves Potency of Priming for Antibody and CD8+ T-Cell Responses after rAd5 Boost in a Randomized Clinical Trial. PLoS ONE 8(4): e59340, page 9. Various technologies for delivery of nucleic acids, as DNA and/or RNA, so as to elicit immune response, both T-cell and humoral responses, are known in the art and are under developments. In certain embodiments, DNA can be delivered as naked DNA. In certain embodiments, DNA is formulated for delivery by a gene gun. In certain embodiments, DNA is administered by electroporation, or by a needle-free injection technology, for example but not limited to Biojector® device. In certain embodiments, the DNA is inserted in vectors. The DNA is delivered using a suitable vector for expression in mammalian cells. In certain embodiments the nucleic acids encoding the envelopes are optimized for expression. In certain embodiments DNA is optimized, e.g. codon optimized, for expression. In certain embodiments the nucleic acids are optimized for expression in vectors and/or in mammalian cells. In non-limiting embodiments these are bacterially derived vectors, adenovirus based vectors, rAdenovirus (e.g. Barouch D H, et al. Nature Med. 16: 319-23, 2010), recombinant mycobacteria (e.g. rBCG or M. smegmatis) (Yu, J S et al. Clinical Vaccine Immunol. 14: 886-093, 2007; ibid 13: 1204-11, 2006), and recombinant vaccinia type of vectors (Santra S. Nature Med. 16: 324-8, 2010), for example but not limited to ALVAC, replicating (Kibler K V et al., PLoS One 6: e25674, 2011 nov 9.) and non-replicating (Perreau M et al. J. virology 85: 9854-62, 2011) NYVAC, modified vaccinia Ankara (MVA)), adeno-associated virus, Venezuelan equine encephalitis (VEE) replicons, Herpes Simplex Virus vectors, and other suitable vectors.

In certain aspects the invention contemplates using immunogenic compositions wherein immunogens are delivered as DNA or RNA in suitable formulations. Various technologies which contemplate using DNA or RNA, or may use complexes of nucleic acid molecules and other entities to be used in immunization. In certain embodiments, DNA or RNA is administered as nanoparticles consisting of low dose antigen-encoding DNA formulated with a block copolymer (amphiphilic block copolymer 704). See Cany et al., Journal of Hepatology 2011 vol. 54 j 115-121; Arnaoty et al., Chapter 17 in Yves Bigot (ed.), Mobile Genetic Elements: Protocols and Genomic Applications, Methods in Molecular Biology, vol. 859, pp 293-305 (2012); Arnaoty et al. (2013) Mol Genet Genomics. 2013 August; 288(7-8):347-63. Nanocarrier technologies called Nanotaxi® for immunogenic macromolecules (DNA, RNA, Protein) delivery are under development. See for example technologies developed by incellart.

In certain aspects, the invention provides nucleic acids comprising sequences encoding envelopes of the invention. In certain embodiments, the nucleic acids are DNAs. In certain embodiments, the nucleic acids are mRNAs. In certain aspects, the invention provides expression vectors comprising the nucleic acids of the invention.

In certain aspects, the invention provides a pharmaceutical composition comprising mRNAs encoding the inventive immunogens. In certain embodiments, these are optionally formulated in lipid nanoparticles (LNPs). In certain embodiments, the mRNAs are modified. Modifications include without limitations modified ribonucleotides, poly-A tail, 5′ cap.

In certain aspects the invention provides nucleic acids encoding the inventive envelopes. In non-limiting embodiments, the nucleic acids are mRNA, modified or unmodified, suitable for use any use, e.g. but not limited to use as pharmaceutical compositions. In certain embodiments, the nucleic acids are formulated in lipid, such as but not limited to LNPs.

In some embodiments the immunogens are administered as nucleic acids, including but not limited to mRNAs which could be modified and/or unmodified. See US Pub 20180028645A1, US Pub 20090286852, US Pub 20130111615, US Pub 20130197068, US Pub 20130261172, US Pub 20150038558, US Pub 20160032316, US Pub 20170043037, US Pub 20170327842, U.S. Pat. Nos. 10,006,007, 9,371,511, 9,012,219, US Pub 20180265848, US Pub 20170327842, US Pub 20180344838A1 at least at paragraphs [0260]-[0281], WO/2017/182524 for non-limiting embodiments of chemical modifications, wherein the content of each of which is hereby incorporated by reference in its entirety.

mRNAs delivered in LNP formulations have advantages over non-LNPs formulations. See US Pub 20180028645A1, WO/2018/081638, WO/2016/176330, wherein the content of each of which is incorporated by reference in its entirety.

In certain embodiments the nucleic acid encoding an envelope is operably linked to a promoter inserted an expression vector. In certain aspects the compositions comprise a suitable carrier. In certain aspects the compositions comprise a suitable adjuvant.

In certain aspects the invention provides an expression vector comprising any of the nucleic acid sequences of the invention, wherein the nucleic acid is operably linked to a promoter. In certain aspects the invention provides an expression vector comprising a nucleic acid sequence encoding any of the polypeptides of the invention, wherein the nucleic acid is operably linked to a promoter. In certain embodiments, the nucleic acids are codon optimized for expression in a mammalian cell, in vivo or in vitro. In certain aspects the invention provides nucleic acids comprising any one of the nucleic acid sequences of invention. In certain aspects the invention provides nucleic acids consisting essentially of any one of the nucleic acid sequences of invention. In certain aspects the invention provides nucleic acids consisting of any one of the nucleic acid sequences of invention. In certain embodiments the nucleic acid of the invention, is operably linked to a promoter and is inserted in an expression vector. In certain aspects the invention provides an immunogenic composition comprising the expression vector.

In certain aspects the invention provides a composition comprising at least one of the nucleic acid sequences of the invention. In certain aspects the invention provides a composition comprising any one of the nucleic acid sequences of invention. In certain aspects the invention provides a composition comprising at least one nucleic acid sequence encoding any one of the polypeptides of the invention.

In one embodiment, the nucleic acid is an RNA molecule. In one embodiment, the RNA molecule is transcribed from a DNA sequence described herein. In some embodiments, the RNA molecule is encoded by one of the inventive sequences. In another embodiment, the nucleotide sequence comprises an RNA sequence transcribed from a DNA sequence encoding any one of the polypeptide sequences of the invention, or a variant thereof or a fragment thereof. Accordingly, in one embodiment, the invention provides an RNA molecule encoding one or more of inventive envelopes. The RNA may be plus-stranded. Accordingly, in some embodiments, the RNA molecule can be translated by cells without needing any intervening replication steps such as reverse transcription.

In some embodiments, a RNA molecule of the invention may have a 5′ cap (e.g. but not limited to a 7-methylguanosine, 7mG(5′)ppp(5′)NlmpNp). This cap can enhance in vivo translation of the RNA. The 5′ nucleotide of a RNA molecule useful with the invention may have a 5′ triphosphate group. In a capped RNA this may be linked to a 7-methylguanosine via a 5′-to-5′ bridge. A RNA molecule may have a 3′ poly-A tail. It may also include a poly-A polymerase recognition sequence (e.g. AAUAAA) near its 3′ end. In some embodiments, a RNA molecule useful with the invention may be single-stranded. In some embodiments, a RNA molecule useful with the invention may comprise synthetic RNA.

The recombinant nucleic acid sequence can be an optimized nucleic acid sequence. Such optimization can increase or alter the immunogenicity of the envelope. Optimization can also improve transcription and/or translation. Optimization can include one or more of the following: low GC content leader sequence to increase transcription; mRNA stability and codon optimization; addition of a kozak sequence (e.g., GCC ACC) for increased translation; addition of an immunoglobulin (Ig) leader sequence encoding a signal peptide; and eliminating to the extent possible cis-acting sequence motifs (i.e., internal TATA boxes).

In certain aspects the invention contemplates using immunogenic compositions wherein immunogens are delivered as recombinant proteins. Various methods for production and purification of recombinant proteins, including trimers such as but not limited to SOSIP based trimers, suitable for use in immunization are known in the art. In certain embodiments recombinant proteins are produced in CHO cells.

It is readily understood that the envelope glycoproteins referenced in various examples and figures comprise a signal/leader sequence. It is well known in the art that HIV-1 envelope glycoprotein is a secretory protein with a signal or leader peptide sequence that is removed during processing and recombinant expression (without removal of the signal peptide, the protein is not secreted). See for example Li et al. Control of expression, glycosylation, and secretion of HIV-1 gp120 by homologous and heterologous signal sequences. Virology 204(1):266-78 (1994) (“Li et al. 1994”), at first paragraph, and Li et al. Effects of inefficient cleavage of the signal sequence of HIV-1 gp120 on its association with calnexin, folding, and intracellular transport. PNAS 93:9606-9611 (1996) (“Li et al. 1996”), at 9609. Any suitable signal sequence could be used. In some embodiments the leader sequence is the endogenous leader sequence. Most of the gp120 and gp160 amino acid sequences include the endogenous leader sequence. In other non-limiting examples, the leader sequence is human Tissue Plasminogen Activator (TPA) sequence, human CD5 leader sequence (e.g. MPMGSLQPLATLYLLGMLVASVLA (SEQ ID NO:9)). Most of the chimeric designs include CD5 leader sequence. A skilled artisan appreciates that when used as immunogens, and for example when recombinantly produced, the amino acid sequences of these proteins do not comprise the leader peptide sequences.

The immunogenic envelopes can also be administered as a protein prime and/or boost alone or in combination with a variety of nucleic acid envelope primes (e.g., HIV-1 Envs delivered as DNA expressed in viral or bacterial vectors).

Dosing of proteins and nucleic acids can be readily determined by a skilled artisan. A single dose of nucleic acid can range from a few nanograms (ng) to a few micrograms (m) or milligram (mg) of a single immunogenic nucleic acid. Recombinant protein dose can range from a few micrograms (μg) to a few hundred micrograms, or milligrams (mg) of a single immunogenic polypeptide.

Administration: The compositions can be formulated with appropriate carriers using known techniques to yield compositions suitable for various routes of administration. In certain embodiments the compositions are delivered via intramascular (IM), via subcutaneous, via intravenous, via nasal, via mucosal routes, or any other suitable route of immunization.

The compositions can be formulated with appropriate carriers and adjuvants using techniques to yield compositions suitable for immunization. The compositions can include an adjuvant, such as, for example but not limited to 3M052, alum, poly IC, MF-59 or other squalene-based adjuvant, ASOIB, or other liposomal based adjuvant suitable for protein or nucleic acid immunization. In certain embodiments, the adjuvant is GSK ASO IE adjuvant containing MPL and QS21. This adjuvant has been shown by GSK to be as potent as the similar adjuvant AS01B but to be less reactogenic using HBsAg as vaccine antigen (Leroux-Roels et al., IABS Conference, April 2013). In certain embodiments, TLR agonists are used as adjuvants. In other embodiment, adjuvants which break immune tolerance are included in the immunogenic compositions.

In certain embodiments, the compositions and methods comprise any suitable agent or immune modulation which could modulate mechanisms of host immune tolerance and release of the induced antibodies. In non-limiting embodiments modulation includes PD-1 blockade; T regulatory cell depletion; CD40L hyperstimulation; soluble antigen administration, wherein the soluble antigen is designed such that the soluble agent eliminates B cells targeting dominant epitopes, or a combination thereof. In certain embodiments, an immunomodulatory agent is administered in at time and in an amount sufficient for transient modulation of the subject's immune response so as to induce an immune response which comprises broad neutralizing antibodies against HIV-1 envelope. Non-limiting examples of such agents is any one of the agents described herein: e.g. chloroquine (CQ), PTP1B Inhibitor—CAS 765317-72-4—Calbiochem or MSI 1436 clodronate or any other bisphosphonate; a Foxo1 inhibitor, e.g. 344355 Foxo1 Inhibitor, AS1842856—Calbiochem; Gleevac, anti-CD25 antibody, anti-CCR4 Ab, an agent which binds to a B cell receptor for a dominant HIV-1 envelope epitope, or any combination thereof. In non-limiting embodiments, the modulation includes administering an anti-CTLA4 antibody, OX-40 agonists, or a combination thereof. Non-limiting examples are of CTLA-1 antibody are ipilimumab and tremelimumab. In certain embodiments, the methods comprise administering a second immunomodulatory agent, wherein the second and first immunomodulatory agents are different.

Multimeric Envelopes

Presentation of antigens as particulates reduces the B cell receptor affinity necessary for signal transduction and expansion (see Baptista et al. EMBO J. 2000 Feb. 15; 19(4): 513-520). Displaying multiple copies of the antigen on a particle provides an avidity effect that can overcome the low affinity between the antigen and B cell receptor. The initial B cell receptor specific for pathogens can be low affinity, which precludes vaccines from being able to stimulate and expand B cells of interest. In particular, very few naïve B cells from which HIV-1 broadly neutralizing antibodies arise can bind to soluble HIV-1 Envelope. Provided are envelopes, including but not limited to trimers as particulate, high-density array on liposomes or other particles, for example but not limited to nanoparticles. See e.g. He et al. Nature Communications 7, Article number: 12041 (2016), doi:10.1038/ncomms12041; Bamrungsap et al. Nanomedicine, 2012, 7 (8), 1253-1271.

For development as a vaccine immunogen, we have also created multimeric nanoparticles that comprise and/or display HIV envelope protein or fragments on their surface.

The nanoparticle immunogens are composed of various forms of HIV-envelope protein, e.g. without limitation envelope trimer, and self-assembling protein, e.g. without limitation ferritin protein. Any suitable ferritin could be used in the immunogens of the invention. In non-limiting embodiments, the ferritin is derived from Helicobacter pylori. In non-limiting embodiments, the ferritin is insect ferritin. In non-limiting embodiments, each nanoparticle displays 24 copies of the envelope protein on its surface.

Presenting multiple copies of antigens to B cells has been a longstanding approach to improving B cell receptor recognition and antigen uptake (See Batista et al. EMBO J. 2000 Feb. 15; 19(4): 513-520). The improved recognition of antigen is due to the avid interaction of multiple antigens with multiple B cell receptors on a single B cells, which results in clustering of B cells and stronger cell signaling. Furthermore, multimeric presentation improves antigen binding to mannose binding lectin which promotes antigen trafficking to B cell follicles. Self-assembling complexes comprising multiple copies of an antigen are one strategy of immunogen design approach for arraying multiple copies of an antigen for recognition by the B cell receptors on B cells (Kanekiyo, M., Wei, C. J., Yassine, H. M., McTamney, P. M., Boyington, J. C., Whittle, J. R., Rao, S. S., Kong, W. P., Wang, L., and Nabel, G. J. (2013). Self-assembling influenza nanoparticle vaccines elicit broadly neutralizing H1N1 antibodies. Nature 499, 102-106; Ueda, G., Antanasijevic, A., Fallas, J. A., Sheffler, W., Copps, J., Ellis, D., Hutchinson, G. B., Moyer, A., Yasmeen, A., Tsybovsky, Y., et al. (2020). Tailored design of protein nanoparticle scaffolds for multivalent presentation of viral glycoprotein antigens. Elife).

In some instances, the gene of an antigen can be fused via a linker/spacer to a gene of a protein which could self-assemble. Upon translation, a fusion protein is made that can self-assemble into a multimeric complex—also referred to as a nanoparticle displaying multiple copies of the antigen. In other instances, the protein antigen could be conjugated to the self-assembling protein via an enzymatic reaction, thereby forming a nanoparticle displaying multiple copies of the antigen. Non-limiting embodiments of enzymatic conjugation include without limitation sortase mediated conjugation. In some embodiments, linkers for use in any of the designs of the invention could be 2-50 amino acids long, e.g. 2, 3, 4, 5, 6, 7, 8, 9, 10, 11, 12, 13, 14, 15, 16, 17, 18, 19, 20, 25, 30, 35, 40, 45, or 50 amino acids long. In certain embodiments, these linkers comprise glycine and serine amino acid in any suitable combination, and/or repeating units of combinations of glycine, serine and/or alanine.

Ferritin is a well-known protein that self-assembles into a hollow particle composed of repeating subunits. In some species ferritin nanoparticles are composed of 24 copies of a single subunit, whereas in other species it is composed of 12 copies each of two subunits.

Non-limiting embodiments of sortase linkers could be used so long as their position allows multimerization of the envelopes. In a non-limiting embodiment, a C-terminal tag is LPXTG (SEQ ID NO: 10), where X signifies any amino acid but most commonly Ala, Ser, Glu, or a N-terminal pentaglycine repeat tag is added to the envelope trimer gene. In a non-limiting embodiment, a C-terminal tag is LPXTGG (SEQ ID NO: 196), where X signifies any amino acid but most commonly Ala, Ser, Glu.

To improve the interaction between the naïve B cell receptor and immunogens, envelope designed can be created to wherein the envelope is presented on particles, e.g. but not limited to nanoparticle. In some embodiments, the HIV-1 Envelope trimer could be fused to ferritin. Ferritin protein self assembles into a small nanoparticle with three fold axis of symmetry. At these axes the envelope protein is fused. Therefore, the assembly of the three-fold axis also clusters three HIV-1 envelope protomers together to form an envelope trimer. Each ferritin particle has 8 axes which equates to 8 trimers being displayed per particle. See e.g. Sliepen et al. Retrovirology 2015 12:82, DOI: 10.1186/s12977-015-0210-4.

Any suitable ferritin sequence could be used. In non-limiting embodiments, ferritin sequences are disclosed in WO/2018/005558.

Ferritin nanoparticle linkers: The ability to form HIV-1 envelope ferritin nanoparticles relies self-assembly of 24 ferritin subunits into a single ferritin nanoparticle. The addition of a ferritin subunit to the c-terminus of HIV-1 envelope may interfere with the ability of the ferritin subunit to fold properly and or associate with other ferritin subunits. When expressed alone ferritin readily forms 24-subunit nanoparticles, however appending it to envelope only yields nanoparticles for certain envelopes. Since the ferritin nanoparticle forms in the absence of envelope, the envelope could be sterically hindering the association of ferritin subunits. Thus, ferritin can be designed with elongated glycine-serine linkers to further distance the envelope from the ferritin subunit. To make sure that the glycine linker is attached to ferritin at the correct position, constructs can be created that attach at second amino acid position or the fifth amino acid position. The first four n-terminal amino acids of natural Helicobacter pylori ferritin are not needed for nanoparticle formation but may be critical for proper folding and oligomerization when appended to envelope. Thus, constructs can be designed with and without the leucine, serine, and lysine amino acids following the glycine-serine linker. The goal will be to find a linker length that is suitable for formation of envelope nanoparticles when ferritin is appended to most envelopes. Any suitable linker between the envelope and ferritin could be uses, so long as the fusion protein is expressed and the trimer is formed.

Another approach to multimerize expression constructs uses Staphylococcus sortase A transpeptidase ligation to conjugate inventive envelope trimers to cholesterol. The trimers can then be embedded into liposomes via the conjugated cholesterol. To conjugate the trimer to cholesterol either a C-terminal LPXTG (SEQ ID NO:10) tag, where X signifies any amino acid but most commonly Ala, Ser, Glu, or a N-terminal pentaglycine repeat tag is added to the envelope trimer gene. Cholesterol is also synthesized with these two tags. Sortase A is then used to covalently bond the tagged envelope to the cholesterol. The sortase A-tagged trimer protein can also be used to conjugate the trimer to other peptides, proteins, or fluorescent labels. In non-limiting embodiments, the sortase A tagged trimers are conjugated to ferritin to form nanoparticles. See FIG. 18.

The invention provides design of envelopes and trimer designs wherein the envelope comprises a linker which permits addition of a lipid, such as but not limited to cholesterol, via a sortase A reaction. See e.g. Tsukiji, S. and Nagamune, T. (2009), Sortase-Mediated Ligation: A Gift from Gram-Positive Bacteria to Protein Engineering. ChemBioChem, 10: 787-798. doi:10.1002/cbic.200800724; Proft, T. Sortase-mediated protein ligation: an emerging biotechnology tool for protein modification and immobilisation. Biotechnol Lett (2010) 32: 1. doi:10.1007/s10529-009-0116-0; Lena Schmohl, Dirk Schwarzer, Sortase-mediated ligations for the site-specific modification of proteins, Current Opinion in Chemical Biology, Volume 22, October 2014, Pages 122-128, ISSN 1367-5931, dx.doi.org/10.1016/j.cbpa.2014.09.020; Tabata et al. Anticancer Res. 2015 August; 35(8):4411-7; Pritz et al. J. Org. Chem. 2007, 72, 3909-3912.

The lipid modified envelopes and trimers could be formulated as liposomes. Any suitable liposome composition is contemplated.

Non-limiting embodiments of envelope designs for use in sortase A reaction are shown in FIG. 24 B-D of WO2017/151801, incorporated by reference in its entirety.

Additional sortase linkers could be used so long as their position allows multimerization of the envelopes. In a non-limiting embodiment, a C-terminal tag is LPXTG (SEQ ID NO:10), where X signifies any amino acid but most commonly Ala, Ser, Glu, or a N-terminal pentaglycine repeat tag is added to the envelope trimer gene. In a non-limiting embodiment, a C-terminal tag is LPXTGG (SEQ ID NO: 196), where X signifies any amino acid but most commonly Ala, Ser, Glu.

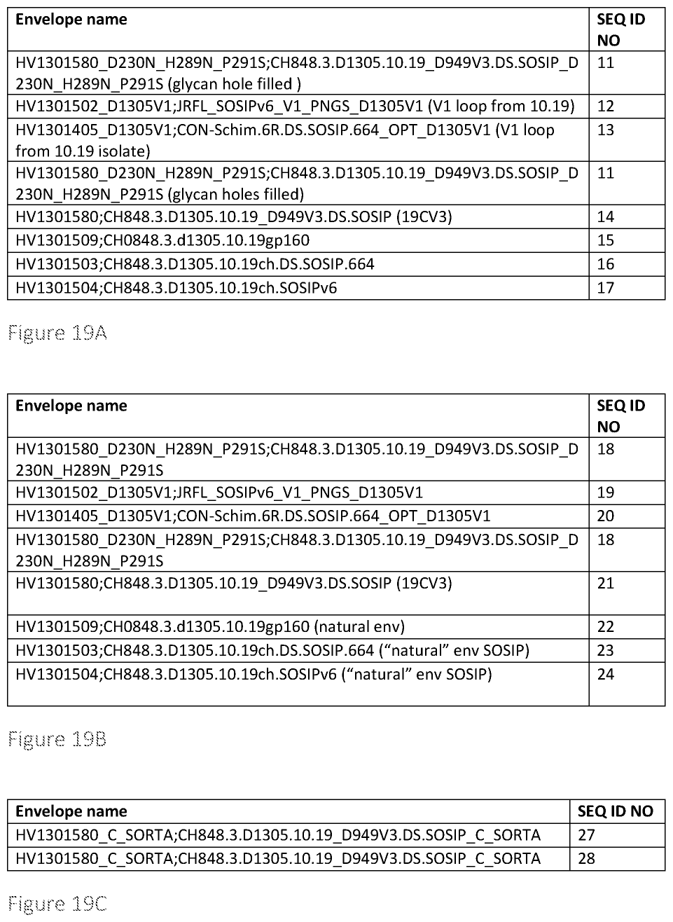

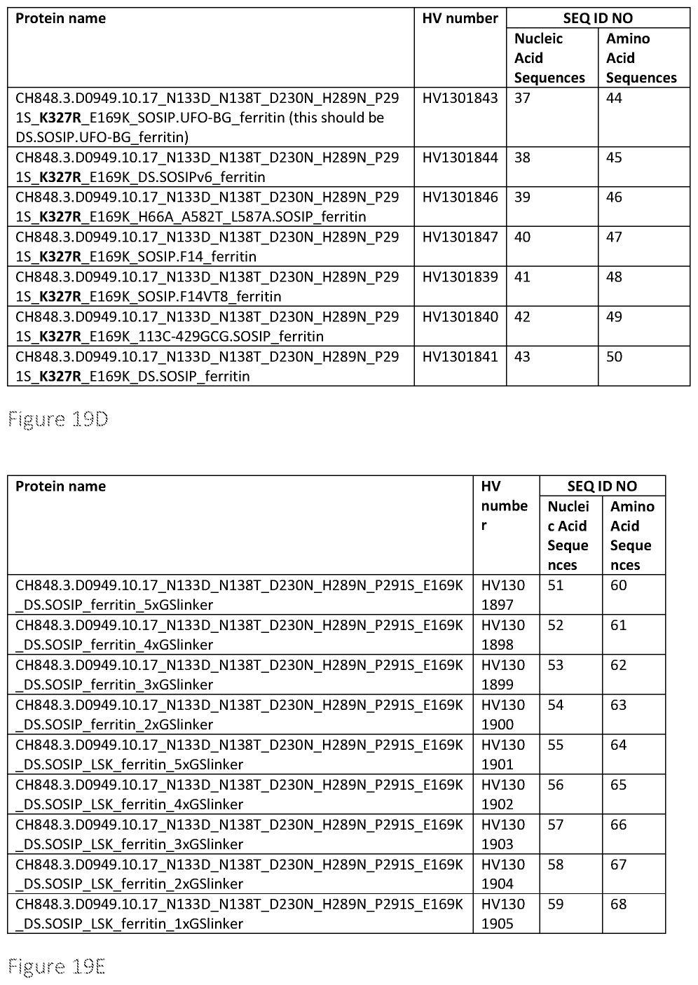

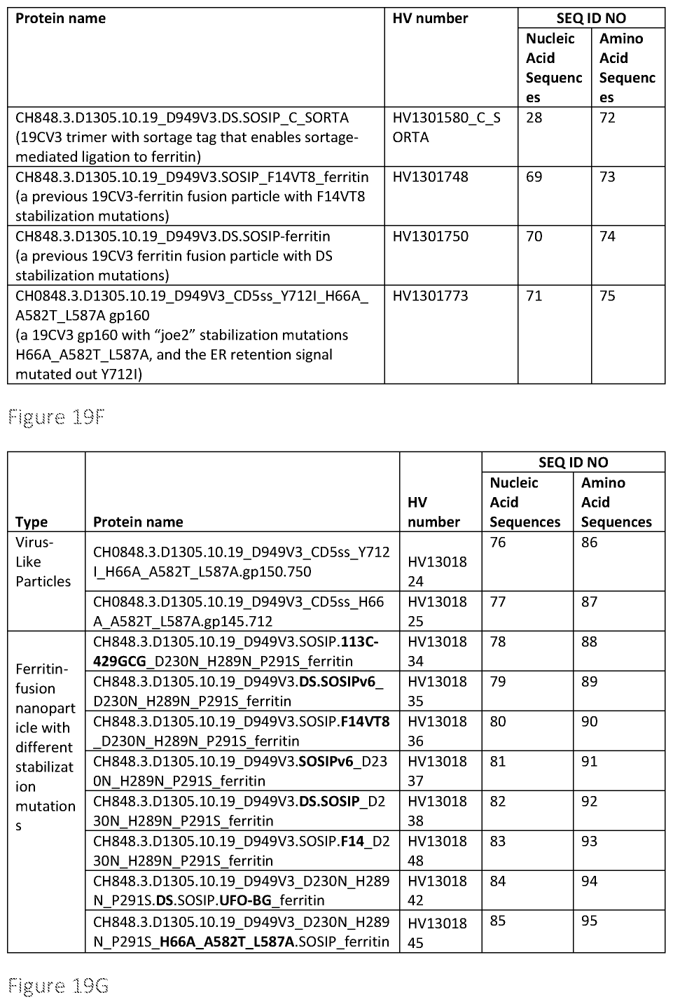

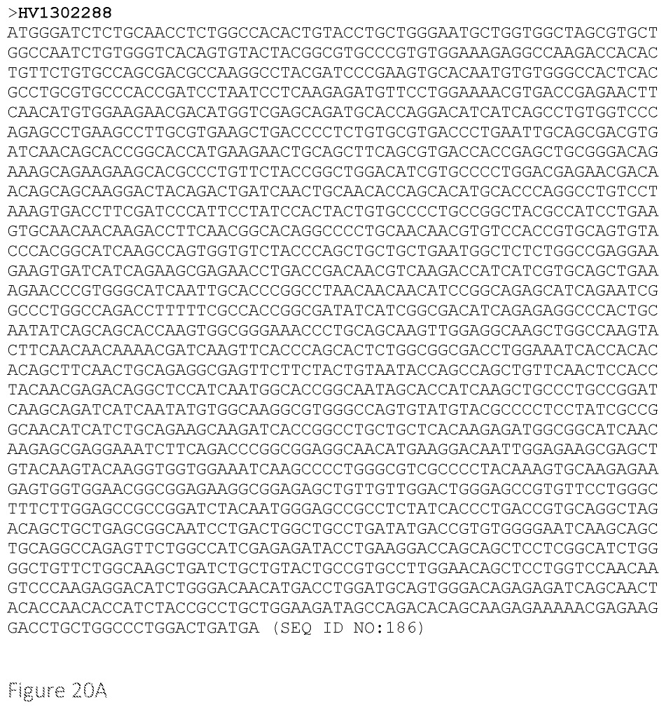

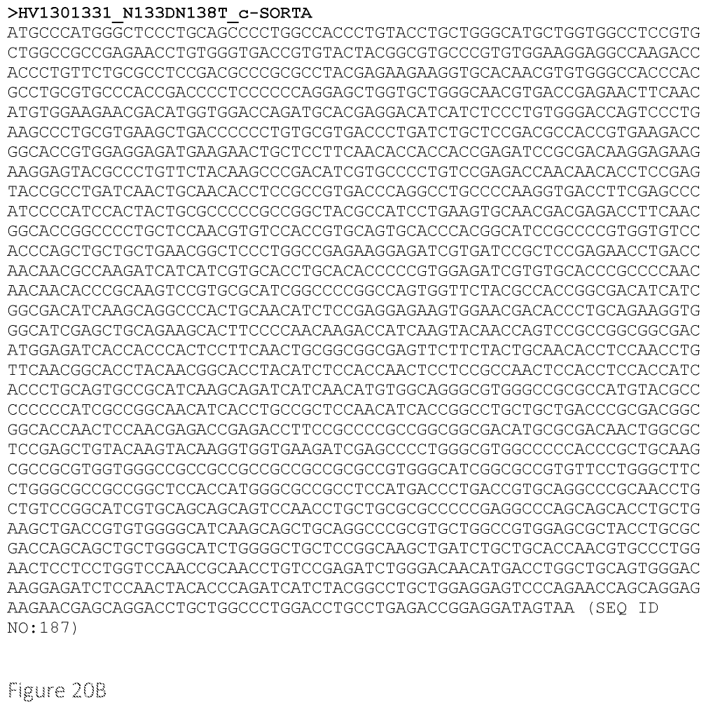

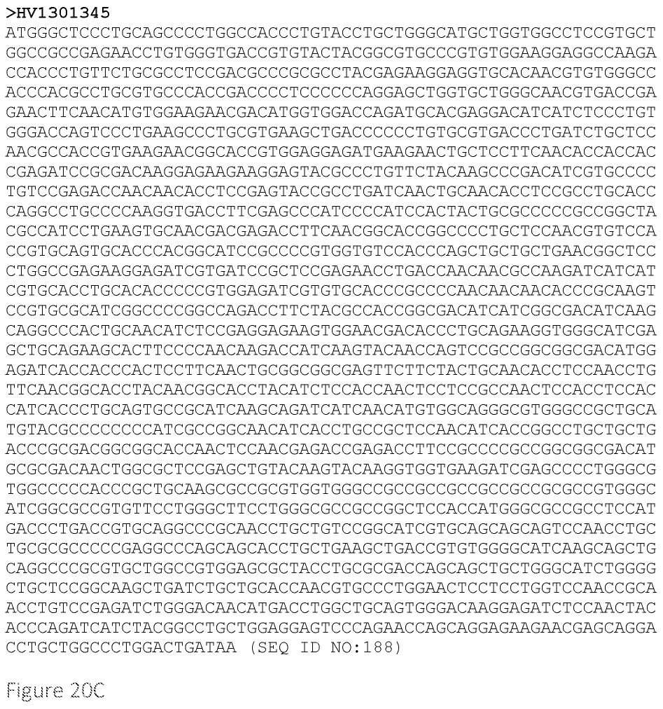

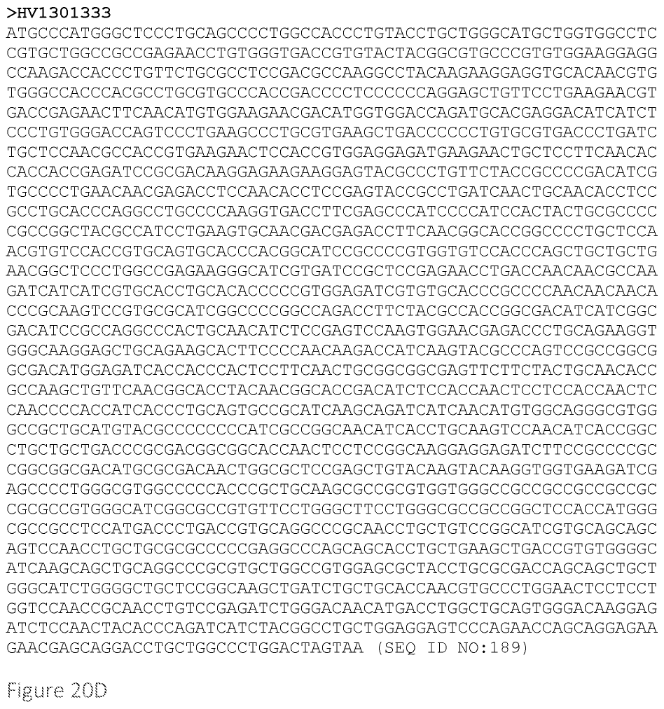

Table 1 shows a summary of sequences described herein.

| | Amino acid (aa) or nucleic | | |
|---|---|---|---|
| Name | acid (nt) | Design | Figure/Note |
| HV1301580_D230N_H289N_P291S; | Nt | | FIG. 19A |
| CH848.3.D1305.10.19_D949V3.DS.SOSIP_D230N_H289N_P291S | aa | | FIG. 19B |
| (glycan hole filled) | | | |
| >HV1301502_D1305V1; | Nt | | FIG. 19A |
| JRFL_SOSIPv6_V1_PNGS_D1305V1 | aa | | FIG. 19B |
| (V1 loop from 10.19) | | | |
| >HV1301405_D1305V1; CON- | Nt | | FIG. 19A |
| Schim.6R.DS.SOSIP.664_OPT_D1305V1 | aa | | FIG. 19B |
| (V1 loop from 10.19 isolate) | | | |
| >HV1301580_D230N_H289N_P291S; | Nt | | FIG. 19A |
| CH848.3.D1305.10.19_D949V3.DS.SOSIP_D230N_H289N_P291S | aa | | FIG. 19B |
| (glycan holes filled) | | | |
| >HV1301580; | Nt | 19CV3 | FIG. 19A |
| CH848.3.D1305.10.19_D949V3.DS.SOSIP | aa | | FIG. 19B |
| (19CV3) | | | |
| >HV1301509; | Nt | | FIG. 19A |
| CH0848.3.d1305.10.19gp160 | aa | | FIG. 19B |
| >HV1301503; | Nt | | FIG. 19A |
| CH848.3.D1305.10.19ch.DS.SOSIP.664 | aa | | FIG. 19B |
| >HV1301504; | Nt | | FIG. 19A |
| CH848.3.D1305.10.19ch.SOSIPv6 | aa | | FIG. 19B |
| >HV1301580_C_SORTA; | Aa | | FIG. 19C |
| CH848.3.D1305.10.19_D949V3.DS.SOSIP_C_SORTA | nt | | FIG. 19C |

Table 2 shows a summary of modifications to envelopes described herein

| | | | | V3 glycosylation | UCA and other |
|---|---|---|---|---|---|
| Row | Envelope | Figure/SEQ ID No | V1 region | sites | Ab binding |
| 1 | 10.17 | See PCT | 17aa | N301 and N332 | |
| | | Publication | | | |
| | | WO2018161049 | | | |
| 2 | 10.17 DT | See PCT | 17aa N133D | N301 and N332 | DH270UCA |
| | | Publication | N138T | | |
| | | WO2018161049 | effectively lacks | | |
| | | | glycosylation | | |
| | | | sites | | |
| 3 | 10.19 | FIG. 19 | 17aa V1 region | No glycosylation | CH01 UCA |
| | | | lacks N133 and | sites at N295, | |
| | | | N138 | N301, N332 | |
| | | | glycosylation | | |
| | | | sites | | |
| 4 | 10.19 plus V3 | FIG. 19 | 17aa V1 region | Add V3 regions | CH01 UCA |
| | loop of 10.17 | | lacks N133 and | from 10.17 has | DH270UCA |
| | (19CV3) | | N138 | five aa difference | VRC26 UCA |
| | | | glycosylation | from 10.19 | |
| | | | sites | | |
| 5 | 10.19 env | FIG. 19 | | At least changes | |
| | based with | | | #2, 4, 5, and/or | |
| | fewer than | | | “GDIR” (SEQ ID | |
| | five aa | | | NO: 1) sequence | |
| | changes | | | | |
| | compared to | | | | |
| | 19CV3; | | | | |
| | “GDIR/K” | | | | |
| 6 | Ferritin | FIG. 19 | | | |
| | Linker | | | | |
| 7 | E169K | FIG. 19 | | | |
| 8 | Glycan hole | FIG. 19 | | | |
| | filled | | | | |

DH270 light chain binds to N301 glycan. In some embodiments, a N301 gly site is used (e.g. change #2 in row 5 of Table 2, supra).

DH270 heavy chain binds to N332 glycan. In some embodiments, a N332 gly site is used (e.g. changes #4 and #5 in row 5 of Table 2, supra).

V3 glycan Abs bind GDIR (SEQ ID NO:1). In some embodiments, a change #3 to “GDIR” (SEQ ID NO:1) is needed (e.g. “GDIR” sequence (SEQ ID NO:1) in row 5 of Table 2, supra).

GDIR/K motif: V3-glycan broadly neutralizing antibodies typically contact the c-terminal end of the third variable region on HIV-1 envelope. There are four amino acids, Gly324, Asp325, Ile326, and Arg327, bound by V3-glycan neutralizing antibodies. While Arg327 is highly conserved among HIV-1 isolates, Lys327 also occurs at this site. The CH848.3.D0949.10.17 isolate naturally encodes the less common Lys327. In contrast to CH848.3.D0949.10.17 with the Lys327, the precursor antibody of the DH270 V3-glycan broadly neutralizing antibody lineage barely binds to CH848.3.D0949.10.17 encoding Arg327. Thus, Arg327 is critical for the precursor to bind and the lineage of neutralizing antibodies to begin maturation. However, somatically mutating antibodies on the path to developing neutralization breadth bind better to Env encoding Arg327. See FIG. 14. Thus, Env must encode Lys327 to initiate DH270 lineage development. However, to best interact with affinity maturing DH270 lineage members the Env should encode Arg327. Thus, a plausible vaccine regimen to initiate and select for developing bnAbs would include a priming immunogen encoding, Lys327 and a boosting immunogen encoding Arg327. The Arg327 boosting immunogen would optimally target the affinity maturing DH270 lineage members, while not optimally binding the DH270 antibodies that lack affinity maturation. Non-limiting embodiments of vaccination regimens could include: priming with CH848.3.D0949.10.17 based envelope design also with Lys327, followed by administering of CH848.3.D0949.10.17 based envelope design with Arg327. Non-limiting embodiments of vaccination regimens could include: priming with 19CV3 based envelope design also with Lys327, followed by administering of CH848.3.D0949.10.17 based envelope design with Arg327.

E169K modification: One approach to designing a protective HIV-1 vaccine is to elicit broadly neutralizing antibodies (bnAbs). However, bnAbs against two or more epitopes will likely need to be elicited to prevent HIV-1 escape. Thus, optimal HIV-1 immunogens should be antigenic for multiple bnAbs in order to elicit bnAbs to more than one epitope. The CH848.D949.10.17 HIV-1 isolate was antigenic for V3-glycan antibodies but lacked binding to V1V2-glycan antibodies. Not all viruses from the CH848 individual lacked binding to V1V2-glycan antibodies. For example, the CH848.D1305.10.19 isolate bound well to V1V2-glycan antibody PGT145. We compared the sequence of CH848.D949.10.17 and CH848.D1305.10.19 in the region that is contacted by V1V2-glycan antibodies in crystal structures (McLellan J S, Pancera M, Carrico C, Gorman J, Julien J P, Khayat R, et al. Structure of HIV-1 gp120 V1/V2 domain with broadly neutralizing antibody PG9. Nature. 2011; 480(7377):336-43). Interestingly, the CH848.D949.10.17 and CH848.D1305.10.19 differed in sequence at a known contact site for V1V2-glycan antibodies—position 169 (Doria-Rose N A, Georgiev I, O'Dell S, Chuang G Y, Staupe R P, McLellan J S, et al. A short segment of the HIV-1 gp120 V1/V2 region is a major determinant of resistance to V1/V2 neutralizing antibodies. J Virol. 2012; 86(15):8319-23). It has been previously shown that mutation of lysine at position 169 eliminates binding to V1V2-glycan antibody PG9 (Doria-Rose N A, Georgiev I, O'Dell S, Chuang G Y, Staupe R P, McLellan J S, et al. A short segment of the HIV-1 gp120 V1/V2 region is a major determinant of resistance to V1/V2 neutralizing antibodies. J Virol. 2012; 86(15):8319-23). CH848.D1305.10.19 sequence encoded a lysine at position 169 whereas CH848.D949.10.17 sequence encoded a glutamate. Thus, we changed the glutamate (E) to lysine (K) at position 169 of CH848.D949.10.17. This single change in CH848.D949.10.17 enabled V1V2-glycan antibody binding to the envelope. Thus, the E169K adds the V1V2-glycan epitope to the other bnAb epitopes present on CH848.D949.10.17-based envelopes. Overall, the result of the E169K is a CH848.D949.10.17 envelope capable of eliciting more different types of bnAbs.

The invention contemplates any other design, e.g. stabilized trimer, of the sequences described here in. For non-limiting embodiments of additional stabilized trimers see WO2014/042669 (DU4061), WO2017/151801 (DU4716), WO2017/152146 (DU4918), WO2018/161049 (DU4918), and WO/2020/072169 (F14 and/or VT8 designs) all of which are incorporated by reference in their entirety.

In certain embodiments the invention provides an envelope comprising 17aa V1 region without N133 and N138 glycosylation, and N301 and N332 glycosylation sites, and further comprising “GDIR” motif (SEQ ID NO: 1), wherein the envelope binds to UCAs of V1V2 Abs and V3 Abs.

TABLE 3
Summary of envelope designs.

| | | | | Figure with a |
|---|---|---|---|---|
| | | | | non-limiting |
| HV | Envelope to be used as protein and/or | Linker | Ferritin for | sequence |
| name | mRNA | sequence | multimerization | embodiment |
| HV1302144 | 10.17 DTe protein trimer | no | No | FIG. 1B, 1F |
| | CH848.3.D0949.10.17_N133D_N138T_D230N_H289N_P291S_E169K_{—} | | | |
| | DS.chSOSIP | | | |
| HV1302144_cSortA | 10.17 DTe protein sortaNP | For Sortase | Any | |
| | CH848.3.D0949.10.17_N133D_N138T_D230N_H289N_P291S_E169K_{—} | conjugation | Ferritin is | |
| | DS.chSOSIP_cSortA | | added via | |
| | | | Sortase | |
| | | | conjugation | |
| | | | at C- | |
| | | | terminus | |
| HV1301925 | 10.17 DTe protein NP | SGG | could be | FIG. 1A, 1E |
| | CH848.3.D0949.10.17_N133D_N138T_D230N_H289N_P291S_E169K_{—} | | any | |
| | DS.chSOSIP_VRCferritin | | suitable | |
| | | | ferritin | |
| | 10.17WTe designs | | | |
| HV1302145 | 10.17WTe protein trimer | no | | FIG. 1C, 1G |
| | CH848.3.D0949.10.17_D230N_H289N_P291S_E169K_DS.chSOSIP | | | |
| HV1302145_cSortA | CH848.3.D0949.10.17_D230N_H289N_P291S_E169K_DS.chSOSIP_cSortA | For Sortase | Any | |
| | | conjugation | Ferritin is | |
| | | | added via | |
| | | | Sortase | |
| | | | conjugation | |
| | | | at C- | |
| | | | terminus | |
| HV1302146 | CH848.3.D0949.10.17_D230N_H289N_P291S_E169K_DS.chSOSIP_{—} | SGG | could be | FIG. 1D, 1H |
| | VRCferritin | | any | |
| | | | suitable | |
| | | | ferritin | |

TABLE 4
Summary of selection of immunogens for induction of neutralizing antibodies.

| | | NEW selection | One embodiment | Figures | Envelope protomer |
|---|---|---|---|---|---|
| prime | | CH848.0949.10.17DT | See Table 3, see | | Various protomer |
| | | Or | FIG. 1I | | designs, including |
| | | CH848.0949.10.17DTe | | | without limitation various |
| | | | | | stabilized designs. |
| boost | x | CH848.0949.10.17 | See Table 3, see | | Various protomer |
| | | or | FIG. 1I | | designs, including |
| | | CH848.0949.10.17WTe | | | without limitation various |
| | | | | | stabilized designs. |
| boost | | CH848.0808.15.15 | CH0848.3.d0808.15.15.MB | See Fig. | CH0848.3.d0808.15.15.MB |
| | | | 6R.DS.SOSIP.664_CD5ss | 2C | 6R.DS.SOSIP.664 |
| | | | | | With various signal |
| | | | | | peptides |
| | | | | | Various protomer |
| | | | | | designs, including |
| | | | | | without limitation various |
| | | | | | stabilized designs. |
| boost | x | CH848.0358.80.06 | | See Fig. | |
| | | | | 3U | |
| boost | x | CH848.1432.5.41 | | See Fig. | |
| | | | | 3U | |
| boost | | CH848.1621.4.44 | CH0848.3.d1621.4.44 | See Fig. | CH0848.3.d1621.4.44 |
| | | | 6R.DS.SOSIP.664_CD5ss | 2C | 6R.DS.SOSIP.664 |
| | | | | | With various signal |
| | | | | | peptides |
| | | | | | Various protomer |
| | | | | | designs, including |
| | | | | | without limitation various |
| | | | | | stabilized designs |
| boost | | CH848.1305.10.35 | CH0848.3.d1305.10.35 | See Fig. | CH0848.3.d1305.10.35 |
| | | | 6R.DS.SOSIP.664_CD5ss | 2C | 6R.DS.SOSIP.664 |
| | | | | | With various signal |
| | | | | | peptides |
| | | | | | Various protomer |
| | | | | | designs, including |
| | | | | | without limitation various |
| | | | | | stabilized designs |
| boost | | P0402.c2.11 (G) | | See Fig. | Various protomer |
| | | | | 2C | designs, including |
| | | | | | without limitation various |
| | | | | | stabilized designs |
| boost | | ZM246F (C) | | See Fig. | Various protomer |
| | | | | 2C | designs, including |
| | | | | | without limitation various |
| | | | | | stabilized designs |

(x) indicates non-limiting embodiments of boost envelopes described in Example 1.

Throughout the specification, the name CH848.d0949.10.17 DT is interchangeably used as CH848.d0949.10.17.N133D.N138T. Throughout the specification, the name CH848.d0949.10.17 is interchangeably used as CH848.d0949.10.17WT. In certain embodiments, CH848.d0949.10.17DT envelope comprises additional modifications D230N.H289N.P291S.E169K and is referred to as CH848.d0949.10.17 DTe. In certain embodiments, CH848.d0949.10.17 envelope comprises additional modifications D230N.H289N.P291S.E169K and is referred to as CH848.d0949.10.17WTe.

Any suitable signal peptide could be used. In designs comprising ferritin for multimerization, any suitable linker could be used between the envelope sequence and a ferritin sequence. When recombinantly produced, proteins do not comprise a signal peptide which is cleaved during recombinant protein production.

### EXAMPLES

### Example 1

This example provides analyses and selection of a new set of immunogens for induction of HIV-1 neutralizing antibodies.

Vaccines that can induce anti-HIV-1 broadly neutralizing antibodies (bNAbs) remain highly sought after as they will induce broad protective responses that will prevent infection by the globally diverse HIV-1 strains. We and others have shown that such bNAbs arise in HIV-1 infected individuals through multiple rounds of virus escape followed by antibody hypermutation to learn recognition of these escaped viruses (e.g. Bonsignori et al. Sci Transl Med 2017 Mar. 15; 9(381):eaai7514. doi: 10.1126/scitranslmed.aai7514, PMID: 28298420). In this application we outline the selection of a set of sequential immunogens that are designed to mimic this process through vaccination.

In Bonsignori et al., PMID: 28298420 we reported the development of DH270.6, a bNAb targeting V3 glycan epitope, in the HIV-1 infected individual CH848. This antibody lineage was traced to identify intermediates along the evolutionary trajectory, and several viruses from CH848 were tested for neutralization against these intermediate and mature bNAbs.

In this work, we first identified signatures, defined as amino acids, glycan sites and hypervariable loop characteristics that are statistically associated with sensitivity or resistance to DH270 lineage Abs (Bricault et al. Cell Host Microbe. 2019 Jan. 9; 25(1):59-72.e8. doi: 10.1016/j.chom.2018.12.001. PMID: 30629920). These signatures were calculated for both CH848 viruses as well as global HIV-1 viruses.

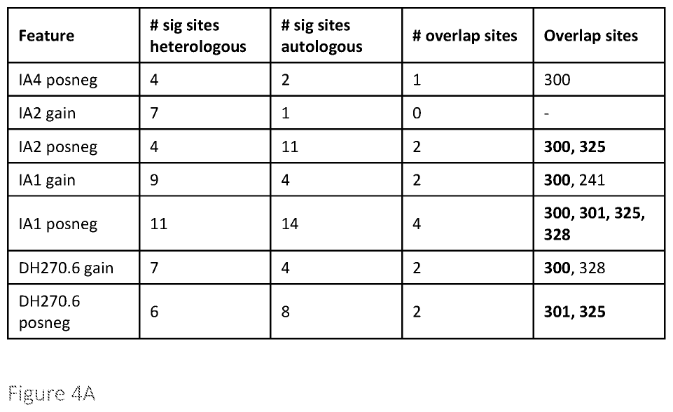

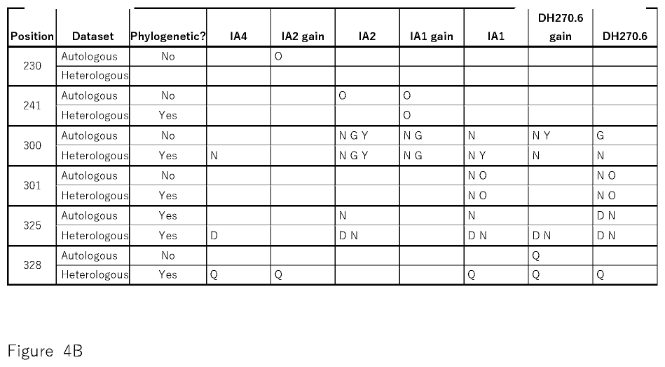

We found that six positions (HXB2: 230, 241, 300, 301, 325 & 328) and hypervariable V1 loop lengths were statistically significant signatures that were overlapping between the two analyses. We hypothesize that these common signature sites of viral sensitivity/escape against DH270 antibodies in the CH848 patient viruses as well as global HIV-1 viruses are key positions at which CH848 viral evolution “teaches” the DH270 lineage to recognize heterologous HIV-1 diversity. See FIGS. 4-8.

In FIG. 5 and throughout FIGS. 4-8 and Example 1, where reference is made to education sites or signature sites or signature amino acids, amino acids are represented adjacent to each other. These adjacent amino acids do not represent a continuous peptide, but rather amino acids at the signature sites positions: 230, 241, 300, 301, 325 and 328.

Analyses of the longitudinal evolution of CH848 envelopes showed that TF variants associated with resistance to early Abs at sites 230 & 300. These evolve to sensitive variants 779-1119 days post infection, with a timeline for IA4˜779-892 days post infection. Relapse to NxST-230 is never at high frequency, but at low frequency at day 948. IA2 likely arises. Day 1304-1634 onwards we start seeing escape at 241, 300, 301, 325, 328 towards breadth gain variants. IA1 likely arises. Day 1650 onwards Y-300 becomes dominant, and full resistance associated H-301 and N-325 become more prevalent. DH270.6 likely arises.

Analyses of bnAb education sites show the following. IA4-sensitive: CH848.d0949.10.17 matches all sensitive variants. Only autologous signatures are G-336 and L-337 sensitive. 10.17 does not match these (E-336 K-337). IA2 breadth gain: 230 NxST associated with IA2 breadth gain (only autologous). E-325 is quite rare in heterologous viruses. IA1 breadth gain: Breadth gain variants: K-241, G/Y-300 & K-328. N-325 recognized at very low frequency. DH270.6/.4 gain: Y-300 & K-328 are better recognized. N-325 is still rarely recognized. Resistance to all DH270 lineage: Main routes of escape loss of NxST301 & N325 (heterologous only NxST-332 viruses considered).

Position N300 has structural relevance. IA4 and IA2 both require Asn-300 but later lineage members can tolerate G/Y. N-300 forms a polar contact with N-302. N300G or N300Y could disrupt this, and potentially change orientation of 301 glycan. 301 glycan is important: critical and improbable mutations might interact S27Y, Y93F (light) and G110Y (heavy). NxST 442 is quite rare in M-group.

Structural relevance of positions Q328 and D325 have. For Q328K: A4 & IA2 prefer Q-328. IA1 begins to see K-328 and DH270.6 can tolerate Q & K equally. Q-328 forms a polar contact with T-148. So Q-328 be involved in sequestering V1 loop away from V3. IA1 onwards longer V1 loops are tolerated. For D325N: D-325 strictly required IA4 & IA2. N-325 is rarely tolerated by IA1 & DH270.6, and enriched in viruses resistant to all DH270 Abs. D325 inserts between CDRH2 & CDRH3, could have made contacts, but does not. Closest Ab aa is D107. R-57 is not close (˜9 Å).

IA1 breadth gain loop signatures: Hyp V1+V2 length significantly associated in both heterologous and autologous datasets (p=0.0002-0.0033). IA4 & IA2 in autologous dataset recognize very small loops, but IA1 onwards can tolerate longer loops.

For CH848 viruses, dramatic length change for V1, but very little for V2.

Previous immunogen design including envelopes 10.17DT, d0948.10.17, d0835.10.31, d0357.80.06, d1431.5.41, d0525.25.02 missed patterns Y-300, N-325 & K-241. See FIG. 6.

FIG. 7 shows one embodiments of a new immunogen design based on neutralization profiles and coverage of key breadth-gain and resistance signatures. Three immunogens from previous design were retained: d949.10.17, d358.80.06 and d1432.5.41. Three new immunogens are added: D808.15.15 introduces NxST-230; d1621.4.44 introduces Y-300; d1305.10.35 introduces N-325. No suitable Envs with K-241 found—either too short or too long V1. It is rare in M-group, so this position was ignored. Table 4 and FIGS. 1-3 show non-limiting examples of these envelopes.

We used this hypothesis to guide the selection of seven CH848 Envs that not only show appropriate neutralization profiles against DH270 Abs but also expose critical amino acids at the above 6 positions and appropriate hypervariable V1 loops in a sequential manner that upon vaccination are designed to initiate and mature antibody responses similar to DH270. These Envs are: 1) CH848.d0949.10.17 DT, 2) CH848.d0949.10.17, 3) CH848.d0808.15.15, 4) CH848.d0358.80.06, 5) CH848.d1432.5.41, 6) CH848.d1621.4.44 and 7) CH848.d1305.10.35.

We have also calculated signatures that are associated with restricting the breadth of the broadest DH270 Ab (DH270.6), and have chosen two natural Envs (P0402.c2.11 and ZM246F) that expose such resistant signatures at sites 325 and 301, respectively, with the rationale that boosting with these two Env immunogens could induce DH270-like Abs that can show higher breadth than DH270.6. See FIG. 8.

In the Figures for this example and in this example, the term “global” panel is the same as “heterologous” panel. The heterologous viruses refers to a standard panel of 208 global circulating Envs made as pseudotyped viruses that is used for testing neutralization breadth and potency of antibodies. This was the same panel that was used in Bonsignori et al. Sci Transl Med 2017 Mar. 15; 9(381):eaai7514. doi: 10.1126/scitranslmed.aai7514, PMID: 28298420. The “autologous” panel is the 90 pseudovirus panel made using strategically chosen longitudinal CH848 Envs from Bonsignori et al. (PMID: 28298420).

In the figures in this example, the phylogenetic correction refers to a particular strategy that accounts for potential biases arising from Glade effects in signature calculations, as described in previous publications (Bhattacharya et al. Science. 2007 Mar. 16; 315(5818):1583-6. doi: 10.1126/science.1131528. PMID: 17363674; Bricault et al. Cell Host Microbe. 2019 Jan. 9; 25(1):59-72.e8. doi: 10.1016/j.chom.2018.12.001. PMID: 30629920).

The symbol “O” is a short-hand of indicating an Asparagine in a potential N-linked glycosylation site motif (Asn-x-Ser/Thr, where x is any amino acid other than Pro). “N” refers to Asn not in such motifs.

The envelope selection is based on comparison of heterologous and autologous signatures to find overlap. This analysis identified 6 sites that have similar patterns across DH270 Abs between heterologous and autologous datasets—bNAb education. Based on these analyses, we designed a set of immunogens.

Below tables shows DH270 UCA knock-in mice for immunization studies. The immunogens could have any suitable envelope design, e.g, without limitation envelope trimer, envelope comprised in a nanoparticle, so forth. Immunogens could be delivered in any suitable form, including without limitation proteins, nucleic acids, e.g. mRNA, formulation in any adjuvant. In non-limiting embodiments, the envelopes are be delivered as nanoparticles, trimers, and/or nucleic acids.

TABLE 5
DH270 UCA knock-in mice for immunization studies. The immunogens could have any suitable
envelope design, e.g, without limitation envelope trimer, envelope comprised in a nanoparticle,
so forth. Immunogens could be delivered in any suitable form, including without limitation
proteins, nucleic acids, e.g. mRNA, formulation in any adjuvant.

| Study | 1 | 2 | 3 | 4 | 5 | 6 | 7 | 8 |
|---|---|---|---|---|---|---|---|---|
| New #1 | 10.17DT | 10.17DT | 10.17DT | 10.17WT | 15.15 | 80.06 | 5.41 | 5.41 |
| | NP | NP | NP | trimer | trimer | trimer | 4.44 | 4.44 |
| | | | | | | | trimers | 10.35 |
| | | | | | | | | P0402 |
| | | | | | | | | ZM246 |
| New #2 | 10.17DT | 10.17DT | 10.17DT | 10.17WT | 10.17WT | 10.17WT | 10.17WT | 5.41 |
| | NP | NP | NP | 15.15 | 15.15 | 15.15 | 15.15 | 4.44 |
| | | | | 80.06 | 80.06 | 80.06 | 80.06 | P0402 |
| | | | | 5.41 | 5.41 | 5.41 | 5.41 | 10.35 |
| | | | | 4.44 | 4.44 | 4.44 | 4.44 | ZM246 |
| New #3 | 10.17DT | 10.17DT | 10.17DT | 10.17WT | 15.15 | 80.06 | 5.41 | 5.41 |
| | NP | NP | 10.17WT | 15.15 | 80.06 | 5.41 | 4.44 | 4.44 |
| | | | | | | 4.44 | P0402 | P0402 |
| | | | | | | | | 10.35 |
| | | | | | | | | ZM246 |

TABLE 6
One embodiment of a mouse study. In this study 5.41 and 4.44 are grouped together as
they show similar neutralization profiles (only sensitive to IA1 & DH270.6). Envelope
10.35 (N-325) and the two heterologous viruses come at the last step. Studies can compare
Ab responses before and after this to study the impact of these immunogens designed to go
beyond DH270.6. Envelopes 5.41 & 4.44 also included so that the more resistant viruses
(10.35 + 2 het) could drive off-target responses. An alternative is where the 8^{th}
immunization is a repeat of 7^{th}, and 9^{th} is 10.35 & 2 heterologous envelopes.

| Study | 1 | 2 | 3 | 4 | 5 | 6 | 7 | 8 |
|---|---|---|---|---|---|---|---|---|
| New #1 | 10.17DT | 10.17DT | 10.17DT | 10.17WT | 15.15 | 80.06 | 5.41 | 5.41 |
| | NP | NP | NP | trimer | trimer | trimer | 4.44 | 4.44 |
| | | | | | | | trimers | 10.35 |
| | | | | | | | | P0402 |
| | | | | | | | | ZM246 |

TABLE 7
One embodiment of an animal study. In this study, immunogens from 10.17 WT (for IA4 targeting)
to 4.44 (IA1 & DH270.6 targeting) co-delivered 4 times to test if boosting with mixture can
lead to better Ab responses (compared with New #1). Final step is for going beyond DH270.6.

| Study | 1 | 2 | 3 | 4 | 5 | 6 | 7 | 8 |
|---|---|---|---|---|---|---|---|---|
| New #2 | 10.17DT | 10.17DT | 10.17DT | 10.17WT | 10.17WT | 10.17WT | 10.17WT | 5.41 |
| | NP | NP | NP | 15.15 | 15.15 | 15.15 | 15.15 | 4.44 |
| | | | | 80.06 | 80.06 | 80.06 | 80.06 | P0402 |
| | | | | 5.41 | 5.41 | 5.41 | 5.41 | 10.35 |
| | | | | 4.44 | 4.44 | 4.44 | 4.44 | ZM246 |

TABLE 8
One embodiment of an animal study. In this embodiment each boost designed
to target 2 Ab intermediates in each step. (e.g. 3^{rd} boost for UCA + IA4, 4^{th}
boost for IA4 & IA2, etc.) The 7^{th} boost - P0402 is included before 10.35 (which
is in 8^{th} immunization) because P0402 sensitive to both IA1 & DH270.6,
while 10.35 only for DH270.6 (UG021 completely resistant).

| Study | 1 | 2 | 3 | 4 | 5 | 6 | 7 | 8 |
|---|---|---|---|---|---|---|---|---|
| New #3 | 10.17DT | 10.17DT | 10.17DT | 10.17WT | 15.15 | 80.06 | 5.41 | 5.41 |
| | NP | NP | 10.17WT | 15.15 | 80.06 | 5.41 | 4.44 | 4.44 |
| | | | | | | 4.44 | P0402 | P0402 |
| | | | | | | | | 10.35 |
| | | | | | | | | ZM246 |

TABLE 9
Summary of DH270 UCA knock-in mice immunization studies

| Study | 1 | 2 | 3 | 4 | 5 | 6 | 7 | 8 |
|---|---|---|---|---|---|---|---|---|
| MU445 | 10.17DT | 10.17DT | 10.17DT | 10.17DT | 10.17DT | 10.17DT | | |
| | NP | NP | NP | NP | NP | NP | | |
| MU486 | 10.17DT | 10.17DT | 10.17DT | 10.17WT | 10.31 | 80.06 | 5.41 | 25.02 |
| Group | NP | NP | NP | trimer | trimer | trimer | trimer | trimer |
| 1 | | | | | | | | |
| MU486 | 10.17DT | 10.17DT | 10.17DT | 10.17WT | 10.17WT | 10.17WT | 10.17WT | 10.17WT |
| Group | NP | NP | NP | trimer | trimer | trimer | trimer | trimer |
| 2 | | | | | | | | |
| New | 10.17DT | 10.17DT | 10.17DT | 10.17WT | 15.15 | 80.06 | 5.41 | 5.41 |
| imm #1 | NP | NP | NP | trimer | trimer | trimer | 4.44 | 4.44 |
| | | | | | | | trimers | 10.35 |
| | | | | | | | | P0402 |
| | | | | | | | | ZM246 |
| New | 10.17DT | 10.17DT | 10.17DT | 10.17WT | 10.17WT | 10.17WT | 10.17WT | 5.41 |
| imm #2 | NP | NP | NP | 15.15 | 15.15 | 15.15 | 15.15 | 4.44 |
| | | | | 80.06 | 80.06 | 80.06 | 80.06 | 10.35 |
| | | | | 5.41 | 5.41 | 5.41 | 5.41 | P0402 |
| | | | | 4.44 | 4.44 | 4.44 | 4.44 | ZM246 |
| New | 10.17DT | 10.17DT | 10.17DT | 10.17WT | 15.15 | 80.06 | 5.41 | 5.41 |
| imm #3 | NP | NP | 10.17WT | 15.15 | 80.06 | 5.41 | 4.44 | 4.44 |
| | | | | | | 4.44 | P0402 | 10.35 |
| | | | | | | | | P0402 |
| | | | | | | | | ZM246 |

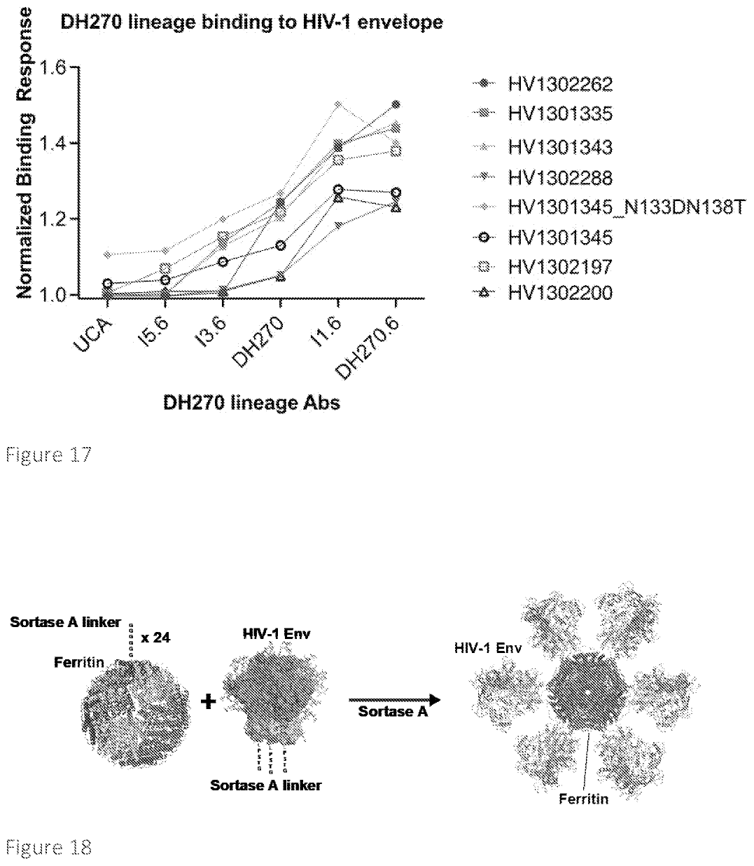

Antigenicity of a selection of these envelopes was tested against various antibodies and the results are shown in FIG. 17. From FIG. 17, the staged reactivity showed that certain DH270 antibodies acquired reactivity with selected envelopes at specific points of affinity maturation. For example, P0402 envelope did not bind to lowly mutated antibodies (Dh270 UCA, 15.6, or 13.6), but the antibody lineage acquired binding once it mutated to the DH270.1 stage of maturation. This differential binding is expected to facilitate selection of antibodies during the affinity maturation process. Administering these envelopes in sequence could select for affinity maturation of DH270-like antibodies into a bnAb.

These immunogens will be tested in mouse studies or any other suitable animal model.

Animal studies analyzing the immunogens from this example will be conducted to evaluate the immune responses induced by this selection of immunogen.

Any suitable adjuvant will be used. The number and time interval between boost can be determined experimentally.

The envelopes described in Table 3 or Table 4, expressed as recombinant proteins or modified mRNA formulated in LNP, are analyzed in animal studies including mouse and NHP animal models. The mouse animal model could be any model, including an animal model comprising a DH270UCA transgene.

### Example 2

This example describes animal studies with HIV-1 envelopes designed to prime and boost V3 glycan antibodies lineages. See FIGS. 9-15.

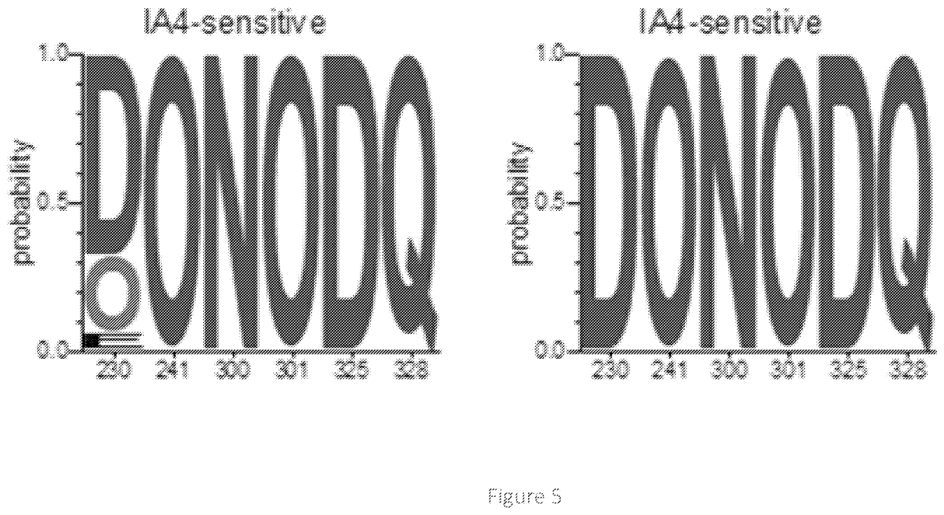

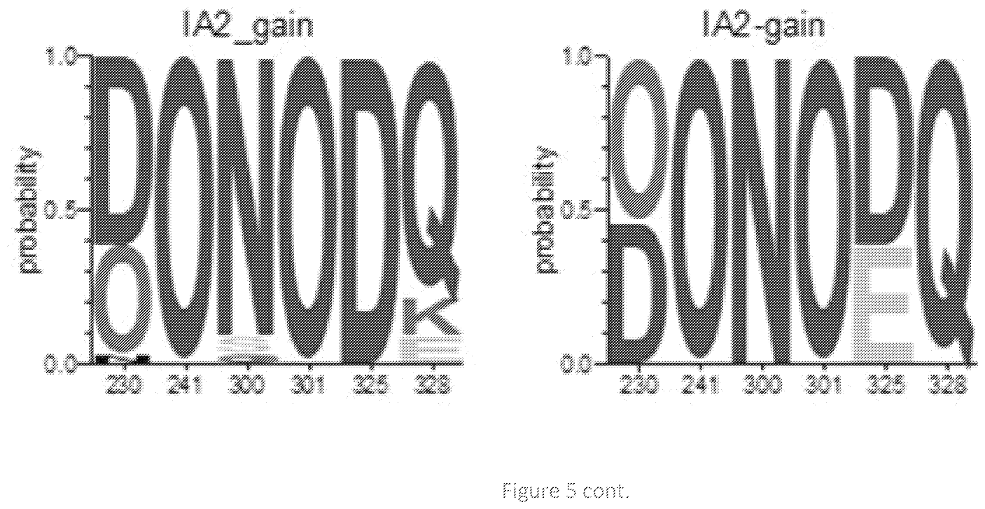

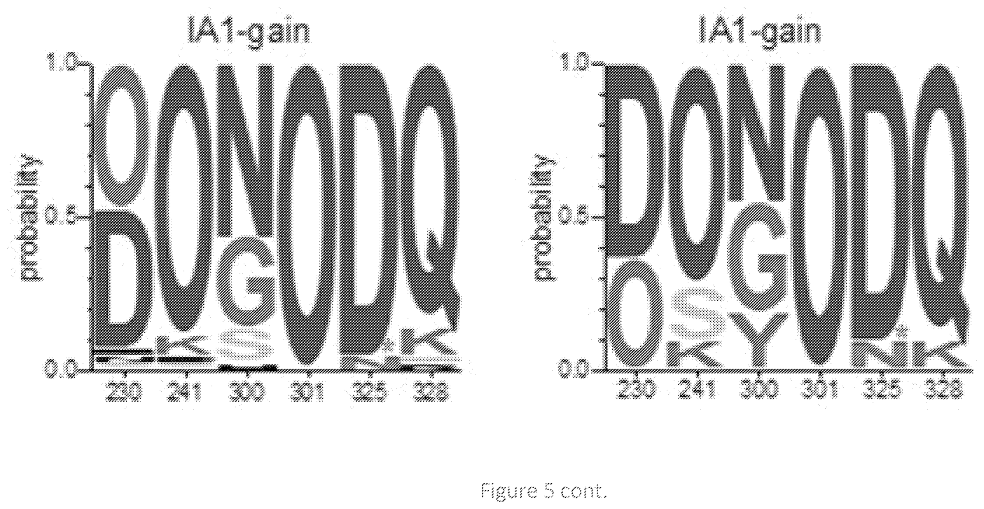

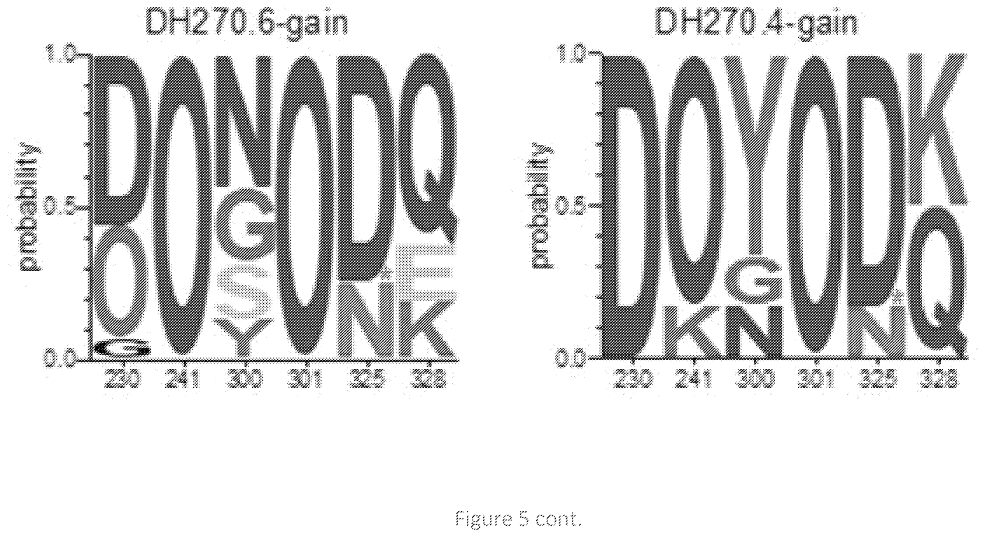

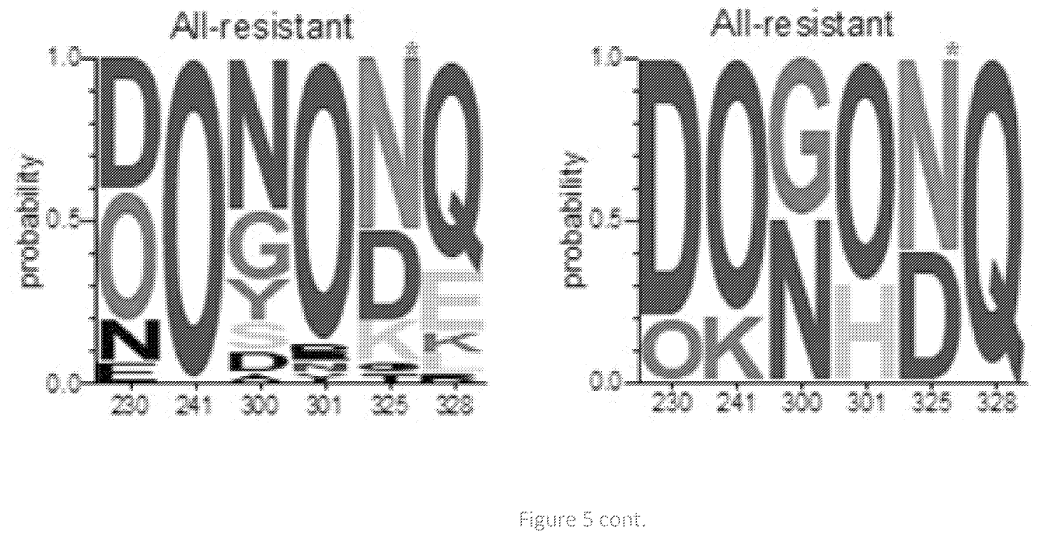

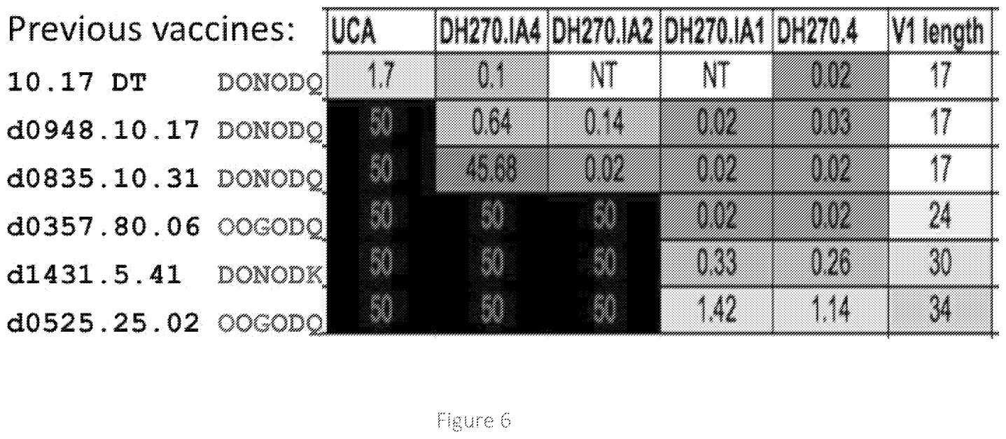

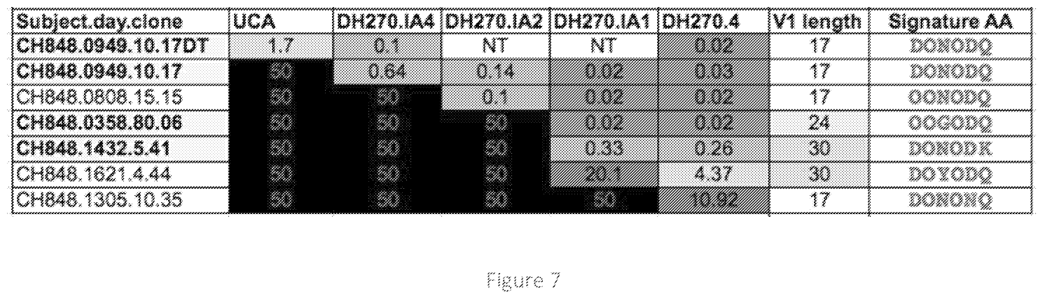

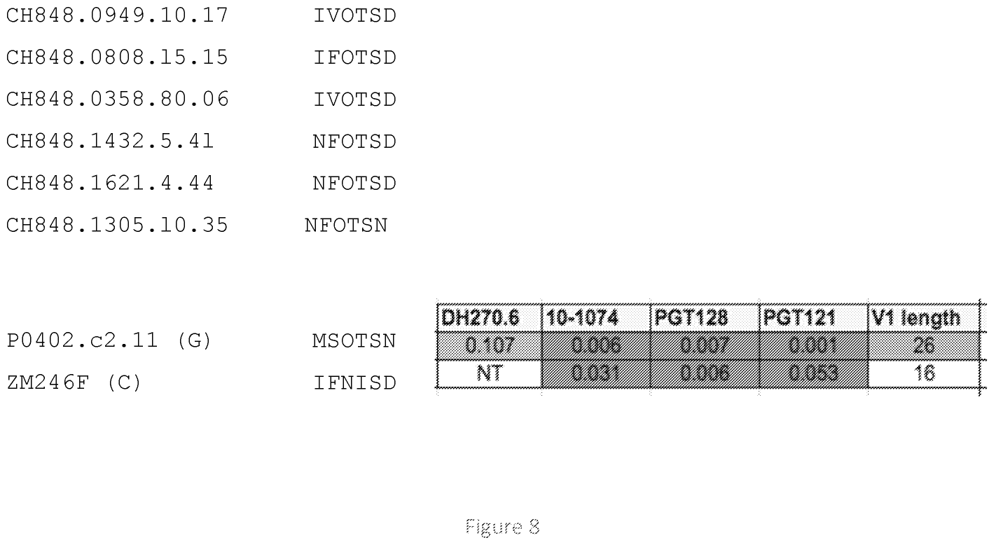

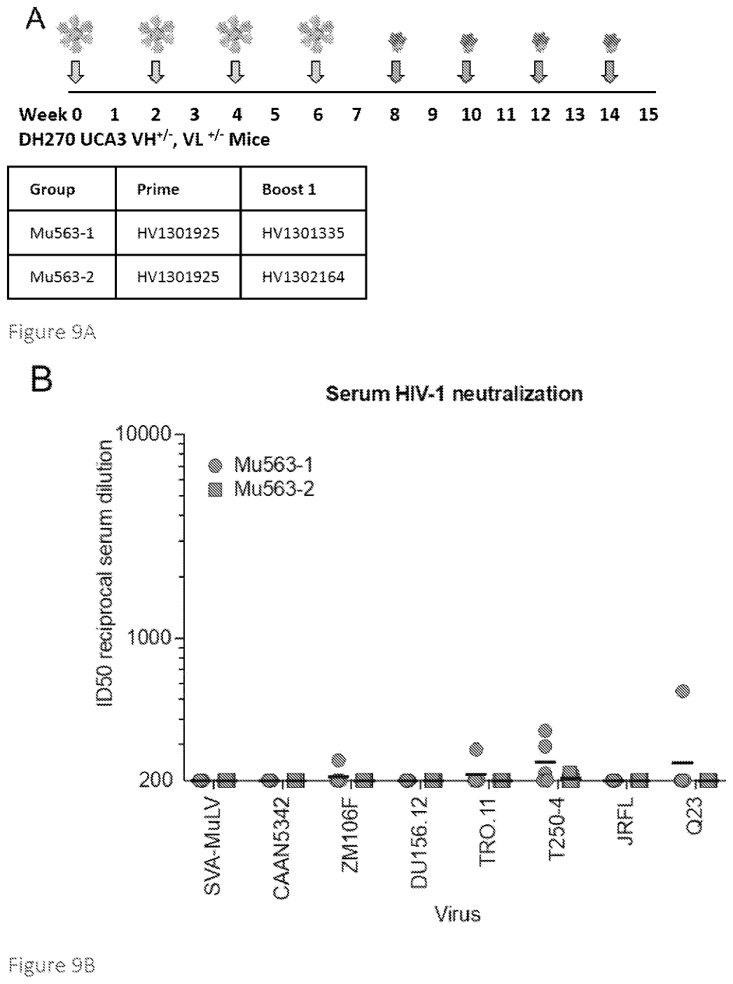

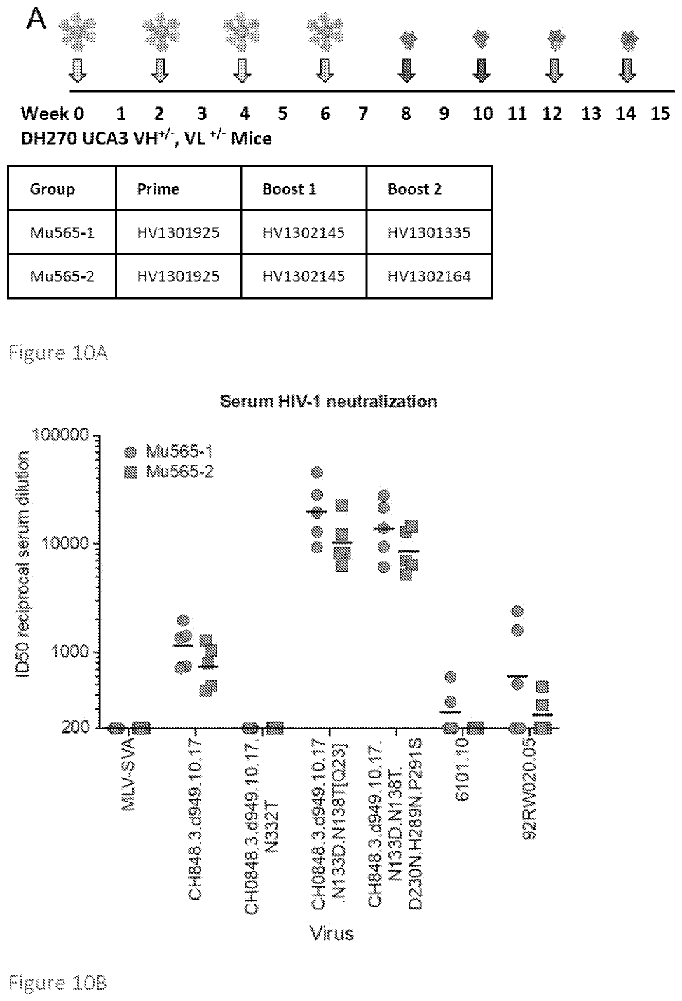

In FIGS. 9 and 10, the Mu563 and Mu565 studies compared the boosting effects of HV1301335 and HV1302164 preceded by the HV1302145 envelope or administered directly after HV1301925 prime. The regimen including HV1301335 was superior to the regimen with HV1302164 in eliciting heterologous neutralizing.

The heterologous neutralization of 92RW020 was enhanced by including the HV1302145 envelope as the second boost (compare Mu563-1 vs Mu565-1).

FIGS. 11A-11C show data for vaccine elicitation of serum neutralization in mouse studies Mu486-2 and Mu486-1. 11A shows DH270 UCA3 VH^{+/-}, VL^{+/-} mouse immunization regimen. Int this figure, Serum neutralization titer against autologous (11B) and heterologous (11C) viruses shown as serum dilution required to inhibit 50% of virus replication one week after the final immunization. N133D N138T matches the priming immunogen for both groups. N332T is a knockout mutation for the antibody of interest DH270. SVA is a negative control unrelated virus. Horizontal line represents group geometric mean. Each symbol represents an individual mouse. This study compared different boosting immunogens. Boosting with CH848.D949.10.17 (Mu486-2) elicited superior serum neutralization titers compared to boosting with a sequence of 5 different envelopes (Mu486-1). This study also investigated vaccine induction of critical mutations for antibody function. Amino acid sequence comparison between vaccine-induced antibodies and DH270 natural lineage antibodies. These antibodies were induced by vaccination and show that vaccination is inducing somatic mutation of DH270 antibodies. Mutations in the VH that are critical to select for are G57R and R98T. Both of these somatic mutations are selected by the vaccine regimen in group 2. In the VL of DH270, S27Y and L48Y are critical for neutralization activity. Both S27Y and L48Y were selected for in the vaccine-elicited antibodies. Prime-boost vaccination is eliciting antibodies with the critical somatic mutations needed for broad neutralization.

FIGS. 12A-12B show vaccine elicitation of serum neutralization in mouse study Mu534-1. DH270 UCA3 VH^{1+/-}, VL^{+/-} mouse immunization regimen is in FIG. 12A. FIG. 12B shows serum neutralization titer against autologous and heterologous viruses shown as serum dilution required to inhibit 50% of virus replication one week after the final immunization. N133D N138T matches the priming immunogen for both groups. N332T is a knockout mutation for the antibody of interest DH270. SVA is a negative control unrelated virus. Horizontal line represents group geometric mean. Each symbol represents an individual mouse. This study is a repeat of Mu486-2. This study showed that CH848.10.17 DT scNP followed by 10.17 trimer induction of heterologous neutralizing antibodies was reproducible. Induction of 92RW020 neutralization is a virus that is sensitive to affinity matured DH270 antibodies but not the UCA. Serum antibodies from the vaccinate mice neutralize 92RW020 indicating the DH270 antibodies have evolved.

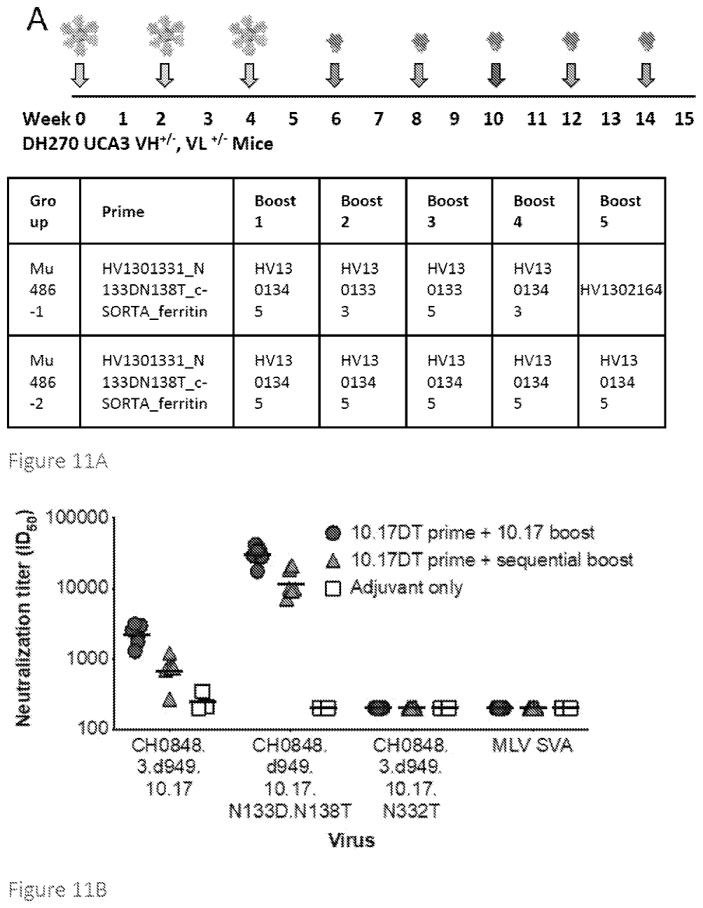

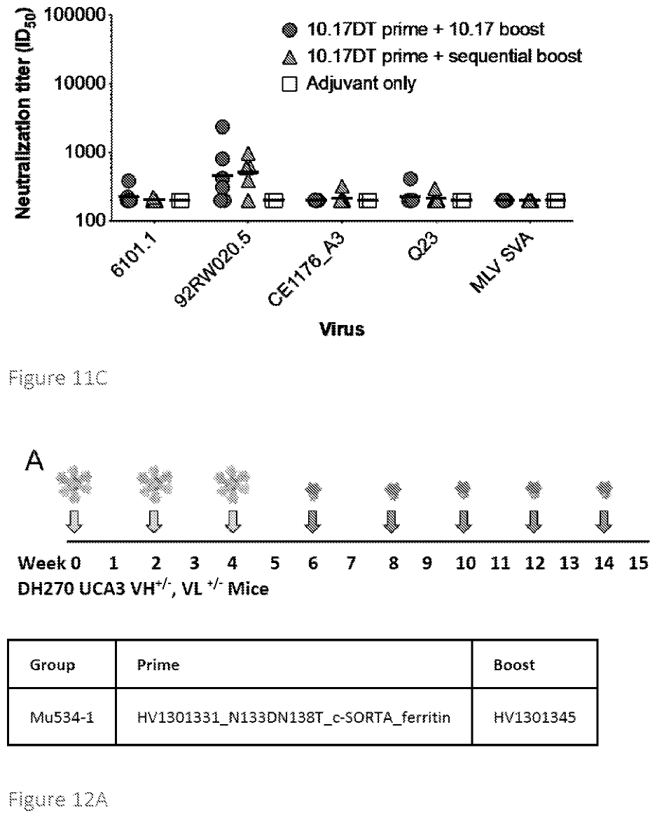

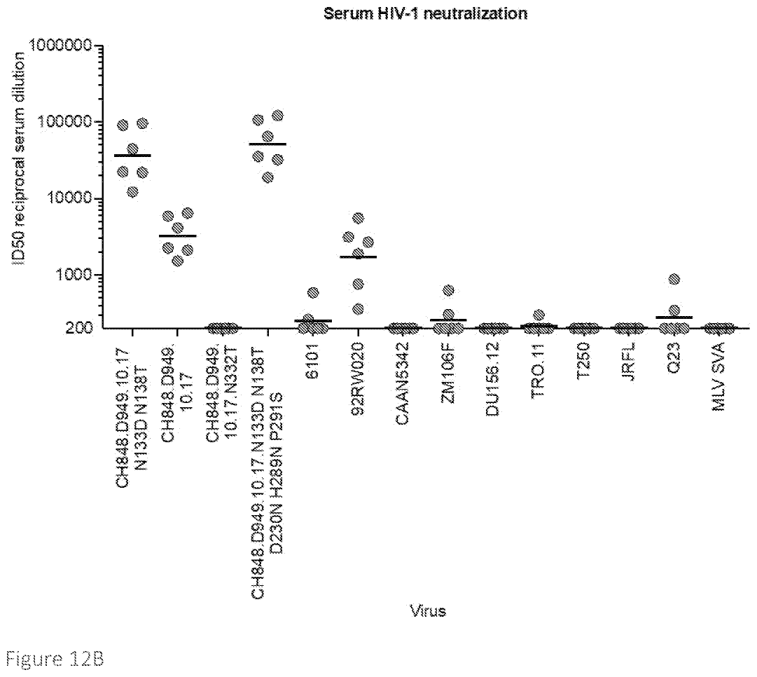

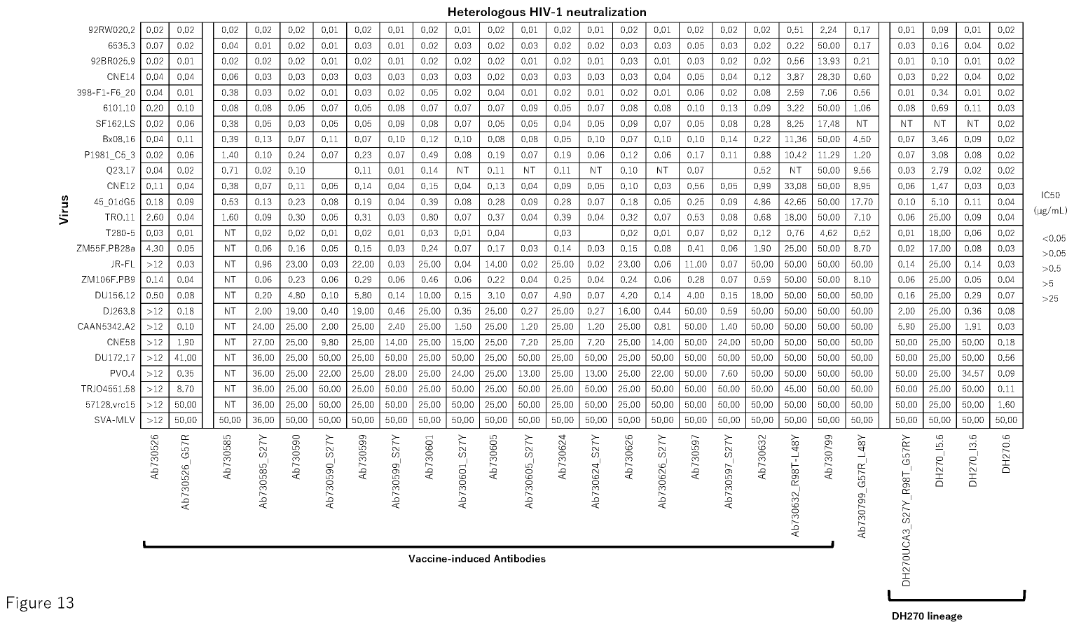

FIG. 13 shows neutralization breadth elicited by prime-boost vaccine regimen. DH270 UCA3 VH^{+/-}, VL^{+/-} mouse immunization regimen as in FIG. 11—mouse studies Mu486-2 and Mu486-1. B. MAb neutralization titer IC50 as mcg per mL required to inhibit 50% of virus replication. Ab730526 was isolated from group 1. The remaining antibodies were isolated from group 2. NT, not tested. These antibodies were induced by vaccination. These vaccine-induced antibodies shown here exhibit broad neutralization. Their neutralization is superior to Dh270.I5.6 which is the first inferred node of the DH270.6 phylogeny. Each antibody has a second version with an additional mutation added (_X), which shows that the artificial addition of DH270 somatic mutations to the antibody further improves neutralization breadth. These artificially-mutated antibodies indicate the mutations that are needed to be selected by the next set of vaccine immunogens.

FIGS. 14A-D show Next generation sequencing of heavy chain variable regions shows vaccine selection of critical functional improbable mutations needed for DH270 antibody affinity maturation. In FIG. 14A, the table describes each DH270 UCA3 VH^{+/-}, VL^{+/-} mouse immunization regimen. FIGS. 14B-D, respectively, describe the frequency of the observed somatic mutation. Adjuvant only groups are shown as Adj only GLA-SE for protein immunization and Adj only LNP for mRNA. Group median is shown by horizontal bars. Mu546 and Mu547 studies delivered the envelopes as mRNAs. All other groups used proteins.

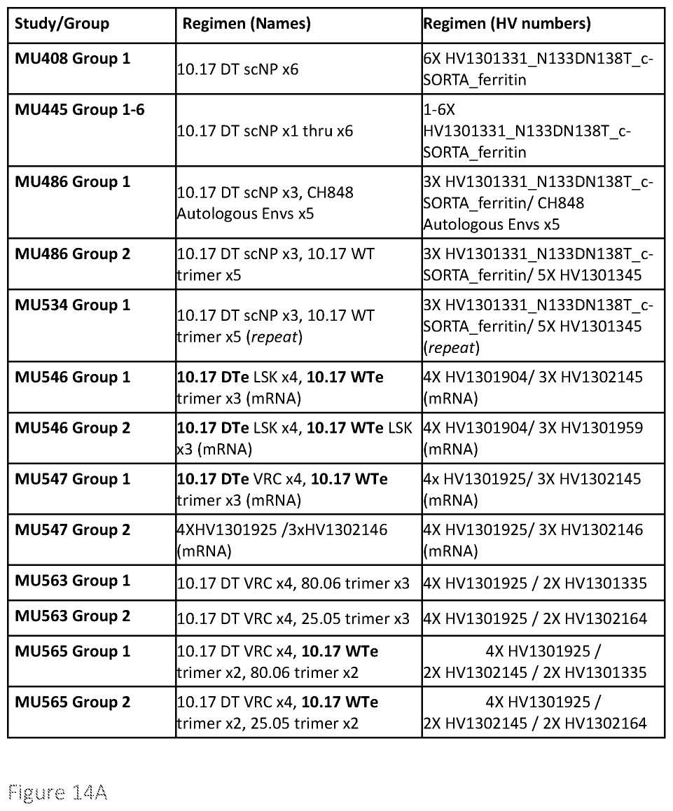

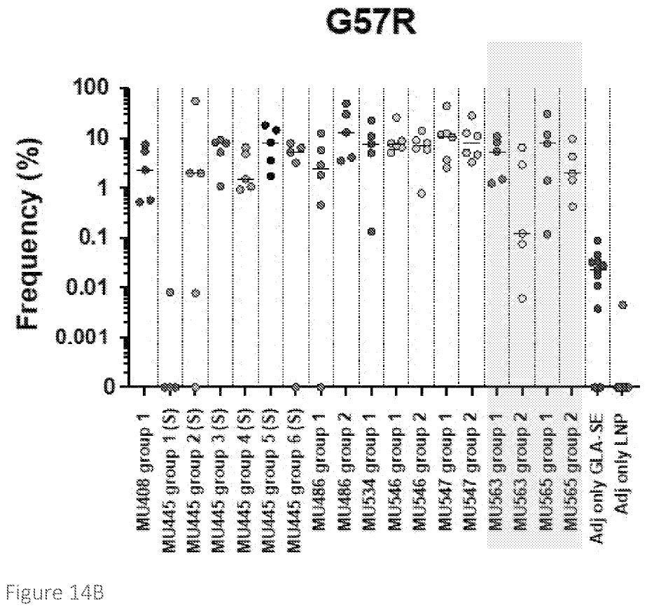

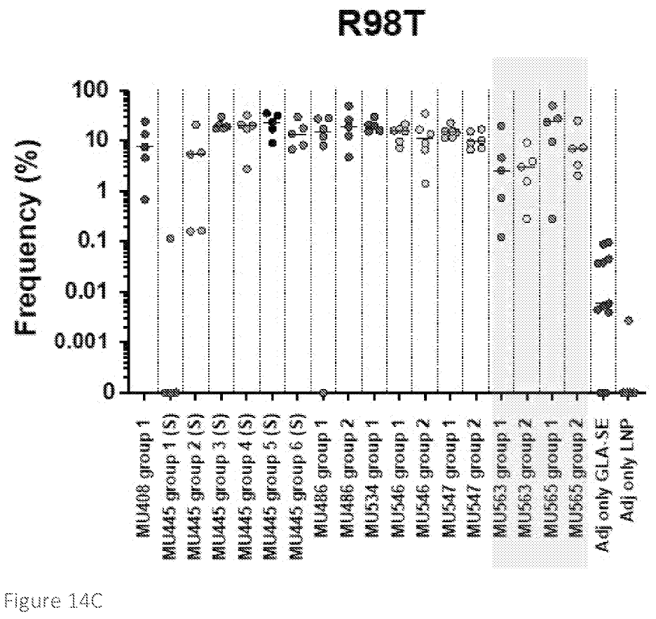

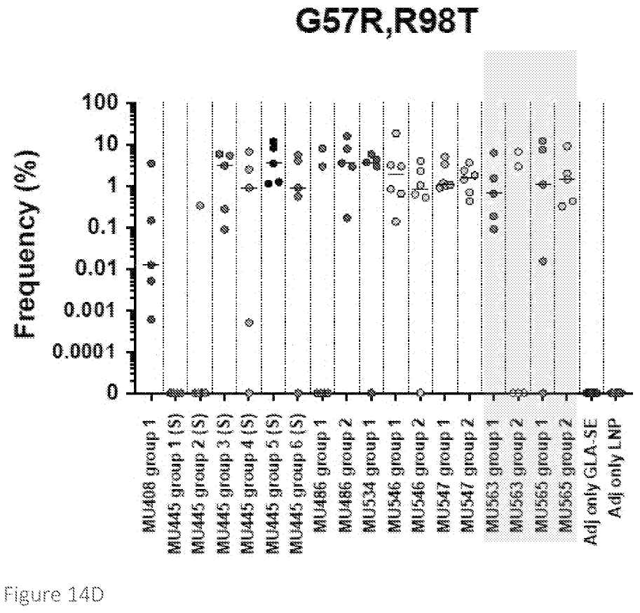

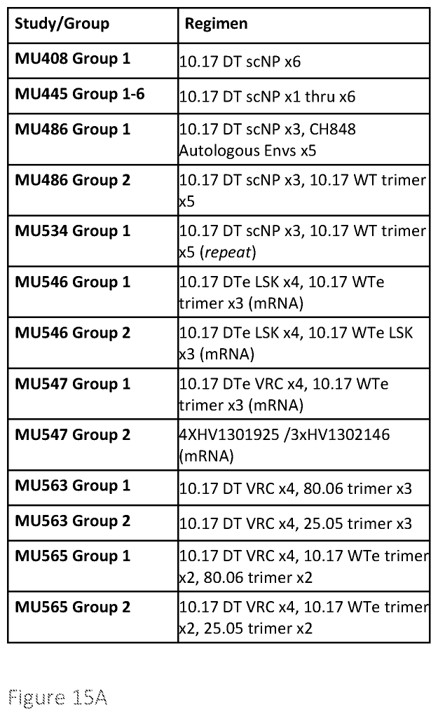

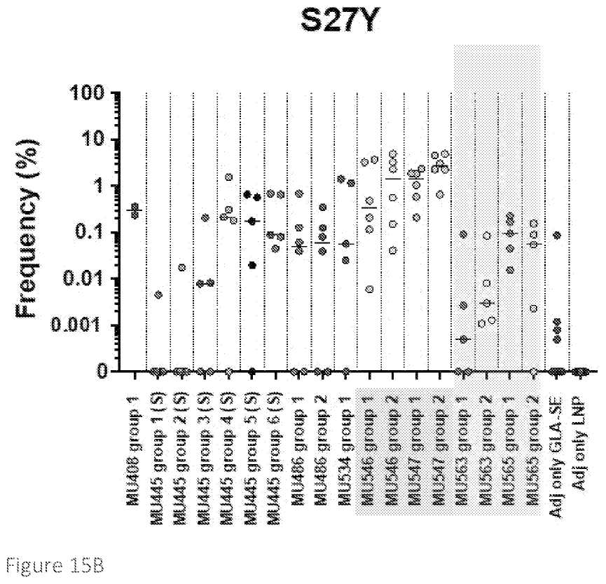

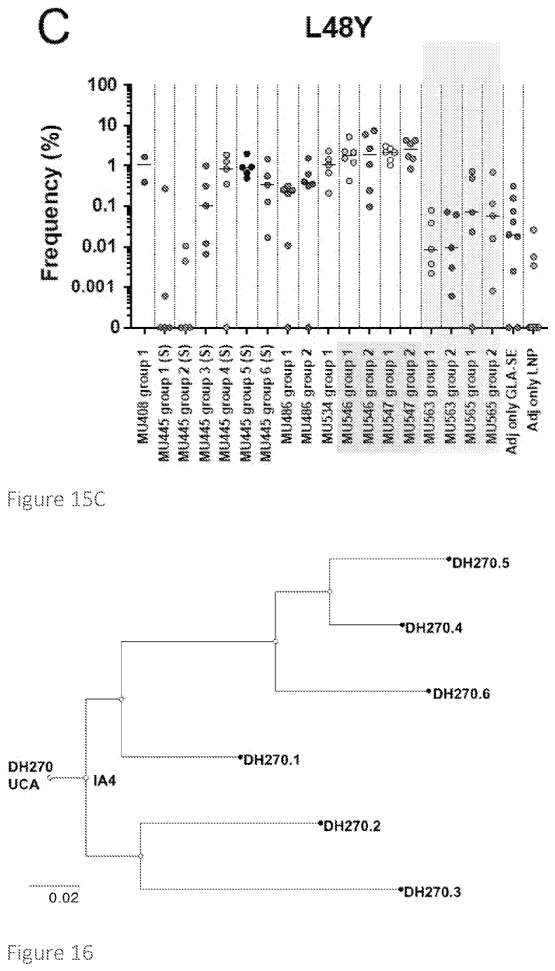

FIGS. 15A-C show Next generation sequencing of light chain variable regions shows vaccine selection of critical functional improbable mutations needed for DH270 antibody affinity maturation. In FIG. 15A, the table describes each DH270 UCA3 VH^{+/-}, VL^{+/-} mouse immunization regimen. FIGS. 15B,C, respectively, show the frequency of the observed somatic mutation. Adjuvant only groups are shown as Adj only GLA-SE for protein immunization and Adj only LNP for mRNA. Group median is shown by horizontal bars. Mu546 and Mu547 studies delivered the envelopes as mRNAs. All other groups used proteins. Mouse studies 563 and 565 included the D358.80.06 DS.ch.SOSIP and D526.25.05 DS.ch.SOSIP as boosting immunogens. Based on the frequency of somatic mutations observed in DH270 sequences, D358.80.06 DS.ch.SOSIP and D526.25.05 DS.ch.SOSIP did not select for higher frequencies of VH (G57R, R98T) mutations or VL (L48Y, S27Y) mutations than immunizing with CH848.D949.10.17N133D/N138T alone (Mu408 or Mu445 group 5). mRNA immunization selected for higher frequencies of VL (L48Y, S27Y) mutations than protein immunization with the same envelope.

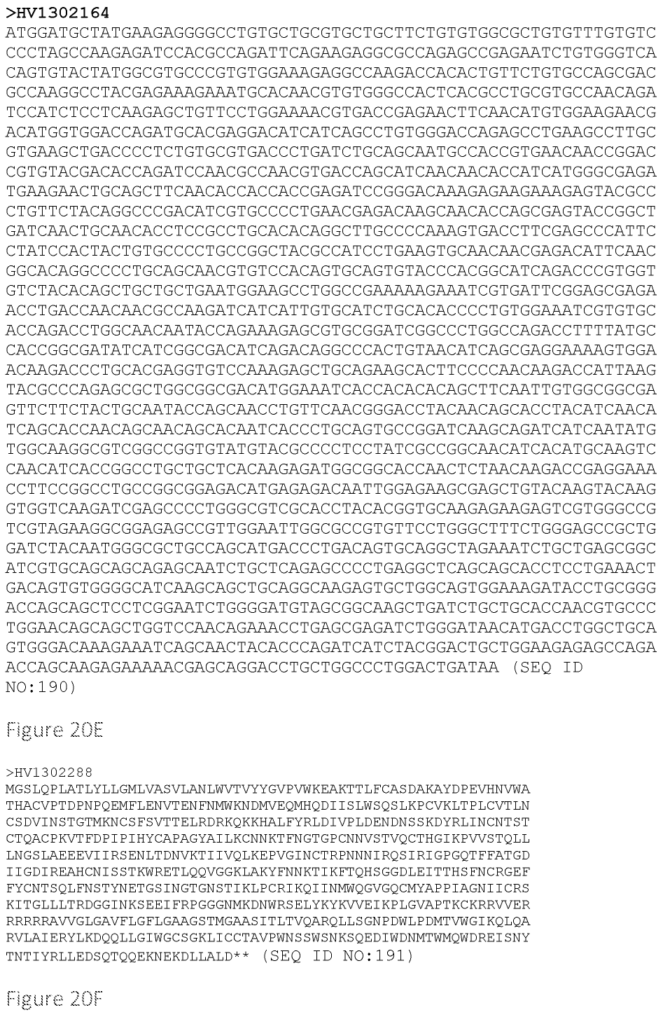

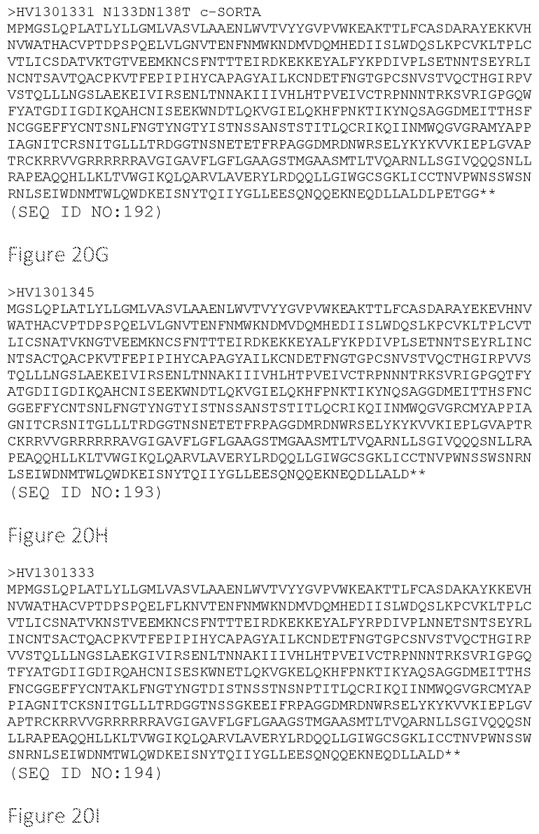

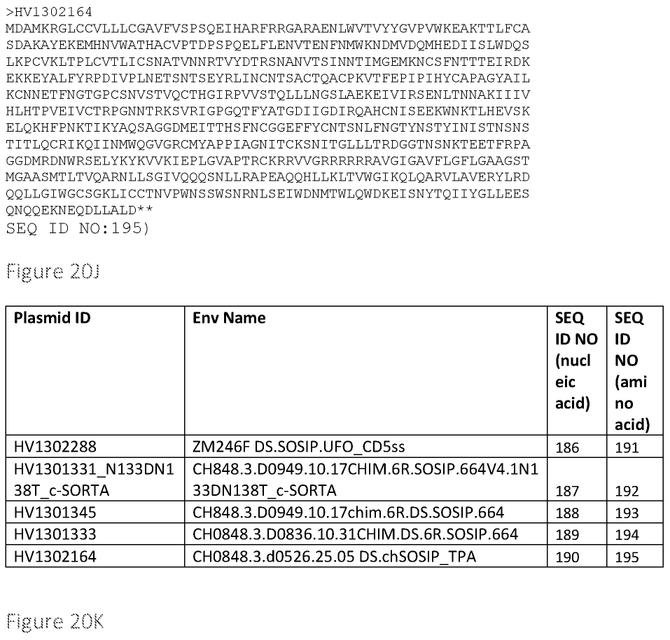

Non-limiting embodiments of envelopes used in mouse studies in Example 2 are shown in FIG. 20.

What is claimed is:

1. A recombinant HIV-1 envelope selected from the envelopes listed in Table 3, Table 4, FIGS. 1-3, or FIG. 20.

2. A composition comprising the envelope of claim 1 and a carrier, wherein the envelope is a protomer comprised in a trimer.

3. The composition of claim 2, wherein the envelope is comprised in a stable trimer.

4. A composition comprising a nanoparticle and a carrier, wherein the nanoparticle comprises any one of the envelopes of claim 1.

5. The composition of claim 4, wherein the nanoparticle is a ferritin self-assembling nanoparticle.

6. A composition comprising a nanoparticle and a carrier, wherein the nanoparticle comprises any one of the trimers of claim 2 or 3.

7. The composition of claim 6, wherein the nanoparticle is a ferritin self-assembling nanoparticle.

8. The composition of claim 7, wherein the nanoparticle comprises multimers of trimers.

9. The composition of claim 7, wherein the nanoparticle comprises one to eight trimers.

10. A method of inducing an immune response in a subject comprising administering an immunogenic composition comprising any one of the recombinant envelopes of the preceding claims or compositions of the preceding claims, in an amount sufficient to induce an immune response.

11. The method of claim 10, wherein the composition is administered as a prime.

12. The method of claim 10, wherein the composition is administered as a boost.

13. A nucleic acid encoding any of the recombinant envelopes of the preceding claims.

14. A composition comprising the nucleic acid of claim 13 and a carrier.

15. A method of inducing an immune response in a subject comprising administering an immunogenic composition comprising the nucleic acid of claim 13 or the composition of claim 14.

16. A method of inducing an immune response comprising administering an immunogenic composition comprising a prime immunogen from Table 4 followed by at least one boost immunogen from Table 4, wherein the boost immunogens are administered in the order appearing in Table 4, in an amount sufficient to induce an immune response.

17. The method of claim 16, further comprising administering a boost from Table 4, wherein the boost is CH848.0808.15.15 in any suitable form.

18. The method of claim 17, further comprising administering a boost from Table 4, wherein the boost is CH848.0358.80.06 in any suitable form.

19. The method of claim 17, further comprising administering a boost from Table 4, wherein the boost is CH848.1432.5.41 in any suitable form.

20. The method of claim 17, further comprising administering a boost from Table 4, wherein the boost is CH848.1621.4.44 in any suitable form.

21. The method of claim 17, further comprising administering a boost from Table 4, wherein the boost is CH848.1305.10.35 in any suitable form.

22. The method of claim 17, further comprising administering a boost from Table 4, wherein the boost is P0402.c2.11 (G) in any suitable form.

23. The method of claim 17, further comprising administering a boost from Table 4, wherein the boost is ZM246F (C) in any suitable form.

24. The method of claim 16-23, wherein the prime or boost immunogen are administered as a nanoparticle.

25. The method of claim 16-23, wherein the nanoparticle is a ferritin self-assembling nanoparticle.

26. The method of claim 16-23, wherein the prime or boost immunogen are administered as a mRNA-LNP formulation.
